(19) United States
(12) Patent Application Publication
(10) Pub. No.: US 2026/0297693 A1
(43) Pub. Date: Oct. 1, 2026

(54) DIGITAL DROPLET BASED ASSAY FOR DETECTING REPLICATION COMPETENT LENTIVIRAL VECTOR

(71) Applicant: Sana Biotechnology, Inc., Seattle, WA (US)

(72) Inventors: Andrew TUCKER, Seattle, WA (US); Aesha VAKIL, Seattle, WA (US); Semih TAREEN, Seattle, WA (US)

(73) Assignee: Sana Biotechnology, Inc., Seattle, WA (US)

(21) Appl. No.: 19/475,685

(22) PCT Filed: Apr. 18, 2024
(86) PCT No.: PCT/US2024/025079

§ 371 Date: Oct. 15, 2025

Related U.S. Application Data
(60) Provisional application No. 63/496,934, filed on Apr. 18, 2023.

(51) Int. Cl. C12Q 1/70; C12N 15/86; C12Q 1/686

(52) U.S. Cl. CPC C12Q 1/702; C12N 15/86; C12Q 1/686; C12N 2740/13043; C12N 2740/13051; C12N 2740/15043; C12N 2740/15051

(57) ABSTRACT

Among other things, the present disclosure provides methods for the detection of replication competent lentiviral vectors.

### CROSS-REFERENCE TO RELATED APPLICATIONS

The present application claims priority from U.S. Provisional Application No. 63/496,934 filed on Apr. 18, 2023 entitled “Digital Droplet Based Assay for Detecting Replication Competent Lentiviral Vector,” the contents of which are incorporated by reference in their entirety.

### BACKGROUND

Viral vectors provide an efficient means for modification of eukaryotic cells. Accordingly, the use of viral vectors continues to be important for both research and clinical gene therapy applications. One viral vector system that has been developed is the lentiviral vector system, which has been derived from the human immunodeficiency virus.

### SUMMARY

Lentivirus production using second or third generation self-inactivating and split genome plasmids minimize the risk of forming a replication competent lentivirus (RCL). While using second or third generation split genome plasmids should make the formation of RCL implausible, current U.S. Food and Drug Administration (FDA) guidelines include testing both the viral vector (e.g., used for gene or cell therapy) and producer cells to rule out RCL formation. Traditional RCL assays utilize the C8166 cell line and HIV-1 as a positive control with readouts based on p24 protein, quantitative PCR (qPCR), or product-enhanced reverse transciptase assay (PERT) to assess for the outgrowth of replication competent particles. Because of the low sensitivity of the end point readouts, these RCL assays can take 50-86 days and require significant amounts of vector material to complete. Furthermore, use of HIV-1 as a positive control introduces biosafety level and handling challenges. Although traditional C8166 cells are permissive to VSV-G pseudotyped lentiviral vectors that are commonly used for ex vivo cell therapy, these cells may not be permissive to novel vectors being developed for in vivo use with selective tropism, including retargeted viral fusogens pseudotyped on lentiviral vectors called “fusosomes.” The present disclosure provides, among other things, RCL assays that address the challenges experienced with traditional RCL assays. In particular, the present disclosure provides RCL assays that utilize a permissible suspension cell line for amplification with ddPCR endpoint using 4070A-MLV as a positive control. RCL assays described herein decrease the assay time from 56-86 days down to 9-18 days while maintaining a sensitive readout.

In one aspect the present disclosure provides a method of detecting replication competent lentivirus (RCL) comprising a fusogen, the method comprising:
(a) performing a nucleic acid amplification technique on a reaction mixture,
   wherein the reaction mixture comprises a test sample that comprises test nucleic acid,
   wherein the nucleic acid amplification technique is capable of amplifying a nucleic acid encoding at least a portion of the fusogen,
   wherein a fusogen amplicon will be produced if the nucleic acid encoding the fusogen of the RCL is present in the test nucleic acid; and
(b) determining whether the fusogen amplicon is produced by the nucleic acid amplification technique, wherein the presence of the fusogen amplicon indicates presence of RCL.

In some embodiments of the disclosed method, the nucleic acid technique is capable of amplifying a nucleic acid encoding a control sequence from a control virus, wherein a control amplicon will be produced if the nucleic acid encoding the control sequence is present in the test nucleic acid.

In some embodiments, the disclosed method further comprises determining whether a control amplicon is produced by ae nucleic acid amplification technique, wherein the presence of the control amplicon indicates the nucleic acid amplification technique was successfully performed.

In some embodiments of the disclosed method, a control virus is mouse leukemia virus (MLV) and the control sequence is a nucleic acid sequence encoding at least a portion of an MLV Env protein.

In some embodiments of the disclosed method, the nucleic acid amplification technique is single molecule PCR.

In some embodiments of the disclosed method, the nucleic acid amplification technique is ddPCR.

In some embodiments of the disclosed method, the test sample is obtained from a cell culture comprising cells permissible to lentiviral transduction.

In some embodiments of the disclosed method, the method further comprises obtaining the test sample from the cell culture.

In some embodiments of the disclosed method, the test sample comprises supernatant from the cell culture.

In some embodiments of the disclosed method, the cells permissible to lentiviral transduction have been transduced with a lentiviral vector.

In some embodiments of the disclosed method, the cells permissible to lentiviral transduction have been transduced with MLV.

In some embodiments of the disclosed method, the cells transduced with a lentiviral vector and the cells transduced with MLV are in different cultures.

In some embodiments of the disclosed method, the cells transduced with a lentiviral vector and the cells transduced with MLV are in the same cultures.

In some embodiments of the disclosed method, the transduction occurs in a 96 well plate, a T25 flask, a T75 flask, a T150 flask, or a T225 flask.

In some embodiments of the disclosed method, the cells in the culture are passaged at least once between transduction and collection of the supernatant.

In some embodiments of the disclosed method, the cells in the culture are passaged at least twice between transduction and collection of the supernatant.

In some embodiments of the disclosed method, the cells transduced with MLV are transduced at 1 infectious unit (IU), 10 IU, or 100 IU.

In some embodiments of the disclosed method, the cells permissible to lentiviral transduction are selected from B cells, T cells, natural killer cells, islet cells, glial progenitor cells, neuronal cells, hematopoietic stem cells, cardiac cells, hepatocytes, stem cells, or induced pluripotent stem cells.

In some embodiments of the disclosed method, the cells permissible to lentiviral transduction are T cells.

In some embodiments of the disclosed method, the cells permissible to lentiviral transduction are SupT1 cells.

In some embodiments of the disclosed method, the test sample is obtained at least 7, 8, 9, or 10 days after transduction with the lentiviral vector.

In some embodiments of the disclosed method, the test sample is obtained less than 56 days after transduction with the lentiviral vector.

In some embodiments of the disclosed method, the fusogen encoded by the nucleic acid is a viral fusogen.

In some embodiments of the disclosed method, the fusogen is involved in attachment of a viral vector to a cell membrane.

In some embodiments of the disclosed method, the fusogen is involved in directing fusion of the lipid bilayer of a viral vector and a cell membrane.

In some embodiments of the disclosed method, the fusogen comprises one or more paramyxovirus envelope proteins or portion thereof.

In some embodiments of the disclosed method, the one or more paramyxovirus envelope proteins or portion thereof comprises a paramyxovirus glycoprotein (“Protein G”) or a portion thereof.

In some embodiments of the disclosed method, the one or more paramyxovirus envelope proteins or portion thereof comprises a paramyxovirus fusion protein (“Protein F”) or portion thereof.

In some embodiments of the disclosed method, the fusogen has a tropism for the cells permissible to lentiviral transduction.

One aspect of the disclosure herein is a method of manufacturing a drug product, comprising:
performing the disclosed method of detecting RCL comprising a fusogen, wherein the test sample was obtained from a cell culture that had been transduced with a drug substance comprising a lentiviral vector.

In some embodiments of the disclosed method, the fusogen amplicon is not produced by the nucleic acid amplification technique.

In some embodiments of the disclosed method, the control amplicon is produced by the nucleic acid amplification technique.

In some embodiments of the disclosed method, the method further comprises adding a pharmaceutically acceptable excipient to the drug substance.

In some embodiments of the disclosed method of manufacturing, the test sample is collected prior to harvesting the cell culture.

In some embodiments of the disclosed method of manufacturing, further comprising treating the cell culture with a nuclease.

In some embodiments of the disclosed method of manufacturing, the test sample is collected before treating the cell culture with a nuclease.

In some embodiments of the disclosed method of manufacturing, the test sample is collected after treating the cell culture with a nuclease.

In some embodiments of the disclosed method of manufacturing, the presence of the fusogen amplicon above a predetermined threshold indicates presence of RCL.

Also provided herein is a method of manufacturing a drug product, comprising
(a) detecting replication competent lentivirus (RCL) in the drug product by:
   (i) performing a nucleic acid amplification technique on a reaction mixture,
      wherein the reaction mixture comprises a test sample that comprises test nucleic acid,
      wherein the nucleic acid amplification technique is capable of amplifying a nucleic acid encoding at least a portion of the fusogen,
      wherein a fusogen amplicon will be produced if the nucleic acid encoding the fusogen of the RCL is present in the test nucleic acid; and
   (ii) determining whether the fusogen amplicon is produced by the nucleic acid amplification technique, wherein the presence of the fusogen amplicon indicates presence of RCL.

### BRIEF DESCRIPTION OF THE DRAWING

The drawings are for illustration purposes only, not for limitation.

FIG. 1 shows an exemplary schematic detailing a traditional replication competent lentivirus (RCL) assay.

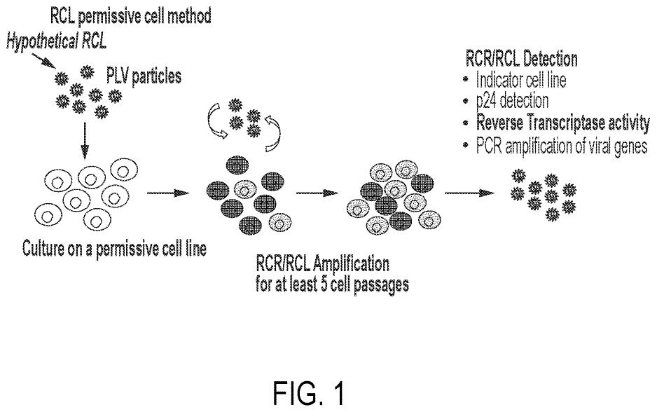

FIG. 2 shows exemplary criteria for assessing lentiviral production for uses in pharmaceutical applications. See, e.g., Farley, et al., 2015. Development of a replication-competent lentivirus assay for dendritic cell-targeting lentiviral vectors. Molecular Therapy-Methods & Clinical Development, 2, p. 15017, which is incorporated herein by reference in its entirety.

FIG. 3 shows exemplary G Amplicon, F Amplicon, and Mouse Leukemia Virus (MLV) Amplicon signals normalized to SupT1 normalization amplicon (e.g., hTert) and exemplary ddPCR amplitude cycles.

FIG. 4 shows exemplary experimental setup for larger-scale co-culture experiments.

FIG. 5 shows exemplary ddPCR signal for F Amplicon (here, NivF) in copies per UL and normalized to SupT1 normalization amplicon (e.g., uTert).

FIG. 6 shows ddPCR signal for G Amplicon (here, NivG) in copies per μL and normalized to SupT1 normalization amplicon (e.g., uTert).

FIG. 7 shows ddPCR signal for MLV Amplicon in copies per μL and normalized to SupT1 normalization amplicon (e.g., uTert).

### CERTAIN DEFINITIONS

In general, terminology used herein is in accordance with its understood meaning in the art, unless clearly indicated otherwise. Explicit definitions of certain terms are provided below; meanings of these and other terms in particular instances throughout this specification will be clear to those skilled in the art from context.

In order that the present invention may be more readily understood, certain terms are first defined below. Additional definitions for the following terms and other terms are set forth throughout the specification.

Administration: As used herein, the term “administration,” typically refers to application or delivery to a subject or system. Those of ordinary skill in the art, reading the present disclosure, will appreciate, for example, that a variety of routes are available for administration of compositions; for example, some compositions may be administered by one or more routes such as ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve dosing, application, or interaction that is intermittent (e.g., a plurality of doses separated in time) and/or periodic (e.g., individual doses separated by a common period of time) dosing. In some embodiments, administration may involve continuous dosing (e.g., perfusion), application or interaction for at least a selected period of time.

Crude Drug Substance: As used herein, the term “crude drug substance” is an intermediate composition generated in the production of a drug substance. In some embodiments, an active ingredient (e.g., viral vectors) in a crude drug substance have been enriched from other components (e.g., producer cell components, e.g., producer cell DNA and/or protein) that the active ingredient has been associated with during a production process. A crude drug substance often needs further processing to purify, isolate, or otherwise enrich the active ingredient prior to being classified as a drug substance or being incorporated into a drug product.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Drug Substance: As used herein, the term “drug substance” is an active ingredient (e.g., viral vectors) that is intended to furnish pharmacological activity or other direct effect in the diagnosis, cure, mitigation, treatment, or prevention of disease or to affect the structure or any function of a subject's body, but does not include intermediates used in the synthesis of such ingredient. A drug substance may need further processing to become a “drug product,” which is a finished dosage form (e.g., tablet or solution) to be administered to a subject. However, a drug substance does not require further processing to purify, isolate, or otherwise enrich the active ingredient prior to incorporation into a drug product.

Excipient: As used herein, refers to a non-therapeutic agent that may be included in a pharmaceutical composition, for example to provide or contribute to a desired consistency or stabilizing effect.

“Improved,” “increased,” “decreased” or “reduced”: As used herein, these terms, or grammatically comparable comparative terms, indicate values that are relative to a comparable reference measurement. For example, in some embodiments, an assessed value achieved with a method of interest may be “improved” relative to that obtained with a comparable reference method. Alternatively or additionally, in some embodiments, an assessed value achieved in a method of interest may be “improved” relative to that obtained in the same method under different conditions (e.g., prior to or after an event or step). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has a functional activity.

Plasmid: as used herein, refers to a nucleic acid molecule capable of transporting another nucleic acid to which it has been linked. Standard techniques may be used for recombinant DNA, oligonucleotide synthesis, and tissue culture and transformation (e.g., transfection, e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

Producer cell: As used herein, a “producer cell” is a cell capable of producing a viral vector when cultured under appropriate conditions. A number of cells are known to be capable of producing viral vectors, including for example, HEK293 cells, PER.C6 cells, VERO cells, 293T cells, A549 cells, MRC5 cells, HeLa cells, Sf9 cells, and BHK-21 cells.

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secreations, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc. In some embodiments, a sample may be a “crude” sample in that it has been subjected to relatively little processing and/or is complex in that it includes components of relatively varied chemical classes.

Subject: As used herein, the term “subject” refers an organism, typically a mammal (e.g., a human). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Tropism: As used herein, the term “tropism” refers to the ability of a molecule (e.g., a fusogen) or viral vector to interact with molecules (e.g., receptors or antigens) associated with a cell (e.g., in its cell membrane). For example, if a fusogen has a tropism for T cells, the fusogen is able to interact with molecules on the surface of or in the cell membrane of a T cell. In some embodiments, this interaction allows for the fusion of the viral vector with the membrane of the cell and ultimately entry into the cell.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls. The section headings used herein are for organizational purposes only and are not to be construed as limiting the subject matter described in any way.

### DETAILED DESCRIPTION OF CERTAIN EMBODIMENTS

### I. Lentiviral Vectors

In some embodiments, the present disclosure relates to testing of lentiviral vectors. In some embodiments, the present disclosure relates to detecting or determining if a lentiviral vector is replication competent. One of skill in the art will be aware of several methods to generate a lentiviral vector.

In some embodiments, one or more nucleic acids comprise plasmids for the production of a viral vector, e.g., lentiviral vector. Typically, a lentiviral vector system will comprise a packaging plasmid, an envelope plasmid, and a transfer plasmid encoding a transgene. Typically, a lentiviral vector is produced when a producer cell receives one or more plasmids of a lentiviral vector system. One of skill in the art will appreciate that multiple generations of lentiviral vector systems exist that vary in number of plasmids utilized and the protein(s) each plasmid encodes. The present disclosure appreciates that one or more lentiviral vector(s) produced using one or more approach(es) (e.g., first, second, third, or fourth generation lentiviral vector systems) may be tested by the methods described herein. As one skilled in the art would appreciate, the present disclosure is not bound to the testing of lentiviral vectors and/or related nucleic acids produced by any one particular method.

### A. First Generation

A first-generation lentiviral vector system typically includes three plasmids: a packaging plasmid, an envelope plasmid, and a transfer plasmid. A packaging plasmid of a first-generation lentivirus system usually encodes one or more accessory polypeptides (such as viral infectivity factor (Vif), viral protein r (Vpr), viral protein u (Vpu), and negative factor (Nef)) and one or more polypeptides involved in viral production (such as group-specific antigen (Gag), polymerase (Pol), regulator of virion (Rev), and trans-activator of transcription (Tat) genes).

An envelope plasmid of a first-generation lentiviral vector system typically encodes an envelope protein (Env) which is usually the HIV-1 Env glycoprotein or the VSV-G glycoprotein.

A first-generation lentiviral vector system can also include a transfer plasmid which typically has a 5′ long terminal repeat (LTR), a Rev responsive element, a sequence for a promoter of interest, a sequence encoding a transgene of interest, and a 3′ LTR. The LTRs typically comprise a U3, an R, and a U5 region. A transgene as described herein can encode any gene product (e.g., RNA or polypeptide). Exemplary transgenes and encoded gene products are described herein.

In some embodiments, a lentiviral vector system disclosed herein is a first-generation lentiviral vector system. In some embodiments, a lentivirus system comprises one or more plasmids of a first-generation lentiviral vector system as disclosed herein. In some embodiments, a lentivirus system comprises one or more packaging plasmids, one or more envelope plasmids, and one or more transfer plasmids of a first-generation lentiviral vector system. In some embodiments, a lentivirus system comprises a packaging plasmid, an envelope plasmid and a transfer plasmid of a first-generation lentiviral vector system. In some embodiments, a lentivirus system comprises a packaging plasmid, two envelope plasmids and a transfer plasmid of a first-generation lentiviral vector system.

In some embodiments, when a lentiviral vector system disclosed herein is a first-generation lentiviral vector system, one or more envelope plasmids do not encode an Env polypeptide. In some embodiments, one or more envelope plasmids of a lentiviral vector system encode one or more fusogens or a biologically active portions thereof. In some embodiments, a fusogen comprises a glycoprotein, e.g., G protein or portion thereof, and/or F protein or portions thereof. In some embodiments, one or more envelope plasmids encode paramyxovirus glycoprotein G (G protein) or a portion thereof and paramyxovirus fusion protein (F protein) or a portion thereof. In some embodiments, a fusogen comprises a chimeric protein. Various fusogens are contemplated as discussed further herein.

### B. Second Generation

A second-generation lentiviral vector system also generally includes three plasmids, similar to a first-generation lentiviral vector system. The three plasmids of a second-generation lentiviral vector system are: a packaging plasmid, an envelope plasmid and a transfer plasmid. One of the differences in a second-generation lentiviral vector system as compared to the earlier generation is that the packaging plasmid does not encode viral accessory polypeptides Vif, Vpr, Vpu, and Nef. In a second-generation lentiviral vector system, the packaging plasmid only encodes the Gag, Pol, Tat and Rev polypeptides.

In some embodiments, a lentiviral vector system disclosed herein is a second-generation lentiviral vector system as disclosed herein. In some embodiments, a lentivirus system comprises one or more plasmids of a second-generation lentiviral vector system as disclosed herein. In some embodiments, a lentivirus system comprises one or more packaging plasmids, one or more envelope plasmids, and one or more transfer plasmids of a second-generation lentiviral vector system. In some embodiments, a lentivirus system comprises a packaging plasmid, an envelope plasmid and a transfer plasmid of a second-generation lentiviral vector system. In some embodiments, a lentivirus system comprises a packaging plasmid, two envelope plasmids and a transfer plasmid of a second-generation lentiviral vector system.

In some embodiments, when a lentiviral vector system disclosed herein is a second-generation lentiviral vector system, one or more envelope plasmids do not encode an Env polypeptide. In some embodiments, one or more envelope plasmids of a lentiviral vector system encode one or more fusogens or a biologically active portions thereof. In some embodiments, a fusogen comprises a glycoprotein, e.g., G protein or portion thereof, and/or F protein or portions thereof. In some embodiments, one or more envelope plasmids encode paramyxovirus glycoprotein G (G protein) or a portion thereof and paramyxovirus fusion protein (F protein) or a portion thereof. In some embodiments, a fusogen comprises a chimeric protein. Various fusogens are contemplated as discussed further herein.

### C. Third Generation

Third-generation lentiviral vector systems are newer and were developed to increase the safety of earlier generation vector systems. In the third-generation lentiviral vector system there are generally four plasmids: a packaging plasmid, an envelope plasmid, a regulatory plasmid, and a transfer plasmid. The envelope plasmid of third-generation lentiviral vector systems is relatively unchanged from envelope plasmids in prior generations in that it encodes an Env polypeptide. The packaging and transfer plasmids have several differences as described herein.

In a third-generation lentiviral vector system, the packaging plasmid only encodes the Gag and Pol polypeptides. A separate plasmid, the regulatory plasmid encodes the Rev polypeptide. The transfer plasmid also includes changes particularly to the LTRs. To enhance safety, the LTRs were modified in the U3 region. A transfer plasmid of a third-generation lentiviral vector system includes LTR regions comprising an R element, a U5 element, an RRE element, a posttranscriptional regulatory elements (PREs), and a self-inactivating (SIN) region.

In some embodiments, a lentiviral vector system disclosed herein is a third-generation lentiviral vector system as disclosed herein. In some embodiments, a lentivirus system comprises one or more plasmids of a third-generation lentiviral vector system as disclosed herein. In some embodiments, a lentivirus system comprises one or more packaging plasmids, one or more envelope plasmids, one or more regulatory plasmids, and one or more transfer plasmids of a third-generation lentiviral vector system. In some embodiments, a lentivirus system comprises a packaging plasmid, a regulatory plasmid, an envelope plasmid and a transfer plasmid of a third-generation lentiviral vector system. In some embodiments, a lentivirus system comprises a packaging plasmid, a regulatory plasmid, two envelope plasmids and a transfer plasmid of a third-generation lentiviral vector system.

In some embodiments, when a lentiviral vector system disclosed herein is a third-generation lentiviral vector system, one or more envelope plasmids do not encode an Env polypeptide. In some embodiments, one or more envelope plasmids of a lentiviral vector system encode one or more fusogens or a biologically active portions thereof. In some embodiments, a fusogen comprises a glycoprotein, e.g., G protein or portion thereof, and/or F protein or portions thereof. In some embodiments, one or more envelope plasmids encode paramyxovirus glycoprotein G (G protein) or a portion thereof and paramyxovirus fusion protein (F protein) or a portion thereof. In some embodiments, a fusogen comprises a chimeric protein. Various fusogens are contemplated as discussed further herein.

### D. Fourth Generation

Further iterations of lentiviral vector systems have been recently developed. For example, a fourth-generation lentiviral vector system having more than four plasmids has been reported to increases the number of recombination events required to generate replication-competent lentivirus (RCL). Such a fourth-generation lentiviral vector typically includes one or more plasmids whose expression is driven by a Tet-Off and/or Tat transactivator. A fourth-generation lentiviral vector system can include five plasmids, wherein one or more plasmid(s) is/are pTre-gag-pro, LTRHIV-vpr-pol, pCMV-VSVG, pMV-tet-off, and pTre-tat-ires-rev.

In some embodiments, a lentiviral vector system disclosed herein is a fourth-generation lentiviral vector system as disclosed herein. In some embodiments, a lentivirus system comprises one or more plasmids of a fourth-generation lentiviral vector system as disclosed herein.

In some embodiments, when a lentiviral vector system disclosed herein is a fourth-generation lentiviral vector system, one or more envelope plasmids do not encode an Env polypeptide. In some embodiments, one or more envelope plasmids of a lentiviral vector system encode one or more fusogens or a biologically active portions thereof. In some embodiments, a fusogen comprises a glycoprotein, e.g., G protein or portion thereof, and/or F protein or portions thereof. In some embodiments, one or more envelope plasmids encode paramyxovirus glycoprotein G (G protein) or a portion thereof and paramyxovirus fusion protein (F protein) or a portion thereof. In some embodiments, a fusogen comprises a chimeric protein. Various fusogens are contemplated as discussed further herein.

### E. Certain Exemplary Plasmids

In some embodiments, a lentiviral vector system disclosed herein is a first-generation lentiviral vector system as disclosed herein. In some embodiments, a lentivirus system comprises a packaging plasmid, two envelope plasmids, and a transfer plasmid of a first-generation lentiviral vector system. In some embodiments, the envelope plasmids do not encode an Env polypeptide. In some embodiments, the envelope plasmids encode one or more fusogens or a biologically active portions thereof. In some embodiments, the one or more fusogens comprise one or more glycoproteins, e.g., G protein or a portion thereof, and/or F protein or a portion thereof. In some embodiments, a first envelope plasmid encodes a G protein or a biologically active portion thereof. In some embodiments, a second envelope plasmid encodes a F protein or a biologically active portion thereof. In some embodiments of such a lentiviral vector system, a transfer plasmid comprise a sequence encoding a transgene disclosed herein. In some embodiments, a transgene is or comprises a Chimeric Antigen Receptor, e.g., as disclosed herein.

In some embodiments, a lentiviral vector system disclosed herein is a second-generation lentiviral vector system as disclosed herein. In some embodiments, a lentivirus system comprises a packaging plasmid, two envelope plasmids, and a transfer plasmid of a second-generation lentiviral vector system. In some embodiments, the envelope plasmids do not encode an Env polypeptide. In some embodiments, the envelope plasmids encode one or more fusogens or a biologically active portions thereof. In some embodiments, the one or more fusogens comprise one or more glycoproteins, e.g., G protein or a portion thereof, and/or F protein or a portion thereof. In some embodiments, a first envelope plasmid encodes a G protein or a biologically active portion thereof. In some embodiments, a second envelope plasmid encodes a F protein or a biologically active portion thereof. In some embodiments of such a lentiviral vector system, a transfer plasmid comprise a sequence encoding a transgene disclosed herein. In some embodiments, a transgene is or comprises a Chimeric Antigen Receptor, e.g., as disclosed herein.

In some embodiments, a lentiviral vector system disclosed herein is a third-generation lentiviral vector system as disclosed herein. In some embodiments, a lentivirus system comprises a packaging plasmid, a regulatory plasmids, two envelope plasmids, and a transfer plasmid of a third-generation lentiviral vector system. In some embodiments, the envelope plasmids do not encode an Env polypeptide. In some embodiments, the envelope plasmids encode one or more fusogens or a biologically active portions thereof. In some embodiments, the one or more fusogens comprise one or more glycoproteins, e.g., G protein or a portion thereof, and/or F protein or a portion thereof. In some embodiments, a first envelope plasmid encodes a G protein or a biologically active portion thereof. In some embodiments, a second envelope plasmid encodes a F protein or a biologically active portion thereof. In some embodiments of such a lentiviral vector system, a transfer plasmid comprise a sequence encoding a transgene disclosed herein. In some embodiments, a transgene is or comprises a Chimeric Antigen Receptor, e.g., as disclosed herein.

In some embodiments, the lentiviral vector particle, comprises one or more of gag polyprotein, polymerase (e.g., pol), integrase (e.g., a functional or non-functional variant), protease, and a fusogen. In some embodiments, the viral particle further comprises rev. In some embodiments, one or more of the aforesaid proteins are encoded in the lentiviral genome (i.e., the insert as described above), and in some embodiments, one or more of the aforesaid proteins are provided in trans, e.g., by a helper cell, helper virus, or helper plasmid. In some embodiments, the lentiviral nucleic acid comprises one or more of the following nucleic acid sequences: 5′ LTR (e.g., comprising U5 and lacking a functional U3 domain), Psi packaging element (Psi, Y′), Central polypurine tract (cPPT) Promoter operatively linked to the payload gene, payload gene (optionally comprising an intron before the open reading frame), Poly A tail sequence, WPRE, and 3′ LTR (e.g., comprising U5 and lacking a functional U3). In some embodiments, the lentiviral nucleic acid further comprisesa cis-acting RNA packaging element, and a cPPT/CTS element. In some embodiments the lentiviral nucleic acid further comprises one or more insulator elements.

In some embodiments, the lentiviral vector comprises supramolecular complexes formed by viral proteins that self-assemble into capsids (e.g., viral capsids or viral nucleocapsids).

In some embodiments, the lentivirus packages nucleic acids from host cells carrying one or more viral nucleic acids (e.g., lentiviral nucleic acids) during the expression process. In some embodiments, the nucleic acids do not encode any genes involved in virus replication.

In some embodiments, the lentiviral nucleic acid comprises one or more of (e.g., all of): a 5′ promoter (e.g., to control expression of the entire packaged RNA), a 5′ LTR (e.g., that includes R (polyadenylation tail signal) and/or U5 which includes a primer activation signal), a primer binding site, a psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and U5). In some embodiments, the lentiviral nucleic acid further comprises one or more of a cPPT, a WPRE, and/or an insulator element.

A lentivirus typically replicates by reverse transcription of its genomic RNA into a linear double-stranded DNA copy and subsequently covalently integrates its genomic DNA into a host genome.

Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) are used.

A lentiviral vector can comprise a nucleic acid molecule (e.g., a transfer plasmid) that includes virus-derived nucleic acid elements that typically facilitate transfer of a nucleic acid molecule (e.g. including nucleic acid encoding an exogenous agent) or integration into the genome of a cell or to a viral particle that mediates nucleic acid transfer. Lentiviral vector particles will typically include various viral components and sometimes also host cell components in addition to nucleic acid(s). A lentiviral vector can comprise a virus or viral particle capable of transferring a nucleic acid into a cell (e.g. nucleic acid encoding an exogenous agent), or to the transferred nucleic acid (e.g., as naked DNA). Lentiviral vectors and transfer plasmids can comprise structural and/or functional genetic elements that are primarily derived from a virus. A lentiviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some vectors described herein, at least part of one or more protein coding regions that contribute to or are essential for replication may be absent compared to the corresponding wild-type virus. This makes the viral vector replication-defective. In some embodiments, the vector is capable of transducing a target non-dividing host cell and/or integrating its genome into a host genome.

The structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and

RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). The LTRs are involved in proviral integration and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are typically similar (e.g., identical) sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For the lentiviral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. Some lentiviruses comprise any one or more of the following genes that code for proteins that are involved in the regulation of gene expression: tot, rev, tax and rex. With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction promotes infection, e.g., by fusion of the viral membrane with the cell membrane.

In a replication-defective lentiviral vector genome gag, pol and env may be absent or not functional. The R regions at both ends of the RNA are typically repeated sequences. U5 and U3 represent unique sequences at the 5′ and 3′ ends of the RNA genome respectively.

Lentiviruses may also contain additional genes which code for proteins other than gag, pol and env. Examples of additional genes include (in HIV), one or more of vif, vpr, vpx, vpu, tat, rev and nef. EIAV has (amongst others) the additional gene S2. Proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11). The mechanisms of action of these two proteins are thought to be broadly similar to the analogous mechanisms in the primate viruses. In addition, an EIAV protein, Ttm, has been identified that is encoded by the first exon of tat spliced to the env coding sequence at the start of the transmembrane protein.

In addition to protease, reverse transcriptase and integrase, non-primate lentiviruses contain a fourth pol gene product which codes for a dUTPase. This may play a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. Infection of the target cell can comprise reverse transcription and integration into the target cell genome. The RLV typically carries non-viral coding sequences which are to be delivered by the vector to the target cell, such as nucleic acid encoding an exogenous agent as described herein. In embodiments, an RLV is incapable of independent replication to produce infectious retroviral particles within the target cell. Usually the RLV lacks a functional gag-pol and/or env gene and/or other genes involved in replication. The vector may be configured as a split-intron vector, e.g., as described in PCT patent application WO 99/15683, which is herein incorporated by reference in its entirety.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

A minimal lentiviral genome may comprise, e.g., (5′) R-U5-one or more first nucleotide sequences-U3-R (3′). However, the plasmid vector used to produce the lentiviral genome within a source cell can also include transcriptional regulatory control sequences operably linked to the lentiviral genome to direct transcription of the genome in a source cell. These regulatory sequences may comprise the natural sequences associated with the transcribed retroviral sequence, e.g., the 5′ U3 region, or they may comprise a heterologous promoter such as another viral promoter, for example the CMV promoter. Some lentiviral genomes comprise additional sequences to promote efficient virus production. For example, in the case of HIV, rev and RRE sequences may be included. Alternatively or in combination, codon optimization may be used, e.g., the gene encoding the exogenous agent may be codon optimized, e.g., as described in WO 01/79518, which is herein incorporated by reference in its entirety. Alternative sequences which perform a similar or the same function as the rev/RRE system may also be used. For example, a functional analogue of the rev/RRE system is found in the Mason Pfizer monkey virus. This is known as CTE and comprises an RRE-type sequence in the genome which is believed to interact with a factor in the infected cell. The cellular factor can be thought of as a rev analogue. Thus, CTE may be used as an alternative to the rev/RRE system. In addition, the Rex protein of HTLV-I can functionally replace the Rev protein of HIV-I. Rev and Rex have similar effects to IRE-BP.

In some embodiments, a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non dividing cells.

The deletion of additional genes may permit vectors to be produced without the genes associated with disease in lentiviral (e.g. HIV) infections. In particular, tat is associated with disease. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO 99/32646 and in WO 98/17815.

In some embodiments, the lentiviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. Thus, the concentration of free dNTPs in the cytoplasm increases as Vpx degrades SAMHD1 and reverse transcription activity is increased, thus facilitating reverse transcription of the retroviral genome and integration into the target cell genome.

Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. An additional description of codon optimization is found, e.g., in WO 99/41397, which is herein incorporated by reference in its entirety.

Many viruses, including HIV and other lentiviruses, use a large number of rare codons and by changing these to correspond to commonly used mammalian codons, increased expression of the packaging components in mammalian producer cells can be achieved.

In some embodiments, codon optimization has a number of other advantages. In some embodiments, by virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

In some embodiments, derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

In some embodiments, due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

It is within the level of a skilled artisan to empirically determine appropriate codon optimization of viral sequences. The strategy for codon optimized sequences, including gag-pol sequences, can be used in relation to any retrovirus, e.g., EIAV, FIV, BIV, CAEV, VMR, SIV, HIV-I and HIV-2. In addition this method can be used to increase expression of genes from HTLV-I, HTLV-2, HFV, HSRV and human endogenous retroviruses (HERV), MLV and other retroviruses.

In embodiments, the lentiviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

In some embodiments, the lentiviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the lentiviral proteins are derived from the same lentivirus. In some embodiments the lentiviral proteins are derived from more than one lentivirus, e.g. 2, 3, 4, or more lentiviruses.

In some embodiments, the gag and pol coding sequences are generally organized as the Gag-Pol Precursor in native lentivirus. The gag sequence codes for a 55-kD Gag precursor protein, also called p55. The p55 is cleaved by the virally encoded protease (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

In some embodiments, the lentiviral vector is integration-deficient. In some embodiments, the pol is integrase deficient, such as by encoding due to mutations in the integrase gene. For example, the pol coding sequence can contain an inactivating mutation in the integrase, such as by mutation of one or more of amino acids involved in catalytic activity, i.e. mutation of one or more of aspartic 64, aspartic acid 116 and/or glutamic acid 152. In some embodiments, the integrase mutation is a D64V mutation. In some embodiments, the mutation in the integrase allows for packaging of viral RNA into a lentivirus. In some embodiments, the mutation in the integrase allows for packaging of viral proteins into a lentivirus. In some embodiments, the mutation in the integrase reduces the possibility of insertional mutagenesis. In some embodiments, the mutation in the integrase decreases the possibility of generating replication-competent recombinants (RCRs) (Wanisch et al. 2009. Mol Ther. 1798): 1316-1332). In some embodiments, native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In some embodiments, the lentiviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

According to certain specific embodiments, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. A variety of lentiviral vectors are described in Naldini et ah, (1996a, 1996b, and 1998); Zufferey et al., (1997); Dull et al., 1998, U.S. Pat. Nos. 6,013,516; and 5,994,136, many of which may be adapted to produce a retroviral nucleic acid.

At each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

In some embodiments, a packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi [I] sequence) for encapsidation of the viral genome.

In various embodiments, lentiviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. In some aspects, provided herein is a replication incompetent (also referred to herein as replication defective) vector particle, that cannot participate in replication in the absence of the packaging cell (i.e., viral vector particles are not produced from the transduced cell). In some aspects, this is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly(A) sequence The 3′ LTR, the 5′ LTR, or both 3′ and 5′ LTRs, may be modified LTRs. Other modifications to the viral vector, i.e., retroviral or lentiviral vector, to render said vector replication incompetent are known in the art.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

The R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

The retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et ah, 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-L

In embodiments, a lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65:1053; and Cullen et al., 1991. Cell 58:423), and the hepatitis B virus post-transcriptional regulatory element (HPRE), which are herein incorporated by reference in their entireties. Generally, the RNA export element is placed within the 3′ UTR of a gene, and can be inserted as one or multiple copies.

In some embodiments, expression of heterologous sequences (e.g. nucleic acid encoding an exogenous agent) in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE

In some embodiments, a lentiviral nucleic acid described herein lacks or does not comprise a posttranscriptional regulatory element such as a WPRE or HPRE.

Elements directing the termination and polyadenylation of the heterologous nucleic acid transcripts may be included, e.g., to increases expression of the exogenous agent.

Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATT AAA, AGTAAA, a bovine growth hormone poly A sequence (BGHpA), a rabbit b-globin polyA sequence (rPgpA), or another suitable heterologous or endogenous polyA sequence.

In some embodiments, a lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Y′) packaging signal, RRE), and/or other elements that increase exogenous gene expression (e.g., poly (A) sequences), and may optionally comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

### II. Fusogens

In some embodiments, one or more viral vectors, e.g., lentiviral vectors, comprise one or more fusogens. In some embodiments, one or more fusogens comprise at least one fusogen that is involved in attachment of a viral vector to a cell membrane. In some embodiments, one or more fusogens comprise at least one fusogen that is involved in directing fusion of the lipid bilayer of a viral vector to a cell membrane. In some embodiments, one or more fusogens comprise one or more paramyxovirus envelope proteins or portion thereof. In some embodiments, the paramyxovirus envelope attachment proteins and/or retargeted attachment proteins provided herein exhibit fusogenic activity to a target cell, such as to deliver an exogenous agent or nucleic acid exogenous agent to the target cell.

In some embodiments, the paramyxovirus attachment protein is or comprises a hemagglutinin-neuraminidase (HN) from a respiratory paramyxovirus. In some embodiments, the respiratory paramyxovirus is a Sendai virus. The HN glycoproteins of Sendai viruses function to attach to sialic acids via the HN protein, and to mediate cell fusion for entry to cells via the F (fusion) protein. In some embodiments, the paramyxovirus attachment protein is or comprises a HN protein from the murine parainfluenza virus type 1 (See e.g., U.S. Pat. No. 10,704,061).

In some embodiments, the paramyxovirus attachment protein is or comprises a Nipah virus protein G, a measles protein H, a tupaia paramyxovirus H protein, a paramyxovirus G protein, a paramyxovirus H protein, a paramyxovirus HN protein, a Morbilivirus H protein, a respirovirus HN protein, a sendai HN protein, a rubulavirus HN protein, an avulavirus HN protein, or a derivative thereof. In some embodiments, the paramyxovirus attachment protein is or comprises a sequence chosen from Nipah virus G proteins, measles virus H proteins, tupaia paramyxovirus H proteins, paramyxovirus G proteins and H proteins and HN proteins, Hendra virus G proteins, Henipavirus G proteins, Morbilivirus H proteins, respirovirus HN protein, a Sendai virus HN protein, rubulavirus HN proteins, or avulavirus HN proteins, or a derivative thereof, or any combination thereof.

### A. Paramyxovirus Attachment Proteins

In some embodiments, the viral particles provided herein comprise a paramyxovirus envelope attachment protein, a first paramyxovirus envelope attachment protein, and/or a second paramyxovirus envelope attachment protein. In some embodiments, the paramyxovirus envelope attachment protein may be an envelope glycoprotein G, H and/or HN of the Paramyxoviridae family.

In some embodiments, the viral particles provided herein comprise a first paramyxovirus envelope attachment protein, a second paramyxovirus envelope attachment protein, and a third paramyxovirus envelope attachment protein. In some embodiments, each of the first, second, and third paramyxovirus envelope attachment protein may independently be an envelope glycoprotein G, H and/or HN of the Paramyxoviridae family.

In some embodiments, the viral particles provided herein comprise a first paramyxovirus envelope attachment protein, a second paramyxovirus envelope attachment protein, a third paramyxovirus envelope attachment protein, and one or more additional paramyxovirus envelope attachment proteins, such as a fourth paramyxovirus envelope attachment protein, or a fourth and fifth paramyxovirus envelope attachment protein, or a fourth, fifth, and sixth paramyxovirus envelope attachment protein, or beyond. In some embodiments, each of the paramyxovirus envelope attachment proteins may independently be an envelope glycoprotein G, H and/or HN of the Paramyxoviridae family.

In some embodiments, the paramyxovirus envelope attachment protein, first paramyxovirus envelope attachment protein, and/or second paramyxovirus envelope attachment protein and/or the third paramyxovirus envelope attachment protein and/or the fourth paramyxovirus envelope attachment protein and/or the fifth paramyxovirus envelope attachment protein and/or the sixth paramyxovirus envelope attachment protein, and/or any additional paramyxovirus envelope attachment protein is an attachment glycoprotein G (G protein) or biologically active portion thereof.

In some embodiments, the viral particle comprises a retargeted attachment protein, a first retargeted attachment protein, and/or second retargeted attachment protein exposed on the surface of the targeted viral particle. In some embodiments, the viral particle further comprises a third retargeted attachment protein exposed on the surface of the targeted viral particle. In some embodiments, the viral particle further comprises a third retargeted attachment protein and a fourth retargeted attachment protein exposed on the surface of the targeted viral particle. In some embodiments, the viral particle further comprises a third retargeted attachment protein, a fourth retargeted attachment protein, and a fifth retargeted attachment protein exposed on the surface of the targeted viral particle. In some embodiments, the viral particle further comprises a third retargeted attachment protein, a fourth retargeted attachment protein, a fifth retargeted attachment protein, and one or more additional retargeted attachment proteins, exposed on the surface of the targeted viral particle. In some embodiments, the retargeted attachment protein is or comprises a paramyxovirus attachment protein, wherein the paramyxovirus attachment protein is an attachment glycoprotein G (G protein) or biologically active portion thereof. In some embodiments, the retargeted attachment protein is or comprises a paramyxovirus attachment protein, wherein the paramyxovirus attachment protein is an attachment glycoprotein G (G protein) or biologically active portion thereof, and comprises a targeting moiety directed to a target molecule, e.g., a binding domain or a binding agent, expressed on the surface of a target cell.

The envelope attachment G proteins are type II transmembrane glycoproteins containing an N-terminal cytoplasmic tail (e.g. corresponding to amino acids 1-49 of SEQ ID NO: 3), a transmembrane domain (e.g. corresponding to amino acids 50-70 of SEQ ID NO:3), and an extracellular domain containing an extracellular stalk (e.g. corresponding to amino acids 71-187 of SEQ ID NO:3), and a globular head (corresponding to amino acids 188-602 of SEQ ID NO: 3). The N-terminal cytoplasmic domain is within the inner lumen of the lipid bilayer and the C-terminal portion is the extracellular domain that is exposed on the outside of the lipid bilayer. Regions of the stalk in the C-terminal region (e.g., corresponding to amino acids 71-187 of SEQ ID NO: 3) have been shown to be involved in interactions with F protein and triggering of F protein fusion (Liu et al. 2015 J of Virology 89:1838). In wild-type G protein, the globular head mediates receptor binding to henipavirus entry receptors Ephrin B2 and Ephrin B3, but is dispensable for membrane fusion (Brandel-Tretheway et al. Journal of Virology. 2019. 93(13)e00577-19).

In some embodiments herein, tropism of the G protein is altered by linkage of the G protein or biologically active fragment thereof (e.g., cytoplasmic truncation) to a sdAb variable domain. Binding of the G protein to a binding partner can trigger fusion mediated by a compatible paramyxovirus fusion protein (e.g., F protein) or biologically active portion thereof (such as any of the F proteins described in II.B below). G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved between henipavirus species. For example, the G protein of NiV and HeV viruses share 79% amino acids identity. Studies have shown a high degree of compatibility among G proteins with F proteins of different species as demonstrated by heterotypic fusion activation (Brandel-Tretheway et al. Journal of Virology. 2019). As described, a viral particle can contain at least two envelope attachment proteins (e.g., co-fusogens). In particular embodiments, the F protein or the functionally active variant or biologically active portion thereof retains fusogenic activity in conjunction with the at least two envelope attachment proteins (e.g., co-fusogens that are paramyxovirus attachment protein Gs) as provided, such as any set forth below. Fusogenic activity includes the activity of the paramyxovirus fusion protein (e.g., F protein) in conjunction with a G protein to promote or facilitate fusion of two membrane lumens, such as the lumen of the viral particle provided herein (e.g. having embedded in its lipid bilayer, such as exposed on its surface, at least two G proteins and a F protein), and a cytoplasm of a target cell, e.g. a cell that contains a surface receptor or molecule that is recognized or bound by the G protein.

Exemplary Henipavirus protein G sequences are provided in Table 1

Table 1. Henipavirus protein G sequence clusters. Column 1, Genbank ID includes the Genbank ID of the whole genome sequence of the virus that is the centroid sequence of the cluster. Column 2, nucleotides of CDS provides the nucleotides corresponding to the CDS of the gene in the whole genome. Column 3, Full Gene Name, provides the full name of the gene including Genbank ID, virus species, strain, and protein name. Column 4, Sequence, provides the amino acid sequence of the gene. Column 5, #Sequences/Cluster, provides the number of sequences that cluster with this centroid sequence. Column 6 provides the SEQ ID numbers for the described sequences.

TABLE 1
Exemplary Henipavirus protein G sequences

| Gen | | | | SEQ |
|---|---|---|---|---|
| bank | Nucleotides | Full sequence | | ID |
| ID | of CDS | ID | Sequence | NO |
| AFO | 8913- | gb: AF017149\| | MMADSKLVSLNNNLSGKIKDQGKVIKNYYGTMDIK | 4 |
| 171 | 10727 | Organism: | KINDGLLDSKILGAFNTVIALLGSIIIIVMNIMIIQNYTR | |
| 49 | | Hendra | TTDNQALIKESLQSVQQQIKALTDKIGTEIGPKVSLID | |
| | | virus\|Strain | TSSTITIPANIGLLGSKISQSTSSINENVNDKCKFTLPPL | |
| | | Name: | KIHECNISCPNPLPFREYRPISQGVSDLVGLPNQICLQ | |
| | | UNKNOWN- | KTTSTILKPRLISYTLPINTREGVCITDPLLAVDNGFFA | |
| | | AF017149\| | YSHLEKIGSCTRGIAKQRIIGVGEVLDRGDKVPSMF | |
| | | Protein | MTNVWTPPNPSTIHHCSSTYHEDFYYTLCAVSHVG | |
| | | Name: | DPILNSTSWTESLSLIRLAVRPKSDSGDYNQKYIAITK | |
| | | glycoprotein\| | VERGKYDKVMPYGPSGIKQGDTLYFPAVGFLPRTEF | |
| | | Gene Symbol: G | QYNDSNCPIIHCKYSKAENCRLSMGVNSKSHYILRSG | |
| | | | LLKYNLSLGGDIILQFIEIADNRLTIGSPSKIYNSLGQP | |
| | | | VFYQASYSWDTMIKLGDVDTVDPLRVQWRNNSVI | |
| | | | SRPGQSQCPRFNVCPEVCWEGTYNDAFLIDRLNW | |
| | | | VSAGVYLNSNQTAENPVFAVFKDNEILYQVPLAEDD | |
| | | | TNAQKTITDCFLLENVIWCISLVEIYDTGDSVIRPKLF | |
| | | | AVKIPAQCSES | |
| AF2 | 8943- | gb: AF212302\| | MPAENKKVRFENTTSDKGKIPSKVIKSYYGTMDIKKI | 3 |
| 123 | 10751 | Organism: | NEGLLDSKILSAFNTVIALLGSIVIIVMNIMIIQNYTRS | |
| 02 | | Nipah | TDNQAVIKDALQGIQQQIKGLADKIGTEIGPKVSLID | |
| | | virus\|Strain | TSSTITIPANIGLLGSKISQSTASINENVNEKCKFTLPPL | |
| | | Name:UNKNOWN- | KIHECNISCPNPLPFREYRPQTEGVSNLVGLPNNICL | |
| | | AF212302\| | QKTSNQILKPKLISYTLPVVGQSGTCITDPLLAMDEG | |
| | | Protein | YFAYSHLERIGSCSRGVSKQRIIGVGEVLDRGDEVPS | |
| | | Name: | LFMTNVWTPPNPNTVYHCSAVYNNEFYYVLCAVST | |
| | | attachment | VGDPILNSTYWSGSLMMTRLAVKPKSNGGGYNQH | |
| | | glycoprotein\| | QLALRSIEKGRYDKVMPYGPSGIKQGDTLYFPAVGF | |
| | | Gene | LVRTEFKYNDSNCPITKCQYSKPENCRLSMGIRPNSH | |
| | | Symbol:G | YILRSGLLKYNLSDGENPKVVFIEISDQRLSIGSPSKIY | |
| | | | DSLGQPVFYQASFSWDTMIKFGDVLTVNPLVVNW | |
| | | | RNNTVISRPGQSQCPRFNTCPEICWEGVYNDAFLID | |
| | | | RINWISAGVFLDSNQTAENPVFTVFKDNEILYRAQL | |
| | | | ASEDTNAQKTITNCFLLKNKIWCISLVEIYDTGDNVIR | |
| | | | PKLFAVKIPEQCT | |
| JQ0 | 8170- | gb: JQ001776: | MLSQLQKNYLDNSNQQGDKMNNPDKKLSVNFNP | 5 |
| 017 | 10275 | 8170-10275\| | LELDKGQKDLNKSYYVKNKNYNVSNLLNESLHDIKF | |
| 76 | | Organism: | CIYCIFSLLIIITIINIITISIVITRLKVHEENNGMESPNLQ | |
| | | Cedar | SIQDSLSSLTNMINTEITPRIGILVTATSVTLSSSINYVG | |
| | | virus\|Strain | TKTNQLVNELKDYITKSCGFKVPELKLHECNISCADP | |
| | | Name: CG1a\| | KISKSAMYSTNAYAELAGPPKIFCKSVSKDPDFRLKQI | |
| | | Protein | DYVIPVQQDRSICMNNPLLDISDGFFTYIHYEGINSC | |
| | | Name: | KKSDSFKVLLSHGEIVDRGDYRPSLYLLSSHYHPYSM | |
| | | attachment | QVINCVPVTCNQSSFVFCHISNNTKTLDNSDYSSDE | |
| | | glycoprotein\| | YYITYFNGIDRPKTKKIPINNMTADNRYIHFTFSGGG | |
| | | Gene | GVCLGEEFIIPVTTVINTDVFTHDYCESFNCSVQTGK | |
| | | Symbol:G | SLKEICSESLRSPTNSSRYNLNGIMIISQNNMTDFKIQ | |
| | | | LNGITYNKLSFGSPGRLSKTLGQVLYYQSSMSWDTY | |
| | | | LKAGFVEKWKPFTPNWMNNTVISRPNQGNCPRYH | |
| | | | KCPEICYGGTYNDIAPLDLGKDMYVSVILDSDQLAE | |
| | | | NPEITVFNSTTILYKERVSKDELNTRSTTTSCFLFLDEP | |
| | | | WCISVLETNRFNGKSIRPEIYSYKIPKYC | |
| NC_ | 9117- | gb: NC_025256: | MPQKTVEFINMNSPLERGVSTLSDKKTLNQSKITKQ | 6 |
| 025 | 11015 | 9117-11015\| | GYFGLGSHSERNWKKQKNQNDHYMTVSTMILEILV | |
| 256 | | Organism: | VLGIMFNLIVLTMVYYQNDNINQRMAELTSNITVLN | |
| | | Bat | LNLNQLTNKIQREIIPRITLIDTATTITIPSAITYILATLTT | |
| | | Paramyxovirus | RISELLPSINQKCEFKTPTLVLNDCRINCTPPLNPSDG | |
| | | Eid_hel/GH- | VKMSSLATNLVAHGPSPCRNFSSVPTIYYYRIPGLYN | |
| | | M74a/GHA/20 | RTALDERCILNPRLTISSTKFAYVHSEYDKNCTRGFKY | |
| | | 09\|Strain | YELMTFGEILEGPEKEPRMFSRSFYSPTNAVNYHSCT | |
| | | Name: BatPV/ | PIVTVNEGYFLCLECTSSDPLYKANLSNSTFHLVILRH | |
| | | Eid_hel/GH- | NKDEKIVSMPSFNLSTDQEYVQIIPAEGGGTAESGN | |
| | | M74a/GHA/20 | LYFPCIGRLLHKRVTHPLCKKSNCSRTDDESCLKSYYN | |
| | | 09\|Protein | QGSPQHQVVNCLIRIRNAQRDNPTWDVITVDLTNT | |
| | | Name: glyco- | YPGSRSRIFGSFSKPMLYQSSVSWHTLLQVAEITDLD | |
| | | protein\|Gene | KYQLDWLDTPYISRPGGSECPFGNYCPTVCWEGTY | |
| | | Symbol: G | NDVYSLTPNNDLFVTVYLKSEQVAENPYFAIFSRDQI | |
| | | | LKEFPLDAWISSARTTTISCFMFNNEIWCIAALEITRL | |
| | | | NDDIIRPIYYSFWLPTDCRTPYPHTGKMTRVPLRSTY | |
| | | | NY | |
| NC_ | 8716- | gb: NC_025352: | MATNRDNTITSAEVSQEDKVKKYYGVETAEKVADSI | 7 |
| 025 | 11257 | 8716-11257\| | SGNKVFILMNTLLILTGAIITITLNITNLTAAKSQQNM | |
| 352 | | Organism: | LKIIQDDVNAKLEMFVNLDQLVKGEIKPKVSLINTAV | |
| | | Mojiang | SVSIPGQISNLQTKFLQKYVYLEESITKQCTCNPLSGIF | |
| | | virus\|Strain | PTSGPTYPPTDKPDDDTTDDDKVDTTIKPIEYPKPDG | |
| | | Name: Tongguan1\| | CNRTGDHFTMEPGANFYTVPNLGPASSNSDECYTN | |
| | | Protein | PSFSIGSSIYMFSQEIRKTDCTAGEILSIQIVLGRIVDK | |
| | | Name: | GQQGPQASPLLVWAVPNPKIINSCAVAAGDEMG | |
| | | attachment | WVLCSVTLTAASGEPIPHMFDGFWLYKLEPDTEVVS | |
| | | glycoprotein\| | YRITGYAYLLDKQYDSVFIGKGGGIQKGNDLYFQMY | |
| | | Gene | GLSRNRQSFKALCEHGSCLGTGGGGYQVLCDRAV | |
| | | Symbol: G | MSFGSEESLITNAYLKVNDLASGKPVIIGQTFPPSDSY | |
| | | | KGSNGRMYTIGDKYGLYLAPSSWNRYLRFGITPDIS | |
| | | | VRSTTWLKSQDPIMKILSTCTNTDRDMCPEICNTRG | |
| | | | YQDIFPLSEDSEYYTYIGITPNNGGTKNFVAVRDSDG | |
| | | | HIASIDILQNYYSITSATISCFMYKDEIWCIAITEGKKQ | |
| | | | KDNPQRIYAHSYKIRQMCYNMKSATVTVGNAKNITI | |
| | | | RRY | |

In some embodiments, at least one G protein has a sequence set forth in any of SEQ ID NOS: 3-7 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 3-7.

In particular embodiments, the paramyxovirus envelope attachment protein (e.g., G protein) or functionally active variant or biologically active portion is a protein that retains fusogenic activity in conjunction with a paramyxovirus fusion protein (e.g., F protein), such as a NiV-F protein described herein. Fusogenic activity includes the activity of the paramyxovirus envelope attachment protein (e.g., G protein) in conjunction with a paramyxovirus fusion protein (e.g., F protein) to promote or facilitate fusion of two membrane lumens, such as the lumen of the targeted viral particle having embedded in its lipid bilayer a paramyxovirus fusion protein (e.g., F protein) and paramyxovirus envelope attachment protein (e.g., G protein), and a cytoplasm of a target cell, e.g. a cell that contains a surface receptor or molecule that is recognized or bound by the targeted envelope protein. In some embodiments, the paramyxovirus fusion protein (e.g., F protein) and the paramyxovirus envelope attachment protein (e.g., G protein) are from the same paramyxovirus species (e.g., the same Henipavirus species such as NiV-G and NiV-F).

In some embodiments, at least one G protein or the functionally active variant or biologically active portion thereof binds to Ephrin B2 or Ephrin B3. In some embodiments, the G protein is a variant G protein, such as a truncated G protein as described and retains binding to Ephrin B2 or B3. Reference to retaining binding to Ephrin B2 or B3 includes binding that is similar to the level or degree of binding of the corresponding wild-type G protein, such as set forth in SEQ ID NO: 3-7, such as at least 50%, at least 60%, at least 70%, at least 80% or at least 90% of the binding of the wild-type G protein.

In some embodiments, also provided are any of the provided variant NiV-G proteins that are re-targeted compared to the native tropism of NiV-G. For instance, mutations in NiV-G that completely abrogate ephrinB2 and B3 binding, but that do not impact the association of this NiV-G with NiV-F, have been identified (Aguilar, et al. J Biol Chem. 2009; 284 (3): 1628-1635.; Weise et al. J Virol. 2010; 84 (15): 7634-764; Negrete et al., J Virol. 2007; 81 (19): 10804-10814; Negrete et al. PLOS Pathog. 2006; Guillaume et al., J. Virol 2006, 80 (15) 7546-7554). Thus, in provided aspects, a variant NiV-G protein provided herein may further contain a mutation in its extracellular domain to reduce or abrogate binding to Ephrin B2 and/B3. In some embodiments, the mutations can include one or more of mutations E501A, W504A, Q530A and E533A, with reference to numbering of wild-type NiV-G set forth in SEQ ID NO:8. In some embodiments, any of the provided variant NiV-G proteins may also be linked or fused to a binding molecule for targeted binding to a target molecule of interest. In some embodiments, the variant G protein is a fusion of a binding molecule with variant NiV-G, including a NiV-G with mutations to abrogate Ephrin B2 and/or Ephrin B3 binding. This could allow for altered G protein tropism allowing for targeting of other desired cell types that are not ephrinB2+ through the addition of the binding molecule directed against a different cell surface molecule.

In some embodiments, the paramyxovirus envelope attachment protein, the first paramyxovirus envelope attachment protein, and/or the second paramyxovirus envelope attachment protein is a variant G protein that exhibits reduced binding for the native binding partner of a wild-type G protein. In some embodiments, the first paramyxovirus envelope attachment protein, and/or the second paramyxovirus envelope attachment protein and/or the third paramyxovirus envelope attachment protein is a variant G protein that exhibits reduced binding for the native binding partner of a wild-type G protein. In some embodiments, the first paramyxovirus envelope attachment protein, and/or the second paramyxovirus envelope attachment protein and/or the third paramyxovirus envelope attachment protein and/or the fourth paramyxovirus envelope attachment protein is a variant G protein that exhibits reduced binding for the native binding partner of a wild-type G protein. In some embodiments, the first paramyxovirus envelope attachment protein, and/or the second paramyxovirus envelope attachment protein and/or the third paramyxovirus envelope attachment protein and/or the fourth paramyxovirus envelope attachment protein and/or the fifth paramyxovirus envelope attachment protein, and/or one or more additional paramyxovirus envelope attachment proteins is a variant G protein that exhibits reduced binding for the native binding partner of a wild-type G protein. In some embodiments, the variant G protein or the biologically active portion thereof is a variant of wild-type NiV-G and exhibits reduced binding to one or both of the native binding partners Ephrin B2 or Ephrin B3. In some embodiments, the variant G-protein or the biologically active portion, such as a variant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations can improve transduction efficiency. In some embodiments, the mutations allow for specific targeting of other desired cell types that are not Ephrin B2 or Ephrin B3. In some embodiments, the mutations result in at least the partial inability to bind at least one natural receptor, such as to reduce the binding to at least one of Ephrin B2 or Ephrin B3. In some embodiments, the mutations described herein interfere with natural receptor recognition.

In some embodiments, at least one G protein contains one or more amino acid substitutions in a residue that is involved in the interaction with one or both of Ephrin B2 and Ephrin B3. In some embodiments, the amino acid substitutions correspond to mutations E501A, W504A, Q530A and E533A with reference to numbering set forth in SEQ ID NO:3. In some embodiments, at least one G protein is a variant G protein containing one or more amino acid substitutions selected from the group consisting of E501A, W504A, Q530A and E533A with reference to numbering set forth in SEQ ID NO:3. In some embodiments, at least one G protein is a variant G protein that contains one or more amino acid substitutions elected from the group consisting of E501A, W504A, Q530A and E533A with reference to SEQ ID NO:3 and is a biologically active portion thereof containing an N-terminal truncation.

In some embodiments, the NiV-G is a variant NiV-G proteins that contain an altered cytoplasmic tail compared to native NiV-G (e.g., SEQ ID NO:8) that are or can be incorporated into a viral particle, such as a viral particle, including a lentiviral particle or lentiviral-like particle. The cytoplasmic tail of NiV-G corresponds to amino acids 1-45 of SEQ ID NO: 8. In some cases, it is understood that the N-terminal methionine of NiV-G, or a variant NiV-G, as described herein can be cleaved and the cytoplasmic tail lacks an initial N-terminal methionine. For instance, in some embodiments, the cytoplasmic tail of wild-type NiV-G may correspond to amino acids 2-45 of SEQ ID NO:8, and the variant NiV-G protein contains a cytoplasmic tail that is altered compared to amino acids 2-45 of SEQ ID NO:8. In some embodiments, the variant NiV-G contains a modified cytoplasmic tail in which the native cytoplasmic tail is truncated or is replaced by a heterologous cytoplasmic tail.

Non-limiting examples of variant NiV-G proteins, including truncated NiV-G or NiV-G with an altered or modified cytoplasmic tail, are described in WO2013148327, WO2017182585, or PCT/US2022/081872. Further exemplary variant NiV-G proteins are described in Bender et al. 2016 PLOS Pathol 12 (6): e1005641.

In some embodiments, at least one G protein is a variant G protein that is a functionally active variant or biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference G protein sequence. In some embodiments, the reference G protein sequence is the wild-type sequence of a G protein or a biologically active portion thereof. In some embodiments, at least one functionally active variant or the biologically active portion thereof is a variant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein or biologically active portion thereof. In some embodiments, the wild-type G protein has the sequence set forth in any one of SEQ ID NOS: 3-7.

In some embodiments, at least one G protein is a variant G protein that is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein. In particular embodiments, the truncation is an N-terminal truncation of all or a portion of the cytoplasmic domain. In some embodiments, at least one variant G protein is a biologically active portion that is truncated and lacks up to 49 contiguous amino acid residues at or near the N-terminus of the wild-type G protein, such as a wild-type G protein set forth in any one of SEQ ID NOS: 3-7. In some embodiments, at least one variant G protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type G protein.

In some embodiments, at least one G protein is a wild-type Nipah virus G (NiV-G) protein or a Hendra virus G protein, or is a functionally active variant or biologically active portion thereof. In some embodiments, at least one G protein is a NiV-G protein that has the sequence set forth in SEQ ID NO:1, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:3.

In some embodiments, at least one G protein is a variant NiV-G that comprises a modified cytoplasmic tail which comprises a truncated cytoplasmic tail from a glycoprotein from the same Nipah virus. In some embodiments, the variant NiV-G contains a modified cytoplasmic tail in which at least a part of the native cytoplasmic tail (e.g., corresponding to amino acids 1-45 of SEQ ID NO:8) is a truncated portion thereof from a glycoprotein from Nipah Virus. In some embodiments, the cytoplasmic tail is a truncated portion thereof that is at least 5 amino acids in length. from or from about 5-44, from or from about 5-40, from or from about 5-30, from or from about 5-20, from or from about 5-10, from or from about 10-44, from or from about 10-40, from or from about 10-30, from or from about 10-20, from or from about 20-44, from or from about 20-40, from or from about 20-30, from or from about 30-44, from or from about 30-40, from or from about 40-44amino acids in length. In some embodiments, the truncated portion thereof is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 or 44 amino acids in length. In some embodiments, the variant NiV-G has a cytoplasmic tail that is a truncated NiV-G cytoplasmic tail.

In some embodiments, the truncated NiV-G cytoplasmic tail has a deletion of up to 40, up to 35, up to 30, up to 29, up to 28, up to 27, up to 26, up to 25, up to 24, up to 23, up to 22, up to 21, up to 20, up to 19, up to 18, up to 17, up to 16, up to 15, or up to 14 contiguous amino acid residues at or near the N-terminus of the wild-type NiV-G cytoplasmic tail set forth in SEQ ID NO: 9.

In some embodiments, the cytoplasmic tail of NiV-G is set forth in SEQ ID NO: 10. In some embodiments, the truncated NiV-G cytoplasmic tail has a deletion of up to 40, up to 35, up to 30, up to 29, up to 28, up to 27, up to 26, up to 25, up to 24, up to 23, up to 22, up to 21, up to 20, up to 19, up to 18, up to 17, up to 16, up to 15, or up to 14 contiguous amino acid residues at or near the N-terminus of the wild-type NiV-G cytoplasmic tail set forth in SEQ ID NO: 10.

In some embodiments, the variant NiV-G has a deletion of between 5 and 41 contiguous amino acid residues at or near the N-terminus of the wild-type NiV-G protein cytoplasmic tail set forth in SEQ ID NO: 10. In some embodiments, the variant NiV-G has a deletion of between 26 and 40 contiguous amino acid residues at or near the N-terminus of the wild-type NiV-G protein cytoplasmic tail set forth in SEQ ID NO: 10.

In some embodiments, at least one G protein is a variant NiV-G protein that is a biologically active portion of a wild-type NiV-G. In some embodiments, the biologically active portion is an N-terminally truncated fragment. In some embodiments, the variant NiV-G protein is truncated and lacks up to 5 contiguous amino acid residues at or near the N-terminus of the wild-type NiV-G protein, such as compared to wild-type NiV-G set forth in SEQ ID NO: 3. In some embodiments, the variant NiV-G protein is truncated and lacks up to 10 contiguous amino acid residues at or near the N-terminus of the wild-type NiV-G protein, such as compared to wild-type NiV-G set forth in SEQ ID NO: 3. In some embodiments, the variant NiV-G protein is truncated and lacks up to 15 contiguous amino acid residues at or near the N-terminus of the wild-type NiV-G protein, such as compared to wild-type NiV-G set forth in SEQ ID NO: 3. n some embodiments, the variant NiV-G protein is truncated and lacks up to 20 contiguous amino acid residues at or near the N-terminus of the wild-type NiV-G protein, such as compared to wild-type NiV-G set forth in SEQ ID NO: 3. In some embodiments, the variant NiV-G protein is truncated and lacks up to 25 contiguous amino acid residues at or near the N-terminus of the wild-type NiV-G protein, such as compared to wild-type NiV-G set forth in SEQ ID NO: 3. In some embodiments, the variant NiV-G protein is truncated and lacks up to 30 contiguous amino acid residues at or near the N-terminus of the wild-type NiV-G protein, such as compared to wild-type NiV-G set forth in SEQ ID NO: 3. In some embodiments, the variant NiV-G protein is truncated and lacks up to 35 contiguous amino acid residues at or near the N-terminus of the wild-type NiV-G protein, such as compared to wild-type NiV-G set forth in SEQ ID NO: 3. In some embodiments, the variant NiV-G protein (also called variant NiV-G) contains an N-terminal methionine.

In some embodiments, the variant NiV-G has a cytoplasmic tail deletion of amino acid residues 2-41, 2-40, 2-39, 2-38, 2-37, 2-36, 2-34, 2-35, 2-33, 2-32, 2-31, 2-30, 2-29, 2-28, 2-27, 2-26, 2-25, 2-22, 2-21, 2-16, 2-11, or 2-5 of SEQ ID NO:10.

In some embodiments, the variant NiV-G contains a modified cytoplasmic tail in which at least a part of the native cytoplasmic tail (e.g., corresponding to amino acids 1-45 of SEQ ID NO:8) is replaced by a heterologous cytoplasmic tail or a truncated portion thereof from a glycoprotein from another virus from another virus or viral-associated protein. In some embodiments, the replaced cytoplasmic tail is a heterologous cytoplasmic tail or a truncated portion thereof that is at least 5 amino acids in length. In some embodiments, the replaced heterologous cytoplasmic tail or a truncated portion thereof is from or from about 5-180 amino acids in length, such as from or from about 5-150, from or from about 5-100, from or from about 5-75, from or from about 5-50, from or from about 5-40, from or from about 5-30, from or from about 5-20, from or from about 5-10, from or from about 10-150, from or from about 10-100, from or from about 10-75, from or from about 10-50, from or from about 10-40, from or from about 10-30, from or from about 10-20, from or from about 20-150, from or from about 20-100, from or from about 20-75, from or from about 20-50, from or from about 20-40, from or from about 20-30, from or from about 30-150, from or from about 30-100, from or from about 30-75, from or from about 30-50, from or from about 30-40, from or from about 40-150, from or from about 40-100, from or from about 40-75, from or from about 40-50, from or from about 50-150, from or from about 50-100, from or from about 50-75, from or from about 75-150, from or from about 75-100 or from or from about 100-150 amino acids in length. In some embodiments, the replaced heterologous cytoplasmic tail or a truncated portion thereof is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids in length. In some embodiments, the heterologous cytoplasmic tail or the truncated portion thereof is directly linked to the N-terminus of the sequence set forth in SEQ ID NO: 11. In some embodiments, the heterologous cytoplasmic tail or the truncated portion thereof is directly linked to the N-terminus of the sequence set forth in SEQ ID NO: 12.

In some embodiments, the heterologous cytoplasmic tail is a cytoplasmic tail or a truncated portion thereof from a glycoprotein from another virus, such as a paramyxovirus, a retrovirus, a filovirus, a rhabdovirus or an arenavirus. In some embodiments, the virus is a paramyxovirus other than a Nipah virus. For instance, the virus is a measles virus, Bat paramyxovirus, Cedar Virus, Canine Distemper Virus, Sendai virus, Hendra virus, Human Parainfluenza virus, or Newcastle Disease virus.

In some embodiments, the virus is a retrovirus. For instance, the virus may be a baboon endogenous virus (BaEV), Gibbon Ape Leukemia virus (GaLV), murine leukemia virus, or human immunodeficiency virus 1 (HIV-1). In some embodiments, the replaced heterologous cytoplasmic tail is the native cytoplasmic tail or a truncated portion of the native cytoplasmic tail of another virusIn some embodiments, the variant NiV-G contains mutations in the extracellular domain that reduce or abrogate binding to an Ephrin B2 or B3 corresponding to one or more of E501A, W504A, Q530A and E533A, with numbering of residues as set forth SEQ ID NO:3.

In some embodiments, at least one variant NiV-G protein is truncated and lacks up to amino acid 34 at or near the N-terminus of the wild-type NiV-G protein, such as compared to wild-type NiV-G set forth in SEQ ID NO: 3. In some embodiments, the variant NiV-G protein (also called variant NiV-G) contains an N-terminal methionine. In some embodiments, the variant NiV-G protein lacks amino acids 2-34 as compared to wild-type NiV-G set forth in SEQ ID NO: 1. In some embodiments, the NiV-G has the sequence set forth in SEQ ID NO:13.

In particular embodiments, at least one G protein has the sequence of amino acids set forth in SEQ ID NO: 13, or is a functionally active variant thereof or a biologically active portion thereof that retains binding and/or fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 13 and retains fusogenic activity in conjunction with a variant NiV-F protein as described. In some embodiments, at least one G protein is a variant G protein that comprises the amino acid sequence of SEQ ID NO: 13.

In some embodiments, any of the provided viral particles (lentiviral vectors) may also contain an F protein, such as a NiV-F protein, such as a full-length NiV-F protein or a biologically active portion thereof or a variant thereof. For instance, also provided herein are viral particles or viral-like particles, such as lentiviral particles or lentiviral-like particles, that are pseudotyped with any of the provided variant NiV-G proteins and a NiV-F protein, such as a full-length NiV-F protein or a biologically active portion or a variant thereof. Exemplary NiV-F proteins are further described herein.

In particular embodiments, the paramyxovirus envelope attachment protein, first paramyxovirus envelope attachment protein and/or second paramyxovirus envelope attachment protein, such as at least one G protein or functionally active variant or biologically active portion thereof, is a protein that retains fusogenic activity in conjunction with other retargeted attachment proteins, such as more than one G protein expressed as a multimer on the lipid bi-layer. In particular embodiments, the first paramyxovirus envelope attachment protein and/or the second paramyxovirus envelope attachment protein and/or the third paramyxovirus envelope attachment protein, such as at least one G protein or functionally active variant or biologically active portion thereof, is a protein that retains fusogenic activity in conjunction with other retargeted attachment proteins, such as more than one G protein expressed as a multimer on the lipid bi-layer. In particular embodiments, the first paramyxovirus envelope attachment protein and/or the second paramyxovirus envelope attachment protein and/or the third paramyxovirus envelope attachment protein and/or the fourth paramyxovirus envelope attachment protein, such as at least one G protein or functionally active variant or biologically active portion thereof, is a protein that retains fusogenic activity in conjunction with other retargeted attachment proteins, such as more than one G protein expressed as a multimer on the lipid bi-layer. In particular embodiments, the first paramyxovirus envelope attachment protein and/or the second paramyxovirus envelope attachment protein and/or the third paramyxovirus envelope attachment protein and/or the fourth paramyxovirus envelope attachment protein and/or one or more additional paramyxovirus envelope attachment proteins, such as at least one G protein or functionally active variant or biologically active portion thereof, is a protein that retains fusogenic activity in conjunction with other retargeted attachment proteins, such as more than one G protein expressed as a multimer on the lipid bi-layer. Fusogenic activity includes the activity of the paramyxovirus envelope attachment protein in conjunction with a protein that is a paramyxovirus fusion protein (e.g., an F protein) to promote or facilitate fusion of two membrane lumens, such as the lumen of the targeted viral particle having embedded in its lipid bilayer at least two paramyxovirus envelope attachment protein and paramyxovirus fusion protein (e.g., F and G proteins), and a cytoplasm of a target cell, e.g. a cell that contains a surface receptor or molecule that is recognized or bound by the targeted envelope protein.

Reference to retaining fusogenic activity includes activity of a viral particle (e.g. lentiviral vector) containing at least two paramyxovirus envelope attachment protein and paramyxovirus fusion protein (e.g., F and G proteins) that is between at or about 10% and at or about 150% or more of the level or degree of binding of a reference viral particle (e.g. lentiviral vector) that is similar, such as contains the same variant NiV-F, but that contains the corresponding wild-type G protein, such as set forth in SEQ ID NO: 1. For instance, a viral particle (e.g. lentiviral vector) that retains fusogenic activity has at least or at least about 10% of the level or degree of fusogenic activity of the reference viral particle that is similar (such as contains the same variant NiV-F) but that contains the corresponding wild-type G protein, such as at least or at least about 15% of the level or degree of fusogenic activity, at least or at least about 20% of the level or degree of fusogenic activity, at least or at least about 25% of the level or degree of fusogenic activity, at least or at least about 30% of the level or degree of fusogenic activity, at least or at least about 35% of the level or degree of fusogenic activity, at least or at least about 40% of the level or degree of fusogenic activity, at least or at least about 45% of the level or degree of fusogenic activity, at least or at least about 50% of the level or degree of fusogenic activity, at least or at least about 55% of the level or degree of fusogenic activity, at least or at least about 60% of the level or degree of fusogenic activity, at least or at least about 65% of the level or degree of fusogenic activity, at least or at least about 70% of the level or degree of fusogenic activity, at least or at least about 75% of the level or degree of fusogenic activity, at least or at least about 80% of the level or degree of fusogenic activity, at least or at least about 85% of the level or degree of fusogenic activity, at least or at least about 90% of the level or degree of fusogenic activity, at least or at least about 95% of the level or degree of fusogenic activity, at least or at least about 100% of the level or degree of fusogenic activity, or at least or at least about 120% of the level or degree of fusogenic activity.

Reference to retaining fusogenic activity includes activity of a viral particle (e.g. lentiviral vector) containing at least two paramyxovirus envelope attachment protein and paramyxovirus fusion protein (e.g., F and G proteins) that is between at or about 10% and at or about 150% or more of the level or degree of binding of a reference viral particle (e.g. lentiviral vector) that is similar, such as contains the same variant NiV-F, but that contains only one of the provided paramyxovirus envelope attachment proteins (e.g., G proteins). For instance, a viral particle (e.g. lentiviral vector) that retains fusogenic activity has at least or at least about 10% of the level or degree of fusogenic activity of the reference viral particle that is similar (such as contains the same variant NiV-F) but that contains only one of the provided paramyxovirus envelope attachment proteins, such as at least or at least about 15% of the level or degree of fusogenic activity, at least or at least about 20% of the level or degree of fusogenic activity, at least or at least about 25% of the level or degree of fusogenic activity, at least or at least about 30% of the level or degree of fusogenic activity, at least or at least about 35% of the level or degree of fusogenic activity, at least or at least about 40% of the level or degree of fusogenic activity, at least or at least about 45% of the level or degree of fusogenic activity, at least or at least about 50% of the level or degree of fusogenic activity, at least or at least about 55% of the level or degree of fusogenic activity, at least or at least about 60% of the level or degree of fusogenic activity, at least or at least about 65% of the level or degree of fusogenic activity, at least or at least about 70% of the level or degree of fusogenic activity, at least or at least about 75% of the level or degree of fusogenic activity, at least or at least about 80% of the level or degree of fusogenic activity, at least or at least about 85% of the level or degree of fusogenic activity, at least or at least about 90% of the level or degree of fusogenic activity, at least or at least about 95% of the level or degree of fusogenic activity, at least or at least about 100% of the level or degree of fusogenic activity, or at least or at least about 120% of the level or degree of fusogenic activity.

In some embodiments, the paramyxovirus G proteins are mutant Paramyxovirus G glycoproteins (e.g., variant Paramyxovirus G glycoproteins) comprising one or more amino acid mutations that result in decreased glycosylation of the protein. The one or more amino acid mutations, also called deglycosylation mutations, can be one or more amino acid substitutions (also referred to as mutations). Non-limiting examples of mutant Paramyxovirus G glycoproteins (e.g., variant Paramyxovirus G glycoproteins) comprising one or more amino acid mutations that result in decreased glycosylation of the protein are described in PCT Publication WO 2024/064838, which is hereby incorporated by reference in its entirety.

### B. Re-Targeted Fusogens

In some embodiments, a paramyxovirus envelope attachment protein, such as a G protein (e.g., NiV-G), is further attached or linked to a binding domain that binds to a target molecule to comprise a retargeted attachment protein. For instance, provided in some aspects is a viral particle that includes a targeted paramyxovirus envelope attachment proteins (e.g., a chimeric attachment G protein) containing any of the provided G proteins described above that is attached to a binding domain, in which the retargeted attachment protein (e.g., re-targeted G protein) is exposed on the surface of the targeted viral particle (e.g. lentiviral vector). In some of any of the provided embodiments, the viral particle comprises a retargeted attachment protein comprising (i) a paramyxovirus envelope attachment protein; and (ii) a targeting moiety directed to a first target molecule expressed on the surface of a target cell.

In some embodiments, each of the one or more of the paramyxovirus envelope attachment proteins, such as a G protein (e.g., NiV-G), is further attached or linked to targeting moiety, e.g., a binding domain or a binding agent, directed to a target molecule expressed on the surface of a target cell. The binding domain or binding agent can be any binding domain or binding agent described herein, e.g., in Section II. Accordingly, in some embodiments, the viral particle comprises one or more retargeted attachment proteins, wherein each of the one or more retargeted attachment proteins independently comprise: (i) a paramyxovirus envelope attachment protein; and (ii) a targeting moiety directed to a target molecule expressed on the surface of a target cell. The targeting moiety can be a binding domain or binding agent, such as any binding domain or any binding agent described herein.

In some embodiments, the envelope attachment protein is a retargeted attachment protein containing a henipavirus G protein or a biologically active portion thereof. In some embodiments, the envelope attachment proteins (e.g., G protein) may be retargeted by linkage to a targeting moiety, such as a binding molecule (e.g., antibody or antigen-binding fragment, e.g., sdAb or scFv) that binds to a target cell, such as a retargeted attachment protein. In some embodiments, the retargeted attachment protein and paramyxovirus fusion protein (e.g., G protein and a NiV-F protein provided herein) together exhibit fusogenic activity to a target cell, such as to deliver an exogenous agent or nucleic acid exogenous agent to the target cell.

In some embodiments, the viral particle comprises at least two retargeted attachment proteins comprising paramyxovirus envelope attachment proteins (e.g., G proteins), wherein at least one is retargeted by linkage to a targeting moiety, such as a binding molecule (e.g., antibody or antigen-binding fragment, e.g., sdAb or scFv) that binds to a target cell. In some embodiments, the viral particle comprises at least two retargeted attachment proteins comprising envelope attachment proteins (e.g., G proteins), wherein at least two are retargeted by linkage to a targeting moiety, such as a binding molecule (e.g., antibody or antigen-binding fragment, e.g., sdAb or scFv) that binds to a target cell.

In some embodiments, the first and second retargeted attachment proteins are retargeted by linkage to a targeting moiety, wherein the targeting moiety is directed to a target molecule expressed on the surface of a target cell. In some embodiments, the first and second retargeted attachment proteins are retargeted by linkage to a first and second targeting moiety, wherein the first and second targeting moiety are directed to the same target molecule expressed on the surface of a target cell. In some embodiments, the first and second retargeted attachment proteins are retargeted by linkage to a first and second targeting moiety, wherein the first and second targeting moiety are directed to a first and second target molecule expressed on the surface of a target cell that are different. In some embodiments, the targeting one or both of the first target molecule and the second target molecule does not activate or inhibit, induce a phenotype change (for example maturation and/or differentiation), induce proliferation, and/or induce apoptosis of said target cell.

In some embodiments, the viral particle comprises at least three retargeted attachment proteins comprising paramyxovirus envelope attachment proteins (e.g., G proteins), wherein at least one is retargeted by linkage to a targeting moiety, such as a binding molecule (e.g., antibody or antigen-binding fragment, e.g., sdAb or scFv) that binds to a target cell. In some embodiments, the viral particle comprises at least three retargeted attachment proteins comprising envelope attachment proteins (e.g., G proteins), wherein at least two are retargeted by linkage to a targeting moiety, such as a binding molecule (e.g., antibody or antigen-binding fragment, e.g., sdAb or scFv) that binds to a target cell. In some embodiments, the viral particle comprises at least three retargeted attachment proteins comprising envelope attachment proteins (e.g., G proteins), wherein at least three are retargeted by linkage to a targeting moiety, such as a binding molecule (e.g., antibody or antigen-binding fragment, e.g., sdAb or scFv) that binds to a target cell.

In some embodiments, the first, second, and third retargeted attachment proteins are retargeted by linkage to a targeting moiety, wherein the targeting moiety is directed to a target molecule expressed on the surface of a target cell. In some embodiments, the first, second, and third retargeted attachment proteins are retargeted by linkage to a first, second, and third targeting moiety, wherein the first and second targeting moiety, or the second and third targeting moiety, or the first and third targeting moiety, or the first, second, and third target moiety, are directed to the same target molecule expressed on the surface of a target cell. In some embodiments, the first, second, and third retargeted attachment proteins are retargeted by linkage to a first, second, and third targeting moiety, wherein the first, second, and third targeting moiety are directed to a first, second, and third target molecule expressed on the surface of a target cell that are different. In some embodiments, the targeting of one, two, or three of the first target molecule, the second target molecule, and the third target molecule does not activate or inhibit, induce a phenotype change (for example maturation and/or differentiation), induce proliferation, and/or induce apoptosis of said target cell.

In some embodiments, the lentiviral particle comprises at least four or at least five retargeted attachment proteins comprising paramyxovirus envelope attachment proteins (e.g., G proteins), wherein at least one, at least two, at least three, or at least four is retargeted by linkage to a targeting moiety, such as a binding molecule (e.g. antibody or antigen-binding fragment, e.g. sdAb or scFv) that binds to a target cell. In some embodiments, the lentiviral particle comprises at least four or at least five retargeted attachment proteins comprising envelope attachment proteins (e.g., G proteins), wherein at least two or at least three are retargeted by linkage to a targeting moiety, such as a binding molecule (e.g., antibody or antigen-binding fragment, e.g., sdAb or scFv) that binds to a target cell. In some embodiments, the lentiviral particle comprises at least four or at least five retargeted attachment proteins comprising envelope attachment proteins (e.g., G proteins), wherein at least four or at least five are retargeted by linkage to a targeting moiety, such as a binding molecule (e.g., antibody or antigen-binding fragment, e.g., sdAb or scFv) that binds to a target cell.

In some embodiments, the first, second, third, and fourth retargeted attachment proteins are retargeted by linkage to a targeting moiety, wherein the targeting moiety is directed to a target molecule expressed on the surface of a target cell. In some embodiments, the first, second, third, and fourth retargeted attachment proteins are retargeted by linkage to a first, second, third, and fourth targeting moiety, wherein at least two or at least three of the first, second, third, and fourth targeting moiety are directed to the same target molecule expressed on the surface of a target cell. In some embodiments, at least two of the first, second, third, and fourth retargeted attachment proteins are retargeted by linkage to a first, second, third, and fourth targeting moiety, wherein the first, second, third, and fourth targeting moiety are directed to a first, second, third, and fourth target molecule expressed on the surface of a target cell that are different. In some embodiments, the targeting of one, two, three, or four of the first target molecule, the second target molecule, the third target molecule, and fourth target molecule, does not activate or inhibit, induce a phenotype change (for example maturation and/or differentiation), induce proliferation, and/or induce apoptosis of said target cell.

In some embodiments, the first, second, third, fourth, and fifth retargeted attachment proteins are retargeted by linkage to a targeting moiety, wherein the targeting moiety is directed to a target molecule expressed on the surface of a target cell. In some embodiments, the first, second, third, fourth, and fifth retargeted attachment proteins are retargeted by linkage to a first, second, third, fourth, and fifth targeting moiety, wherein at least two or at least three of the first, second, third, fourth, and fifth targeting moiety are directed to the same target molecule expressed on the surface of a target cell. In some embodiments, the first, second, third, fourth, and fifth retargeted attachment proteins are retargeted by linkage to a first, second, third, fourth, and fifth targeting moiety, wherein at least two of the first, second, third, fourth, and fifth targeting moiety are directed to a first, second, third, fourth, and fifth target molecule expressed on the surface of a target cell that are different. In some embodiments, the targeting of one, two, three, four, or five of the first target molecule, the second target molecule, the third target molecule, fourth target molecule, and fifth target molecule, does not activate or inhibit, induce a phenotype change (for example maturation and/or differentiation), induce proliferation, and/or induce apoptosis of said target cell.

In some embodiments, the paramyxovirus retargeted attachment protein is a targeted envelope protein containing a G protein provided herein. In some embodiments the paramyxovirus retargeted attachment protein comprises at least one envelope attachment proteins (e.g., G protein) that is any of those described herien, including NiV-G proteins with cytoplasmic domain modifications, truncated NiV-G cytoplasmic tails, or modified NiV-G cytoplasmic tails.

In some embodiments, the retargeted attachment protein comprises (i) a paramyxovirus envelope attachment protein; and (ii) a targeting moiety directed to a first target molecule expressed on the surface of a target cell. In some embodiments, the retargeted attachment protein, e.g., each of one or more of the first, second, third, fourth, fifth, or additional retargeted attachment protein, comprises (i) a paramyxovirus envelope attachment protein, and (ii) a targeting moiety directed to a target molecule expressed on the surface of a target cell. In some embodiments, the targeting moiety is a binding domain, such as any of the binding domains or binding agents described herein, e.g., a T cell binding domain or an HSC binding domain. In some embodiments, the binding domain can be any agent that binds to a cell surface molecule on a target cells. In some embodiments, the binding domain can be an antibody or an antibody portion or fragment. In some embodiments, the binding domain is a single domain antibody (sdAb). In some embodiments, the binding domain is a single chain variable fragment (scFv). The binding domain can be linked directly or indirectly to the G protein. In particular embodiments, the binding domain is linked to the C-terminus (C-terminal amino acid) of the G protein or the biologically active portion thereof. The linkage can be via a peptide linker, such as a flexible peptide linker.

The retargeted attachment protein comprising a binding domain linked to at least one paramyxovirus envelope attachment protein may be modulated to have different binding strengths. For example, scFvs and antibodies with various binding strengths may be used to alter the fusion activity of the retargeted attachment proteins towards cells that display high or low amounts of the target antigen. For example DARPins with different affinities may be used to alter the fusion activity towards cells that display high or low amounts of the target antigen. Binding domains may also be modulated to target different regions on the target ligand, which will affect the fusion rate with cells displaying the target.

The binding domain may comprise a humanized antibody molecule, intact IgA, IgG, IgE or IgM antibody; bi- or multi-specific antibody (e.g., Zybodies®, etc.); antibody fragments such as Fab fragments, Fab′ fragments, F(ab′) 2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. A targeting moiety can also include an antibody or an antigen-binding fragment thereof (e.g., Fab, Fab′, F(ab′) 2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs).

The binding domain may comprise a humanized antibody molecule, intact IgA, IgG, IgE or IgM antibody; bi- or multi-specific antibody (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. A targeting moiety can also include an antibody or an antigen-binding fragment thereof (e.g., Fab, Fab′, F(ab′) 2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody or a T cell receptor (TCRs). In some embodiments, the binding domain does not comprise a ligand, a cytokine, or a chemokine,

In some embodiments, the binding domain is a single chain molecule. In some embodiments, the binding domain is a single domain antibody. In some embodiments, the binding domain is a single chain variable fragment. In particular embodiments, the binding domain contains an antibody variable sequence(s) that is human or humanized.

In some embodiments, the binding domain is a single domain antibody. In some embodiments, the single domain antibody can be human or humanized. In some embodiments, the single domain antibody or portion thereof is naturally occurring. In some embodiments, the single domain antibody or portion thereof is synthetic.

In some embodiments, the single domain antibodies are antibodies whose complementary determining regions are part of a single domain polypeptide. In some embodiments, the single domain antibody is a heavy chain only antibody variable domain. In some embodiments, the single domain antibody does not include light chains.

In some embodiments, the heavy chain antibody devoid of light chains is referred to as VHH. In some embodiments, the single domain antibody antibodies have a molecular weight of 12-15 kDa. In some embodiments, the single domain antibody antibodies include camelid antibodies or shark antibodies. In some embodiments, the single domain antibody molecule is derived from antibodies raised in Camelidae species, for example in camel, llama, dromedary, alpaca, vicuna and guanaco. In some embodiments, the single domain antibody is referred to as immunoglobulin new antigen receptors (IgNARs) and is derived from cartilaginous fishes. In some embodiments, the single domain antibody is generated by splitting dimeric variable domains of human or mouse IgG into monomers and camelizing critical residues.

In some embodiments, the single domain antibody can be generated from display libraries, e.g., phage display libraries. In some embodiments, the display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the display library is generated comprising antibody fragments of a non-immunized camelid. In some embodiments, single domain antibodies a library of human single domain antibodies is synthetically generated by introducing diversity into one or more scaffolds.

In some embodiments, the binding domain is a single domain antibody (sdAb). In some embodiments, the binding domain is a single chain variable fragment (scFv). The binding domain can be linked directly or indirectly to the paramyxovirus envelope attachment protein, first paramyxovirus envelope attachment protein, and/or second paramyxovirus envelope attachment protein (e.g., G protein). In particular embodiments, the binding domain is linked to the C-terminus (C-terminal amino acid) of the G protein or the biologically active portion thereof. The linkage can be via a peptide linker, such as a flexible peptide linker.

In some embodiments, the C-terminus of the binding domain is attached to the C-terminus of the G protein or biologically active portion thereof. In some embodiments, the N-terminus of the binding domain is exposed on the exterior surface of the lipid bilayer. In some embodiments, the N-terminus of the binding domain binds to a cell surface molecule of a target cell. In some embodiments, the binding domain specifically binds to a cell surface molecule present on a target cell. In some embodiments, the cell surface molecule is a protein, glycan, lipid or low molecular weight molecule. In some embodiments, the binding domain is one of any binding domains as described above.

In some embodiments, a binding domain (e.g., sdAb or one of any binding domains as described herein) binds to a cell surface antigen of a cell. In some embodiments, a cell surface antigen is characteristic of one type of cell. In some embodiments, a cell surface antigen is characteristic of more than one type of cell.

In some embodiments, the cell surface molecule of a target cell is an antigen or portion thereof. In some embodiments, the single domain antibody or portion thereof is an antibody having a single monomeric domain antigen binding/recognition domain that is able to bind selectively to a specific antigen. In some embodiments, the single domain antibody binds an antigen present on a target cell.

Exemplary cells include polymorphonuclear cells (also known as PMN, PML, PMNL, or granulocytes), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, immune effector cells, lymphocytes, macrophages, dendritic cells, natural killer cells, T cells, cytotoxic T lymphocytes, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes.

In some embodiments, the target cell is a cell of a target tissue. The target tissue can include liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g. hepatocyte), or a cardiac cell (e.g. cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g. a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoietic stem cell (HSC), a liver cell or a fully differentiated cell. In some embodiments, the target cell is a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell). In some embodiments, the first and second target molecules are present on the same target cell. In some embodiments, the first and second target molecules are present on different cells.

In some embodiments, the binding domain (e.g. sdAb) variable domain binds a cell surface molecule or antigen. In some embodiments, the cell surface molecule is ASGR1, ASGR2, TM4SF5, CD8, CD4, or low density lipoprotein receptor (LDL-R). In some embodiments, the cell surface molecule is ASGR1. In some embodiments, the cell surface molecule is ASGR2. In some embodiments, the cell surface molecule is TM4SF5. In some embodiments, the cell surface molecule is CD8. In some embodiments, the cell surface molecule is CD4. In some embodiments, the cell surface molecule is LDL-R.

In some embodiments, the target cell is a hematopoietic lineage cell. Reference to a “hematopoietic cell” includes blood cells, both from the myeloid and the lymphoid lineage. In particular, the term “hematopoietic cell” includes both undifferentiated or poorly differentiated cells, such as hematopoietic stem cells and progenitor cells, and differentiated cells such as T lymphocytes, B lymphocytes, or dendritic cells. In some embodiments, the hematopoietic cells are hematopoietic stem cells (HSCs), CD34+ progenitor cells, in particular peripheral blood CD34+ cells, very early progenitor CD34+ cells, B-cell CD19+ progenitors, myeloid progenitor CD13+ cells, T lymphocytes, B lymphocytes, monocytes, dendritic cells, cancer B cells in particular B-cell chronic lymphocytic leukemia (BCLL) cells and marginal zone lymphoma (MZL) B cells, or thymocytes.

As known from the skilled person, many hematopoietic cells are produced from bone marrow hematopoietic stem cells.

In some embodiments, a hematopoietic cell is a hematopoietic stem cell (HSC), which are cells able to replenish all blood cell types and to self-renew. Hematopoietic stem cells may be in particular defined as cells that keep the levels of myeloid, T cells, and B cells at robustly detectable levels (typically more than 1% of peripheral blood cells) for 16 weeks when injected into the circulation of a recipient mouse with a depleted hematopoietic system (Schroeder (2010) Cell Stem Cell 6:203-207).

In some embodiments, the hematopoietic cell is a “CD34+ progenitor cell,” which is a heterogeneous cell population that includes a subpopulation of HSCs, pluripotent stem cells and cells in the early stages of lineage commitment. CD34+ progenitor cells continuously migrate to and from the bone marrow in normal adult animals. They can differentiate to produce all hematopoietic cell lineages found in the circulation. In some embodiments, the hematopoietic cell is a very early progenitor CD34+ cell which is a subgroup of CD34+ progenitor cells enriched from HSCs.

In some embodiments, the hematopoietic cell is a “peripheral blood CD34+ cell”, which is a CD34+ cell present in the blood.

In some embodiments, the hematopoietic cell is a B cell CD19+ progenitor, which is a population of B-lineage cells that express cell surface CD10, CD34, and CD19.

In some embodiments, the hematopoietic cell is a myeloid progenitor CD13+ cells, which is a population of myeloid lineage cells that express cell surface CD34 and CD13, and in some cases, also CD33.

In some embodiments, the target cell is selected from the group consisting of myeloid-lymphoid balanced hematopoietic lineage cells, myeloid-biased hematopoietic lineage cells, lymphoid-biased hematopoietic lineage cells, a platelet-biased hematopoietic lineage cells, a platelet-myeloid-biased hematopoietic lineage cells, a long-term repopulating hematopoietic lineage cells, an intermediate-term repopulating hematopoietic lineage cells, or a short-term repopulating hematopoietic lineage cells. In some embodiments, the target cell is selected from monocytes, macrophages, neutrophils, basophils, eosinophils, erythrocytes, megakaryocytes and platelets. In some embodiments, the target cell is selected from T cells, B cells, natural killer (NK) cells and innate lymphoid cells.

In some embodiments the target cell is an effector cell, e.g., a cell of the immune system that expresses one or more Fc receptors and mediates one or more effector functions. In some embodiments, a target cell may include one or more of a monocyte, macrophage, neutrophil, dendritic cell, eosinophil, mast cell, platelet, large granular lymphocyte, Langerhans' cell, natural killer (NK) cell, T lymphocyte (e.g., T cell), a Gamma delta T cell, B lymphocyte (e.g., B cell) and may be from any organism including humans, mice, rats, rabbits, and monkeys.

In some embodiment, the hematopoietic cell is a T cell. In some embodiments, the T cell is a naïve T cell. In some embodiments, the T cell is a memory T cell.

In some embodiments, the hematopoietic cell is a B cell. In some embodiments, the target cell is a resting B cell, such as a naive or a memory B cell. In some embodiments, the target cell is a cancer B cell, such as a B-cell chronic lymphocytic leukemia (BCLL) cell or a marginal zone lymphoma (MZL) B cell.

In some embodiments, the target cell is a thymocyte. In some embodiments, the target cell is a natural killer (NK) cell. In some embodiments, the thymocyte expresses CD4 or CD8. In some embodiments, the thymocyte does not express CD4 or CD8. In some embodiments, the natural killer (NK) cell is a cell that expresses CD56.

In some embodiments, the target cell is a CD3+ T cell, a CD4+ T cell, or a CD8+ T cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, or a B cell.

In some embodiments, the binding domain (e.g. sdAb) variable domain binds a cell surface molecule or antigen. In some embodiments, the cell surface molecule is ASGR1, ASGR2, TM4SF5, CD3, CD8, CD4, CD7, or low density lipoprotein receptor (LDL-R). In some embodiments, the cell surface molecule is ASGR1. In some embodiments, the cell surface molecule is ASGR2. In some embodiments, the cell surface molecule is TM4SF5. In some embodiments, the cell surface molecule is CD3. In some embodiments, the cell surface molecule is CD8. In some embodiments, the cell surface molecule is CD4. In some embodiments, the cell surface molecule is LDL-R. In some embodiments, the cell surface molecule is ASCT2, CD105, CD110, CD117, CD133, CD146, CD164, CD34, CD46, CD49f, CD90, EPCR, or ITGA3.

In some embodiments, the retargeted attachment protein comprises the paramyxovirus envelope attachment protein (e.g., G protein or functionally active variant or biologically active portion thereof) linked directly to the binding domain and/or variable domain thereof. In some embodiments, the targeted envelope protein is a fusion protein that has the following structure: (N′-single domain antibody-C′)-(C′-G protein-N′).

In some embodiments, the retargeted attachment protein comprises the paramyxovirus envelope attachment protein (e.g., G protein or functionally active variant or biologically active portion thereof) linked indirectly via a linker to the binding domain and/or variable domain thereof. In some embodiments, the linker is a peptide linker. In some embodiments, the linker is a chemical linker.

In some embodiments, the linker is a peptide linker and the targeted envelope protein is a fusion protein containing the paramyxovirus envelope attachment protein (e.g., G protein or functionally active variant or biologically active portion thereof) linked via a peptide linker to the a binding molecule variable domain (e.g. antibody or antigen-binding fragment, e.g. sdAb or scFv variable domain). In some embodiments, the targeted envelope protein is a fusion protein that has the following structure: (N′-single domain antibody-C′)-Linker-(C′-G protein-N′).

In some embodiments, the peptide linker is up to 65 amino acids in length. In some embodiments, the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids. In some embodiments, the peptide linker is a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids in length.

In particular embodiments, the linker is a flexible peptide linker. In some such embodiments, the linker is 1-20 amino acids, such as 1-20 amino acids predominantly composed of glycine. In some embodiments, the linker is 1-20 amino acids, such as 1-20 amino acids predominantly composed of glycine and serine. In some embodiments, the linker is a flexible peptide linker containing amino acids Glycine and Serine, referred to as GS-linkers. In some embodiments, the peptide linker includes the sequences GS, GGS, GGGGS, GGGGGS or combinations thereof. In some embodiments, the polypeptide linker has the sequence (GGS)n, wherein n is 1 to 10. In some embodiments, the polypeptide linker has the sequence (GGGGS)n, wherein n is 1 to 10. In some embodiments, the polypeptide linker has the sequence (GGGGGS)n, wherein n is 1 to 6. In some embodiments, the retargeted attachment protein comprising a binding domain, a first binding domain, and/or a second binding domain linked to at least one paramyxovirus envelope attachment may comprise an engineered binding domain, such as an artificially generated binding domain. The binding domain may comprise a nanobody, a DARPin, an Aptamer, an Affimer, an Affibody, a Knottin, an Avimer, a Monobody, an Anticalin, a Fynomer. Any engineered binding domain known in art and suitable for the present invention can be used, for example any such binding domain described in Olaleye et al. Biomolecules. 2021 December; 11 (12): 1791.

### C. Binding Proteins

Exemplary CD8 binding agents include antibodies and fragments thereof (e.g., scFv, VHH) that bind to one or more of CD8 alpha and CD8 beta. Such antibodies may be derived from any species, and may be for example, mouse, rabbit, human, humanized, or camelid antibodies. Exemplary antibodies include those disclosed in WO2014025828, WO2014164553, WO2020069433, WO2015184203, US20160176969, WO2017134306, WO2019032661, WO2020257412, WO2018170096, WO2020060924, U.S. Pat. No. 10,730,944, US20200172620, and the non-human antibodies OKT8; RPA-T8, 12.C7 (Novus); 17D8, 3B5, LT8, RIV11, SP16, YTC182.20, MEM-31, MEM-87, RAVB3, C8/144B (Thermo Fisher); 2ST8.5H7, Bu88, 3C39, Hit8a, SPM548, CA-8, SK1, RPA-T8 (GeneTex); UCHT4 (Absolute Antibody); BW135/80 (Miltenyi); G42-8 (BD Biosciences); C8/1779R, mAB 104 (Enzo Life Sciences); B-Z31 (Sapphire North America); 32-M4, 5F10, MCD8, UCH-T4, 5F2 (Santa Cruz); D8A8Y, RPA-T8 (Cell Signaling Technology). Further exemplary anti-CD8 binding agents and G proteins are described in U.S. provisional application No. 63/172,518, which is incorporated by reference herein. Other exemplary binding agents include designed ankyrin repeat proteins (DARPins) and binding agents based on fibronectin type III (Fn3) scaffolds.

Non-limiting examples of antigen binding domains or antibodies or fragments thereof that bind to a CD8 antigen include those described in U.S. patent application Ser. No. 17/572,611 and U.S. Pat. No. 11,535,869, each of which is hereby incorporated by reference in its entirety.

In some embodiments, protein fusogens (e.g., attachment proteins) may be re-targeted by covalently conjugating a CD8 binding agent to the fusion protein or attachment protein (e.g. retargeted attachment protein). In some embodiments, the fusogen and CD8 binding agent are covalently conjugated by expression of a chimeric protein comprising the fusogen linked to the CD8 binding agent. In some embodiments, a single-chain variable fragment (scFv) can be conjugated to fusogens to redirect fusion activity towards cells that display the scFv binding target (doi: 10.1038/nbt1060, DOI 10.1182/blood-2012-11-468579, doi: 10.1038/nmeth.1514, doi: 10.1006/mthe.2002.0550, HUMAN GENE THERAPY 11:817-826, doi: 10.1038/nbt942, doi: 10.1371/journal.pone.0026381, DOI 10.1186/s12896-015-0142-z).

In some embodiments, designed ankyrin repeat proteins (DARPin) can be conjugated to fusogens to redirect fusion activity towards cells that display the DARPin binding target (doi: 10.1038/mt.2013.16, doi: 10.1038/mt.2010.298, doi: 10.4049/jimmunol.1500956), as well as combinations of different DARPins (doi: 10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′) 2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD8 binding agent to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi: 10.1038/nm1192). In some embodiments, altered and non-altered fusogens may be displayed on the same retroviral vector or VLP (doi: 10.1016/j.biomaterials.2014.01.051).

In some embodiments, a CD8 binding agent comprises a humanized antibody molecule, intact IgA, IgG, IgE or IgM antibody; bi- or multi-specific antibody (e.g., Zybodies®, etc.); antibody fragments such as Fab fragments, Fab′ fragments, F(ab′) 2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies® TrimerX®; MicroProteins; Fynomers®, Centyrins®; and KALBITOR®s.

In some embodiments, the CD8 binding agent is a peptide. In some embodiments, the CD8 binding agent is an antibody, such as a single-chain variable fragment (scFv). In some embodiments, the CD8 binding agent is an antibody, such as a single domain antibody. In some embodiments, the CD8 binding agent is a VHH. In some embodiments, the antibody can be human or humanized. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD8 binding agent is attached to the C-terminus of the G protein (e.g., fusogen) or biologically active portion thereof. In some embodiments, the N-terminus of the CD8 binding agent is exposed on the exterior surface of the lipid bilayer.

In some embodiments, the CD8 binding agent is the only surface displayed non-viral sequence of the viral vector. In some embodiments, the CD8 binding agent is the only membrane bound non-viral sequence of the viral vector. In some embodiments, the viral vector does not contain a molecule that engages or stimulates T cells other than the CD8 binding agent.

In some embodiments, viral vectors may display CD8 binding agents that are not conjugated to protein fusogens in order to redirect the fusion activity towards a cell that is bound by the targeting moiety, or to affect homing

The lentiviral particles disclosed herein include, in some embodiments, one or more CD4 binding agents. For example, a CD4 binding agent may be fused to or incorporated in a protein fusogen or attachment protein. In another embodiment, a CD4 binding agent may be incorporated into the viral particle envelope via fusion with a transmembrane domain.

In some of any of the provided embodiments, the CD4 binding agent is exposed on the surface of the viral particle. In some embodiments, the CD4 binding agent is fused to a transmembrane domain incorporated in the viral particle envelope.

Non-limiting examples of antigen binding domains or antibodies or fragments thereof that bind to a CD4 antigen include those described in U.S. patent application Ser. No. 18/294,520, which is hereby incorporated by reference in its entirety.

Exemplary CD4 binding agents include antibodies and fragments thereof (e.g., scFv, VHH) that bind to CD4. Such antibodies may be derived from any species, and may be for example, mouse, rabbit, human, humanized, or camelid antibodies. Exemplary antibodies include ibalizumab, zanolimumab, tregalizumab, priliximab, cedelizumab, clenoliximab, keliximab, and anti-CD4 antibodies disclosed in WO2002102853, WO2004083247, WO2004067554, WO2007109052, WO2008134046, WO2010074266, WO2012113348, WO2013188870, WO2017104735, WO2018035001, WO2018170096, WO2019203497, WO2019236684, WO2020228824, U.S. Pat. Nos. 5,871,732, 7,338,658, 7,722,873, 8,399,621, 8,911,728, 9,005,963,U.S. Pat. Nos. 9,587,022, 9,745,552, U.S. provisional application No. 63/326,269, U.S. provisional application No. 63/341,681; as well as antibodies B486A1, RPA-T4, CE9.1 (Novus Biologicals); GK1.5, RM4-5, RPA-T4, OKT4, 4SM95, S3.5, N1UGO (ThermoFisher); GTX50984, ST0488, 10B5, EP204 (GeneTex); GK1.3, 5A8, 10C12, W3/25, 8A5, 13B8.2, 6G5 (Absolute Antibody); VIT4, M-T466, M-T321, REA623, (Miltenyi); MEM115, MT310 (Enzo Life Sciences); H129.19, 5B4, 6A17, 18-46, A-1, C-1, OX68 (Santa Cruz); EP204, D2E6M (Cell Signaling Technology). Other exemplary binding agents include designed ankyrin repeat proteins (DARPins) (e.g., the anti-CD4 DARPin disclosed in WO2017182585) and binding agents based on fibronectin type III (Fn3) scaffolds. Each of U.S. Pat. No. 9,005,963, U.S. provisional application No. 63/326,269, and U.S. provisional application No. 63/341,681 is incorporated by reference herein in its entirety.

Exemplary CD3 binding agents include antibodies and fragments thereof (e.g., scFv, VHH) that bind to CD3. Such antibodies may be derived from any species, and may be for example, mouse, rabbit, human, humanized, or camelid antibodies.

Non-limiting examples of antigen binding domains or antibodies or fragments thereof that bind to a CD3 antigen include those described in International Patent Publication No. WO 2023/150518.

Exemplary antibodies include OKT3, CRIS-7, I2C, blinatumomab, catumaxomab, muromonab-CD3, A-319, AFM11, AMG 199, AMG 211, AMG 424, AMG 427, AMG 562, AMG 564, APVO436, CC-93269, ERY974, GBR1302, GEM333, GEM2PSCA, GNC-035, HPN424, IGM-2323, JNJ-63709178, JNJ-63898081, JNJ-75348780, JNJ-78306358, M701, M802, MGD007, MOR209/ES414, PF-06671008, REGN5459, RO7283420, SAR442257, SAR443216, TNB-383B, TNB-486, TNB-585, Y150, acapatamab, cevostamab, cibisatamab, duvortuxizumab, eluvixtamab, emerfetamab, etevritamab, glofitamab, gresonitamab, obrindatamab, pavurutamab, plamotamab, solitomab, tarlatamab, tepoditamab, tidutamab, vibecotamab, vixtimotamab, alnuctamab, dafsolimab setaritox, pacanalotamab, pasotuxizumab, runimotamab, nivatrotamab, elranatamab, ertumaxomab, flotetuzumab, odronextamab, talquetamab, teclistamab, visilizumab, epcoritamab, otelixizumab, 3F8BiAb, CCW702, DKTK CC-1, EMB-06, GEN1044, GEN1047, GTB-3550, HPN217, IMC-C103C, NVG-111, REGN4018, REGN4336, REGN5458, A-2019, A-337, ABP-100, AFM15, AFM21, AMG 701, APVO425, CLN-049, Dow2, EM801, Ektomab, FBTA05, GBR1342, GBR1372, GSK3537142, HBM7020, HLX31, IGM-2644, MG1122, MGD015, ND003, ND007, PF-07062119, RO7293583, STA551, TT19, ZW38; and anti-CD3 antibodies disclosed in U.S. Pat. Nos. 4,361,549, 7,728,114, 9,657,102, 9,587,021, and 11007267; US Patent Application Nos. US20120269826, US20180057597, and US20180112000; and PCT Application Nos. WO2005118635, WO2011050106, WO2012162067, WO2014047231, WO2016116626, WO2016180721, and WO2016204966. Other exemplary binding agents include designed ankyrin repeat proteins (DARPins) and binding agents based on fibronectin type III (Fn3) scaffolds.

In some embodiments, the viral particles disclosed herein comprise one or more retargeted attachment proteins, each independently comprising (i) a paramyxovirus envelope attachment protein; and (ii) a targeting moiety directed to a target molecule expressed on the surface of a target cell. In some embodiments, the targeting moiety is an HSC binding domain, e.g., an HSC binding agent, such as any of those disclosed herein.

The viral particles disclosed herein include, in some embodiments, one or more HSC binding domains (e.g., HSC binding agent) that target the viral vector to a cell that is an HSC. In some embodiments, the HSC binding agent binds to a molecule expressed on the surface of the HSC. The cell surface molecule may be a receptor, coreceptor, or a GPI-anchored protein. In some embodiments, the HSC binding agent binds ASCT2, CD105, CD110, CD117, CD133, CD146, CD164, CD34, CD46, CD49f, CD90, EPCR, or ITGA3. In some embodiments, a HSC binding agent may be fused to or incorporated in a protein fusogen or viral particle envelope attachment protein (e.g., a retargeted attachment protein). In some embodiments, a HSC binding agent may be incorporated into the viral envelope via fusion with a transmembrane domain. In some embodiments, the HSC binding agent targets the viral particle to a HSC.

In particular embodiments, a HSC binding agent may be fused to or incorporated in a protein fusogen or attachment protein, thereby retargeting the viral particle to a HSC. In some embodiments, for re-targeting the HSC binding agent is fused to a protein fusogen or envelope attachment protein that is mutated to reduce binding for the native binding partner of the fusogen or viral envelope protein. In some embodiments, the fusogen is or contains a mutant G protein or a biologically active portion thereof that is a mutant of wild-type NiV-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi: 10.1038/nbt942, Molecular Therapy vol. 16 no. 8, 1427-1436 August 2008, doi: 10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a HSC binding agent to the attachment protein. In some embodiments, the fusogen and HSC binding agent are covalently conjugated by expression of a chimeric protein comprising the fusogen linked to the HSC binding agent (e.g., retargeted attachment protein). The HSC binding agent can include any targeting protein able to confer specific binding to a target molecule expressed on the surface of a HSC. In some embodiments, a targeting protein can also include an antibody or an antigen-binding fragment thereof (e.g., Fab, Fab′, F(ab′) 2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, the HSC binding agent is an antibody or antigen binding fragment thereof. In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi: 10.1038/nm1192). In some embodiments, altered and non-altered fusogens may be displayed on the same retroviral vector or VLP (doi: 10.1016/j.biomaterials.2014.01.051).

In some embodiments, a single-chain variable fragment (scFv) can be conjugated to fusogens to redirect fusion activity towards HSCs that display the scFv binding target (doi: 10.1038/nbt1060, DOI 10.1182/blood-2012-11-468579, doi: 10.1038/nmeth.1514, doi: 10.1006/mthe.2002.0550, HUMAN GENE THERAPY 11:817-826, doi: 10.1038/nbt942, doi: 10.1371/journal.pone.0026381, DOI 10.1186/s12896-015-0142-z). In some embodiments, designed ankyrin repeat proteins (DARPin) can be conjugated to fusogens to redirect fusion activity towards HSCs that display the DARPin binding target (doi: 10.1038/mt.2013.16, doi: 10.1038/mt.2010.298, doi: 10.4049/jimmunol.1500956), as well as combinations of different DARPins (doi: 10.1038/mto.2016.3). In some embodiments, a single domain antibody (e.g., a VHH) can be conjugated to fusogens to redirect fusion activity towards HSCs that display the sdAb binding target. In some embodiments, receptor ligands and antigens can be conjugated to fusogens to redirect fusion activity towards HSCs that display the target receptor (DOI: 10.1089/hgtb.2012.054, DOI: 10.1128/JVI.76.7.3558-3563.2002).

In some embodiments, the target cell is a CD34+ progenitor cells. In some embodiments, the target cell molecule is expressed on at least a subset of CD34+ progenitor cells.

In some embodiments, the cell surface molecule is expressed on HSCs. In some embodiments, the cell surface molecule is expressed on MPPs. In some embodiments, the cell surface molecule is expressed on MLPs. In some embodiments, the cell surface molecule is expressed on ETPs. In some embodiments, the cell surface molecule is expressed on MEPs. In some embodiments, the cell surface molecule is expressed on CMPs. In some embodiments, the cell surface molecule is expressed on GMPs. In some embodiments, the cell surface molecule is expressed on any combination of the foregoing CD34+ progenitor subpopulations. In some embodiments, the cell surface molecule is expressed on HSCs and MPPs. In some embodiments, the cell surface molecule is expressed on myeloid progenitors. In some embodiments, the cell surface molecule is expressed on lymphoid progenitors. In some embodiments, the cell surface molecule is expressed on myeloid progenitors. In some embodiments, the cell surface molecule is expressed on HSCs, MPPs, MEPs, CMPs, and GMPs.

In some embodiments, the cell surface molecule is ASCT2. In some embodiments, the target cell is ASCT2+.

In some embodiments, the cell surface molecule is CD105. In some embodiments, the target cell is CD105+.

In some embodiments, the cell surface molecule is CD110. In some embodiments, the target cell is CD110+.

In some embodiments, the cell surface molecule is CD117. In some embodiments, the target cell is CD117+.

In some embodiments, the cell surface molecule is CD133. In some embodiments, the target cell is CD133+.

In some embodiments, the cell surface molecule is CD146. In some embodiments, the target cell is CD146+.

In some embodiments, the cell surface molecule is CD164. In some embodiments, the target cell is CD164+.

In some embodiments, the cell surface molecule is CD34. In some embodiments, the target cell is CD34+.

In some embodiments, the cell surface molecule is CD46. In some embodiments, the target cell is CD46+.

In some embodiments, the cell surface molecule is CD49f. In some embodiments, the target cell is CD49f+.

In some embodiments, the ta cell surface molecule is CD90. In some embodiments, the target cell is CD90+.

In some embodiments, the cell surface molecule is EPCR. In some embodiments, the target cell is EPCR+.

In some embodiments, the cell surface molecule is ITGA3. In some embodiments, the target cell is ITGA3+.

In some embodiments, the target molecule is CD133. In some embodiments, the target cell is CD133+. In some embodiments, the targeting agent is an anti-CD133 antibody. Exemplary anti-CD133 antibodies include CART133, AC133, 293C3-SDIE, CMab-43, RW03, 293C3H9 (293C3), and W6B3H10 (W6B3); and anti-CD133 antibodies disclosed in US Patent Nos. U.S. Pat. Nos. 8,722,858, 9,249,225, 9,624,303, 10,106,623, 10,711,068, 11,098,109, 11,214,628, 11,352,435, and 11,220,551; US Patent Application Nos. US20130224202; PCT Application Nos. WO200901840, WO2011089211, WO2011149493, WO2014128185, WO2015121383, WO2016154623, WO2018045880, WO2018072025, and WO2022022718; and Canadian Patent Application No. CA2962157.

Non-limiting examples of antigen binding domains or antibodies or fragments thereof that bind to a CD133 antigen include those described in International Patent App. No. PCT/US2023/076747, which is hereby incorporated by reference in its entirety.

In some embodiments, the viral particles disclosed herein comprise one or more retargeted attachment proteins, each independently comprising (i) a paramyxovirus envelope attachment protein; and (ii) a targeting moiety directed to a target molecule expressed on the surface of a target cell, wherein the target molecule is CD133. In some embodiments, the targeting moiety is a CD133 binding domain, e.g., a CD133 binding agent, such as any of those disclosed herein.

In some embodiments, the viral particles comprise one or more HSC binding domains that is a CD133 binding agent that targets the viral vector to a cell that is an HSC. In some embodiments, the viral particles comprise two or more HSC binding domains that are each a CD133 binding agent that targets the viral vector to a cell that is an HSC. In some embodiments, each of the two or more HSC binding domains that are each a CD133 binding agent bind distinct epitopes of the same target molecule (CD133). In some embodiments, the viral particle comprises two or more, e.g., two, three, four, or five or more, CD133 binding agents.

Non-limiting examples of antigen binding domains or antibodies or fragments thereof that bind to a CD117 antigen include those described in International Patent App. No. PCT/US2023/076747, which is hereby incorporated by reference in its entirety.

D. F proteins

In some embodiments, the viral particle comprises one or more paramyxovirus fusion (F) proteins. In some embodiments, the viral particle contains an exogenous or overexpressed paramyxovirus fusion (F) protein. In some embodiments, the paramyxovirus fusion (F) protein is disposed in the lipid bilayer. In some embodiments, the paramyxovirus fusion (F) protein (e.g., fusogen) facilitates the fusion of the viral particle to a membrane. In some embodiments, the membrane is a plasma cell membrane. In some embodiments, the paramyxovirus fusion (F) protein binds a binding partner on a target cell surface. In some embodiments, the paramyxovirus fusion (F) protein comprises a protein with a hydrophobic fusion peptide domain.

In some embodiments the paramyxovirus fusion (F) protein is or comprises a Nipah virus protein F, a measles virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein.

In some embodiments, the paramyxovirus fusion (F) protein comprises a henipavirus F protein molecule or biologically active portion thereof. In some embodiments, the Henipavirus F protein is a Hendra (HeV) virus F protein, a Nipah (NiV) virus F-protein, a Cedar (CedPV) virus F protein, a Mòjiang virus F protein, a Langya virus F protein or a bat Paramyxovirus F protein or a biologically active portion thereof.

Table 2 provides non-limiting examples of F proteins. In some embodiments, the N-terminal hydrophobic fusion peptide domain of the F protein molecule or biologically active portion thereof is exposed on the outside of lipid bilayer.

In some embodiments, the paramyxovirus fusion (F) protein is a variant Nipah F protein (NiV-F). In some embodiments, the variant NiV-F protein exhibits fusogenic activity. In some embodiments, the variant NiV-F facilitates the fusion of the viral particle (e.g. lentiviral vector) to a membrane. F proteins of henipaviruses, including NiV-F, are encoded as FO precursors containing a signal peptide (e.g. corresponding to amino acid residues 1-26 of the below). Following cleavage of the signal peptide, the mature FO (SEQ ID NO:14 lacking the signal peptide, i.e. set forth in SEQ ID NO:15) is transported to the cell surface, then endocytosed and cleaved by cathepsin L (e.g. between amino acids 109-110 of NiV-F corresponding to amino acids set forth in SEQ ID NO:16) into the mature fusogenic subunits F1 (e.g. corresponding to amino acids 110-546 of NiV-F set forth in SEQ ID NO:16) and F2 (e.g. corresponding to amino acid residues 27-109 of NiV-F set forth in SEQ ID NO:16). The F1 and F2 subunits are associated by a disulfide bond and recycled back to the cell surface. The F1 subunit contains the fusion peptide domain located at the N terminus of the F1 subunit (e.g. corresponding to amino acids 110-129 of the below e.g. NiV-F set forth in SEQ ID NO:16) where it is able to insert into a cell membrane to drive fusion. In particular cases, fusion activity is blocked by association of the F protein with G protein, until G engages with a target molecule resulting in its disassociation from F and exposure of the fusion peptide to mediate membrane fusion.

Among different henipavirus species, the sequence and activity of the F protein is highly conserved. For examples, the F protein of NiV and HeV viruses share 89% amino acid sequence identity. Further, in some cases, the henipavirus F proteins exhibit compatibility with G proteins from other species to trigger fusion (Brandel-Tretheway et al. Journal of Virology. 2019. 93(13):e00577-19). In some aspects of the provided viral particles, the F protein is heterologous to the G protein, i.e. the F and G protein or biologically active portions are from different henipavirus species. For example, the F protein is from Hendra virus and the G protein is from Nipah virus. In other aspects, the F protein can be a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. 2019). In some cases, the chimeric F protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus. F protein sequences disclosed herein are predominantly disclosed as expressed sequences including an N-terminal signal sequence. As such N-terminal signal sequences are commonly cleaved co- or post-translationally, the mature protein sequences for all F protein sequences disclosed herein are also contemplated as lacking the N-terminal signal sequence.

TABLE 2
Table 2: Non-limiting Examples of F Proteins

| | | | | | SEQ ID |
|---|---|---|---|---|---|
| | Nucleo- | | | | NO |
| Gen | tides | Full | | | (without |
| bank | of | Gene | | SEQ | signal |
| ID | CDS | Name | Sequence | ID | sequence) |
| AF017149 | 6618- | gb: | MATQEVRLKCLLCGIIVLVLSLEGLGILHYEKLSKIGLVKGITRK | 17 | 14 |
| | 8258 | AF017149\| | YKIKSNPLTKDIVIKMIPNVSNVSKCTGTVMENYKSRLTGILSPI | | |
| | | Organism: | KGAIELYNNNTHDLVGDVKLAGVVMAGIAIGIATAAQITAGVALY | | |
| | | Hendravirus\| | EAMKNADNINKLKSSIESTNEAVVKLQETAEKTVYVLTALQDYIN | | |
| | | Strain | TNLVPTIDQISCKQTELALDLALSKYLSDLLFVFGPNLQDPVSNS | | |
| | | Name: | MTIQAISQAFGGNYETLLRTLGYATEDFDDLLESDSIAGQIVYVD | | |
| | | UNKNOWN- | LSSYYIIVRVYFPILTEIQQAYVQELLPVSFNNDNSEWISIVPNF | | |
| | | AF017149\| | VLIRNTLISNIEVKYCLITKKSVICNQDYATPMTASVRECLTGST | | |
| | | Protein | DKCPRELVVSSHVPRFALSGGVLFANCISVTCQCQTTGRAISQSG | | |
| | | Name: | EQTLLMIDNTTCTTVVLGNIIISLGKYLGSINYNSESIAVGPPVY | | |
| | | fusion\| | TDKVDISSQISSMNQSLQQSKDYIKEAQKILDTVNPSLISMLSMI | | |
| | | Gene | ILYVLSIAALCIGLITFISFVIVEKKRGNYSRLDDRQVRPVSNGD | | |
| | | Symbol: | LYYIGT | | |
| | | F | | | |
| Q9IH63 | | Additional | MVVILDKRCYCNLLILILMISECSVGILHYEKLSKIGLVKGVTRK | 16 | 15 |
| | | incluster: | YKIKSNPLTKDIVIKMIPNVSNMSQCTGSVMENYKTRLNGILTPI | | |
| | | sp Q91H63\| | KGALEIYKNNTHDLVGDVRLAGVIMAGVAIGIATAAQITAGVALY | | |
| | | FUS_NIPAV | EAMKNADNINKLKSSIESTNEAVVKLQETAEKTVYVLTALQDYIN | | |
| | | Fusion | TNLVPTIDKISCKQTELSLDLALSKYLSDLLFVFGPNLQDPVSNS | | |
| | | glycoprotein | MTIQAISQAFGGNYETLLRTLGYATEDFDDLLESDSITGQIIYVD | | |
| | | F0OS = | LSSYYIIVRVYFPILTEIQQAYIQELLPVSFNNDNSEWISIVPNF | | |
| | | Nipah virus | ILVRNTLISNIEIGFCLITKRSVICNQDYATPMTNNMRECLTGST | | |
| | | | EKCPRELVVSSHVPRFALSNGVLFANCISVTCQCQTTGRAISQSG | | |
| | | | EQTLLMIDNTTCPTAVLGNVIISLGKYLGSVNYNSEGIAIGPPVF | | |
| | | | TDKVDISSQISSMNQSLQQSKDYIKEAQRLLDTVNPSLISMLSMI | | |
| | | | ILYVLSIASLCIGLITFISFIIVEKKRNTYSRLEDRRVRPTSSGD | | |
| | | | LYYIGT | | |
| JQ001776 | 6129- | gb: | MSNKRTTVLIIISYTLFYLNNAAIVGFDFDKLNKIGVVQGRVLNY | 18 | 19 |
| | 8166 | JQ001776: | KIKGDPMTKDLVLKFIPNIVNITECVREPLSRYNETVRRLLLPIH | | |
| | | 6129- | NMLGLYLNNTNAKMTGLMIAGVIMGGIAIGIATAAQITAGFALYE | | |
| | | 8166\| | AKKNTENIQKLTDSIMKTQDSIDKLTDSVGTSILILNKLQTYINN | | |
| | | Organism: | QLVPNLELLSCRQNKIEFDLMLTKYLVDLMTVIGPNINNPVNKDM | | |
| | | Cedarvirus\| | TIQSLSLLFDGNYDIMMSELGYTPQDFLDLIESKSITGQIIYVDM | | |
| | | Strain | ENLYVVIRTYLPTLIEVPDAQIYEFNKITMSSNGGEYLSTIPNFI | | |
| | | Name: | LIRGNYMSNIDVATCYMTKASVICNQDYSLPMSQNLRSCYQGETE | | |
| | | CGla\| | YCPVEAVIASHSPRFALTNGVIFANCINTICRCQDNGKTITQNIN | | |
| | | Protein | QFVSMIDNSTCNDVMVDKFTIKVGKYMGRKDINNINIQIGPQIII | | |
| | | Name: | DKVDLSNEINKMNQSLKDSIFYLREAKRILDSVNISLISPSVQLF | | |
| | | Fusion | LIIISVLSFIILLIIIVYLYCKSKHSYKYNKFIDDPDYYNDYKRE | | |
| | | glycoprotein\| | RINGKASKSNNIYYVGD | | |
| | | Gene | | | |
| | | Symbol: | | | |
| | | F | | | |
| NC_025352 | 5950- | gb: | MALNKNMFSSLFLGYLLVYATTVQSSIHYDSLSKVGVIKGLTYNY | 20 | 21 |
| | 8712 | NC_025352: | KIKGSPSTKLMVVKLIPNIDSVKNCTQKQYDEYKNLVRKALEPVK | | |
| | | 5950- | MAIDTMLNNVKSGNNKYRFAGAIMAGVALGVATAATVTAGIALHR | | |
| | | 8712\| | SNENAQAIANMKSAIQNTNEAVKQLQLANKQTLAVIDTIRGEINN | | |
| | | Organism: | NIIPVINQLSCDTIGLSVGIRLTQYYSEIITAFGPALQNPVNTRI | | |
| | | Mojiangvirus\| | TIQAISSVENGNFDELLKIMGYTSGDLYEILHSELIRGNIIDVDV | | |
| | | Strain | DAGYIALEIEFPNLTLVPNAVVQELMPISYNIDGDEWVTLVPRFV | | |
| | | Name: | LTRTTLLSNIDTSRCTITDSSVICDNDYALPMSHELIGCLQGDTS | | |
| | | Tongguan1\| | KCAREKVVSSYVPKFALSDGLVYANCLNTICRCMDTDTPISQSLG | | |
| | | Protein | ATVSLLDNKRCSVYQVGDVLISVGSYLGDGEYNADNVELGPPIVI | | |
| | | Name: | DKIDIGNQLAGINQTLQEAEDYIEKSEEFLKGVNPSIITLGSMVV | | |
| | | Fusion | LYIFMILIAIVSVIALVLSIKLTVKGNVVRQQFTYTQHVPSMENI | | |
| | | protein\| | NYVSH | | |
| | | Gene | | | |
| | | Symbol: | | | |
| | | F | | | |
| NC_025256 | 6865- | gb: | MKKKTDNPTISKRGHNHSRGIKSRALLRETDNYSNGLIVENLVRN | 22 | 23 |
| | 8853 | NC_025256: | CHHPSKNNLNYTKTQKRDSTIPYRVEERKGHYPKIKHLIDKSYKH | | |
| | | 6865- | IKRGKRRNGHNGNIITIILLLILILKTQMSEGAIHYETLSKIGLI | | |
| | | 8853\| | KGITREYKVKGTPSSKDIVIKLIPNVTGLNKCTNISMENYKEQLD | | |
| | | Organism: | KILIPINNIIELYANSTKSAPGNARFAGVIIAGVALGVAAAAQIT | | |
| | | Bat | AGIALHEARQNAERINLLKDSISATNNAVAELQEATGGIVNVITGM | | |
| | | Paramyxovirus | QDYINTNLVPQIDKLQCSQIKTALDISLSQYYSEILTVFGPNLQN | | |
| | | Eid_hel/GH- | PVTTSMSIQAISQSFGGNIDLLLNLLGYTANDLLDLLESKSITGQ | | |
| | | M74a/GHA/ | ITYINLEHYFMVIRVYYPIMTTISNAYVQELIKISFNVDGSEWVS | | |
| | | 2009\| | LVPSYILIRNSYLSNIDISECLITKNSVICRHDFAMPMSYTLKEC | | |
| | | Strain | LTGDTEKCPREAVVTSYVPRFAISGGVIYANCLSTTCQCYQTGKV | | |
| | | Name: | IAQDGSQTLMMIDNQTCSIVRIEEILISTGKYLGSQEYNTMHVSV | | |
| | | BatPV/ | GNPVFTDKLDITSQISNINQSIEQSKFYLDKSKAILDKINLNLIG | | |
| | | Eid_hel/ | SVPISILFIIAILSLILSIITFVIVMIIVRRYNKYTPLINSDPSS | | |
| | | GH-M74a/ | RRSTIQDVYIIPNPGEHSIRSAARSIDRDRD | | |
| | | GHA/2009\| | | | |
| | | Protein | | | |
| | | Name: | | | |
| | | Fusion | | | |
| | | protein\| | | | |
| | | Gene | | | |
| | | Symbol: | | | |
| | | F | | | |

In some embodiments, the F protein or the biologically active portion thereof is a wild-type Nipah virus F (NiV-F) protein or a Hendra virus F protein or is a functionally active variant or biologically active portion thereof. For instance, in some embodiments, the F protein or the biologically active portion thereof is a wild-type NiV-F protein or a functionally active variant or a biologically active portion thereof.

In some embodiments, the F protein has the sequence of amino acids set forth in SEQ ID NO:17, SEQ ID NO:16, SEQ ID NO:18, SEQ ID NO:20, or SEQ ID NO:22, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:17, SEQ ID NO:16, SEQ ID NO:18, SEQ ID NO: 20, or SEQ ID NO:22, and retains fusogenic activity in conjunction with a G protein, such as a variant NiV-G as provided herein. In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:17, SEQ ID NO: 16, SEQ ID NO:18, SEQ ID NO:20, or SEQ ID NO:22.

In particular embodiments, the F protein has the sequence of amino acids set forth in SEQ ID NO:14, SEQ ID NO:15, SEQ ID NO:19, SEQ ID NO:21, or SEQ ID NO:23, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:14, SEQ ID NO:15, SEQ ID NO: 19, SEQ ID NO: 21, or SEQ ID NO:23, and retains fusogenic activity in conjunction with a G protein, such as a variant NiV-G as provided herein. In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:14, SEQ ID NO: 15, SEQ ID NO:19, SEQ ID NO:21, or SEQ ID NO:23.

Fusogenic activity includes the activity of the paramyxovirus fusion (F) protein in conjunction with a paramyxovirus envelope protein (e.g., G protein or G proteins) to promote or facilitate fusion of two membrane lumens, such as the lumen of the targeted viral particle having embedded in its lipid bilayer a henipavirus F and at least two G proteins, and a cytoplasm of a target cell, e.g. a cell that contains a surface receptor or molecule that is recognized or bound by the targeted envelope protein. In some embodiments, the F protein and at least one G protein are from the same Henipavirus species (e.g. NiV-G and NiV-F). In some embodiments, the F protein and at least one G protein are from different Henipavirus species (e.g. NiV-G and HeV-F). In particular embodiments, the F protein of the functionally active variant or biologically active portion retains the cleavage site cleaved by cathepsin L (e.g. corresponding to the cleavage site between amino acids 109-110 of SEQ ID NO:16).

Reference to retaining fusogenic activity includes activity (in conjunction with a G protein, such as a variant G protein provided herein) that is between at or about 10% and at or about 150% or more of the level or degree of binding of the corresponding wild-type F protein, such as set forth in SEQ ID NO:17, SEQ ID NO:16, SEQ ID NO:18, SEQ ID NO:20, or SEQ ID NO: 22, SEQ ID NO:14, SEQ ID NO: 15, SEQ ID NO: 19, SEQ ID NO:21, or SEQ ID NO:23 or a cathepsin L cleaved from thereof containing an F1 and F2 subunit. In some embodiments, the fusogenic activity is at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type F protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the paramyxovirus fusion (F) protein is a mutant F protein that is a functionally active fragment or a biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (Hev) virus F protein, a Nipah (NiV) virus F-protein, a Cedar (CedPV) virus F protein, a Mojiang virus F protein or a bat Paramyxovirus F protein. In some embodiments, the wild-type F protein is encoded by a sequence of nucleotides that encodes any one of SEQ ID NO:17, SEQ ID NO: 16, SEQ ID NO:18, SEQ ID NO:20, or SEQ ID NO:22, SEQ ID NO:14, SEQ ID NO:15, SEQ ID NO:19, SEQ ID NO:21, or SEQ ID NO:23 or a cathepsin L cleaved from thereof containing an F1 and F2 subunit.

In some embodiments, the mutant F protein is a biologically active portion that is truncated and lacks up to 22 contiguous amino acid residues at or near the C-terminus of the wild-type F protein, such as a wild-type F protein set forth in any one of SEQ ID NO:17, SEQ ID NO: 16, SEQ ID NO:18, SEQ ID NO:20, or SEQ ID NO:22, SEQ ID NO:14, SEQ ID NO: 15, SEQ ID NO:19, SEQ ID NO:21, or SEQ ID NO:23. In some embodiments, the mutant F protein is truncated and lacks up to 22 contiguous amino acids, such as up to 21, 20, 19, 18, 17, 16, 15, 14, 13, 12, 11, 10, 9, 8, 7, 6, 5, 4, 3, 2, or 1 contiguous amino acids at the C-terminus of the wild-type F protein.

In some embodiments, the NiV-F, such as a mutant or truncated NiV-F, of a provided viral particle includes the FO precursor or a proteolytically cleaved form thereof containing the F1 and F2 subunits, such as resulting following proteolytic cleavage at the cleavage site (e.g. between amino acids corresponding to amino acids between amino acids 109-110 of SEQ ID NO:16) to produce two chains that can be linked by disulfide bond. In some embodiments, the NiV-F, such as wild-type NiV-F or a truncated or mutated NiV-F protein, is produced or encoded as an FO precursor which then is able to be proteolytically cleaved to result in an F protein containing the F1 and F2 subunit linked by a disulfide bond. Hence, it is understood that reference to a particular sequence (SEQ ID NO) of a NiV-F herein is typically with reference to the FO precursor sequence but also is understood to include the proteolytically cleaved form or sequence thereof containing the two cleaved chains, F1 and F2. For instance, the NiV-F, such as a mutant or truncated NiV-F, contains an F1 subunit corresponding to amino acids 110-546 of NiV-F set forth in SEQ ID NO:16 or truncated or mutant sequence thereof, and an F2 corresponding to amino acid residues 27-109 of NiV-F set forth in SEQ ID NO:16.

In some embodiments, the mutant F protein is a biologically active portion that is truncated and lacks up to 22 contiguous amino acid residues at or near the C-terminus of the wild-type NiV-F protein, such as a wild-type NiV-F protein set forth in SEQ ID NO:16 or SEQ ID NO: 15. In some embodiments, the mutant F protein is truncated and lacks up to 22 contiguous amino acids, such as up to 21, 20, 19, 18, 17, 16, 15, 14, 13, 12, 11, 10, 9, 8, 7, 6, 5, 4, 3, 2, or 1 contiguous amino acids at the C-terminus of the wild-type NiV-F protein, such as a wild-type NiV-F protein set forth in SEQ ID NO:16 or SEQ ID NO:15. In some embodiments, the mutant F protein contains an F1 subunit and an F2 subunit in which (1) the Fl subunit is truncated and lacks up to 22 contiguous amino acids at or near the C-terminus of the wild-type F1 subunit, such as lacks up to 22 contiguous amino acids at or near the C-terminus of the wild-type F1 subunit corresponding to amino acids 110-546 of NiV-F set forth in SEQ ID NO:16, and (2) the F2 subunit has the sequence corresponding to amino acid residues 27-109 of NiV-F set forth in SEQ ID NO:15.

In some embodiments, the paramyxovirus fusion (F) protein is a mutant NiV-F protein that is a biologically active portion thereof that comprises a 22 amino acid truncation at or near the C-terminus of the wild-type NiV-F protein (SEQ ID NO:16 or SEQ ID NO:15). In some embodiments, the NiV-F protein is encoded by a nucleotide sequence that encodes the sequence set forth in SEQ ID NO: 226. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 24. In particular embodiments, the variant F protein is a mutant NiV-F protein that has the sequence of amino acids set forth in SEQ ID NO: 25. In some embodiments, the NiV-F proteins is encoded by a sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 25. In some embodiments, the F protein molecule or biologically active portion thereof comprises the sequence set forth in SEQ ID NO: 25.

In some embodiments, the mutant F protein contains an F1 subunit and an F2 subunit in which (1) the Fl subunit is set forth as amino acids 110-524 of SEQ ID NO:24, and (2) the F2 subunit is set forth as amino acids 27-109 of SEQ ID NO:24.

In some embodiments, the mutant F protein contains an F1 subunit and an F2 subunit in which (1) the Fl subunit is set forth as amino acids 84-498 of SEQ ID NO:25, and (2) the F2 subunit is set forth as amino acids 1-83 of SEQ ID NO:25.

### E. Polynucleotides

Provided herein are polynucleotides comprising a nucleic acid sequence encoding a retargeted attachment protein. Also provided herein are polynucleotides encoding at least two retargeted attachment proteins. In some embodiments, the polynucleotides comprise a nucleic acid sequence encoding a G protein, F protein, or biologically active portion thereof. In some embodiments, the polynucleotides comprise a nucleic acid sequence encoding a first G protein, and a second G protein, or biologically active portion thereof. In some embodiments, the polynucleotides comprise a nucleic acid sequence encoding a first G protein, a second G protein, an F protein, or biologically active portion thereof. In some embodiments, the polynucleotides comprise a nucleic acid sequence encoding a first G protein, a second G protein, a third G protein, an F protein, or biologically active portion thereof. In some embodiments, the polynucleotides comprise a nucleic acid sequence encoding a first G protein, a second G protein, a third G protein, a fourth G protein, an F protein, or biologically active portion thereof. In some embodiments, the polynucleotides comprise a nucleic acid sequence encoding a first G protein, a second G protein, a third G protein, a fourth G protein, a fifth G protein, an F protein, or biologically active portion thereof. In some embodiments, the polynucleotides comprise a nucleic acid sequence encoding a first G protein, a second G protein, a third G protein, a fourth G protein, a fifth G protein, one or more additional G proteins, an F protein, or biologically active portion thereof. In some embodiments, the polynucleotides further comprise a nucleic acid sequence encoding a binding domain, such as single domain antibody (sdAb) variable domain or biologically active portion thereof. The polynucleotides may include a sequence of nucleotides encoding any of the chimeric attachment described above. The polynucleotide can be a synthetic nucleic acid. Also provided are expression vector containing any of the provided polynucleotides.

In some of any embodiments, expression of natural or synthetic nucleic acids is typically achieved by operably linking a nucleic acid encoding the gene of interest to a promoter and incorporating the construct into an expression vector. In some embodiments, vectors can be suitable for replication and integration in eukaryotes. In some embodiments, cloning vectors contain transcription and translation terminators, initiation sequences, and promoters useful for expression of the desired nucleic acid sequence. In some of any embodiments, a plasmid comprises a promoter suitable for expression in a cell.

In some embodiments, the polynucleotides contain at least one promoter that is operatively linked to control expression of the targeted retargeted attachment protein and/or G protein and/or F protein. For expression of the retargeted attachment protein, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

In some embodiments, additional promoter elements, e.g., enhancers, regulate the frequency of transcriptional initiation. In some embodiments, additional promoter elements are located in the region 30-110 bp upstream of the start site, although a number of promoters have recently been shown to contain functional elements downstream of the start site as well. In some embodiments, spacing between promoter elements frequently is flexible, so that promoter function is preserved when elements are inverted or moved relative to one another. In some embodiments, the thymidine kinase (tk) promoter, the spacing between promoter elements can be increased to 50 bp apart before activity begins to decline. In some embodiments, depending on the promoter, individual elements can function either cooperatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202 and 5,928,906).

In some embodiments, a suitable promoter is the immediate early cytomegalovirus (CMV) promoter sequence. In some embodiments, the promoter sequence is a strong constitutive promoter sequence capable of driving high levels of expression of any polynucleotide sequence operatively linked thereto. In some embodiments, a suitable promoter is Elongation Growth Factor-la (EF-1 a). In some embodiments, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

In some embodiments, the promoter is an inducible promoter. In some embodiments, the inducible promoter provides a molecular switch capable of turning on expression of the polynucleotide sequence which it is operatively linked when such expression is desired, or turning off the expression when expression is not desired. In some embodiments, inducible promoters comprise metallothionine promoter, a glucocorticoid promoter, a progesterone promoter, and a tetracycline promoter.

In some embodiments, exogenously controlled inducible promoters can be used to regulate expression of the retargeted attachment protein, the G protein, the F protein, and/or an antigen binding domain such as a single domain antibody (sdAb) variable domain. For example, radiation-inducible promoters, heat-inducible promoters, and/or drug-inducible promoters can be used to selectively drive transgene expression in, for example, targeted regions. In such embodiments, the location, duration, and level of transgene expression can be regulated by the administration of the exogenous source of induction.

In some embodiments, expression of the retargeted attachment protein is regulated using a drug-inducible promoter. For example, in some cases, the promoter, enhancer, or transactivator comprises a Lac operator sequence, a tetracycline operator sequence, a galactose operator sequence, a doxycycline operator sequence, a rapamycin operator sequence, a tamoxifen operator sequence, or a hormone-responsive operator sequence, or an analog thereof. In some instances, the inducible promoter comprises a tetracycline response element (TRE). In some embodiments, the inducible promoter comprises an estrogen response element (ERE), which can activate gene expression in the presence of tamoxifen. In some instances, a drug-inducible element, such as a TRE, can be combined with a selected promoter to enhance transcription in the presence of drug, such as doxycycline. In some embodiments, the drug-inducible promoter is a small molecule-inducible promoter.

Any of the provided polynucleotides can be modified to remove CpG motifs and/or to optimize codons for translation in a particular species, such as human, canine, feline, equine, ovine, bovine, etc. species. In some embodiments, the polynucleotides are optimized for human codon usage (i.e., human codon-optimized). In some embodiments, the polynucleotides are modified to remove CpG motifs. In other embodiments, the provided polynucleotides are modified to remove CpG motifs and are codon-optimized, such as human codon-optimized. Methods of codon optimization and CpG motif detection and modification are well-known. Typically, polynucleotide optimization enhances transgene expression, increases transgene stability and preserves the amino acid sequence of the encoded polypeptide.

In order to assess the expression of the targeted envelope protein, the expression vector to be introduced into a cell can also contain either a selectable marker gene or a reporter gene or both to facilitate identification and selection of expressing particles, e.g. viral particles.

In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000, FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of the desired polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, one or more fusogens comprise at least one fusogen that has a tropism for cells permissible to lentiviral transduction, e.g., B cells, T cells, natural killer cells, islet cells, glial progenitor cells, neuronal cells, hematopoietic stem cells, cardiac cells, hepatocytes, stem cells, or induced pluripotent stem cells.

### III. Transgenes

The present disclosure provides viral vectors and methods for producing viral vectors that can be used for numerous purposes. For example, as discussed above, in some embodiments, viral vectors as described herein can include a transgene. In some embodiments, a transfer plasmid, which includes one or more transgenes, is used in the production of a viral vector. Acceptable transgenes that can be included in such viral vectors can widely vary and can be used for a large number of purposes. The only meaningful characteristic of such a transgene is that it be a nucleic acid that can be packaged and delivered by a viral vector.

In some embodiments, a transgene encodes a gene product. A gene product can be an RNA or a polypeptide.

In some embodiments, a transgene can encode an RNA. For example, a transgene can encode a gRNA, an siRNA, an shRNA, or miRNA.

In some embodiments, a transgene encodes a polypeptide.

In some embodiments, a transgene encodes a nuclease. In some embodiments, a nuclease is a Cas, a TALEN, or a zinc-finger nuclease. Viral vectors including transgenes encoding a nuclease can be useful for applications in which a viral vector is used to introduce genetic modifications into a cell. In such cases, a viral vector can enter into a cell and express the nuclease. In some embodiments, a viral vector may encode an RNA (e.g., a gRNA) and a polypeptide (e.g., a Cas) to impart targeted genetic modifications.

In some embodiments, a transgene encodes an antibody or portion thereof. Due to size limitations, in some embodiments, a transgene may encode an antibody having an alternative format that is smaller than a full canonical antibody (e.g., a Fab, a diabody, an scFV, a minibody, or nanobody). Viral vectors including transgenes encoding an antibody or portion thereof may be useful in applications involving targeted inhibition of molecules, e.g., molecules associated with specific cell types.

In some embodiments, a transgene encodes an antigen. Viral vectors including such transgenes can be helpful, e.g., in inducing desired immune responses.

In some embodiments, a transgene can encode a therapeutic polypeptide. In some embodiments, a transgene can encode a polypeptide used in protein replacement therapy. In some embodiments, a viral vector can include more than one transgene used in protein replacement therapy. For example, in some embodiments, a viral vector may include a first transgene encoding a nuclease that introduces a genetic modification knocking-out expression of an endogenous (e.g., dysfunctional) polypeptide and a second that delivers a (e.g., functional) replacement protein.

In some embodiments, a transgene can encode a chimeric antigen receptor (CAR). In some instances, a transgene encoding a CAR can be delivered to, e.g., a T-cell, for expression.

In some embodiments, the CAR binds to CD19. In some embodiments, the CAR binds to CD22. In some embodiments, the CAR binds to CD20. In some embodiments, the CAR binds to BCMA. In some embodiments, the CAR binds to an EBV antigen. In some embodiments, the CAR binds to CD27. In some embodiments, the CAR binds to CD30. In some embodiments, the CAR binds to CD19 and CD20. In some embodiments, the CAR binds to CD19 and CD22. In some embodiments, the CAR binds to CD19 and CD27. In some embodiments, the CAR binds to EBNA1. In some embodiments, the CAR binds to EBNA3A. In some embodiments, the CAR binds to BRLF1. In some embodiments, the CAR binds to BALF4. In some embodiments, the CAR binds to EBNA3C. In some embodiments, the CAR binds to LMP1. In some embodiments, the CAR binds to LMP2. In some embodiments, the CAR binds to LMP2A. In some embodiments, the CAR binds to LMP2B. In some embodiments, the CAR binds to BZLF1. In some embodiments, the CAR binds to BMLF1. In some embodiments, the CAR binds to gp350. In some embodiments, the CAR binds to gH/gL. In some embodiments, the CAR binds to EBNA1 and LMP1. In some embodiments, the CAR binds to EBNA1 and LMP2A. In some embodiments, the CAR binds to EBNA1, LMP1 and LMP2A. In some embodiments, the CAR binds to LMP, BARF1 and EBNA1. In some embodiments, the CAR binds to CD19 and an EBV antigen. In some embodiments, the CAR binds to CD20 and an EBV antigen. In some embodiments, the CAR binds to CD22 and an EBV antigen. In some embodiments, the CAR is selected from the group consisting of a first generation CAR, a second generation CAR, a third generation CAR, and a fourth generation CAR. In some embodiments, the CAR includes a single binding domain that binds to a single target antigen. In some embodiments, the CAR includes a single binding domain that binds to more than one target antigen, e.g., 2, 3, or more target antigens. In some embodiments, the CAR includes two binding domains such that each binding domain binds to a different target antigens. In some embodiments, the CAR includes two binding domains such that each binding domain binds to the same target antigen. Detailed descriptions of exemplary CARs including CD19-specific, CD20-specific and CD19/CD20-bispecific CARs can be found in WO2012/079000, WO2016/149578 and WO2020/014482, the disclosures including the sequence listings and figures are incorporated herein by reference in their entirety. In some embodiments, the CAR includes two binding domains such that each binding domain binds to the same target antigen. Detailed descriptions of exemplary CARs including CD19-specific, CD22-specific and CD19/CD22-bispecific CARs can be found in WO2012/079000, WO2016/149578 and WO2020/014482, the disclosures including the sequence listings and figures are incorporated herein by reference in their entirety. Detailed descriptions of exemplary CARs, TCRs or scFvs including CD27-specific, CD30-specific, EBNA1-specific, EBNA3C-specific, LMP1-specific, LMP2-specific, LMP2A-specific, gp350-specific CARs, gH/gL-specific CARs can be found in EP 2 520 589, U.S. Pat. Nos. 9,403,914, 11,180,566, US 2021/0009706, EP 2 558 498 B1, WO 2021/222929, US 2021/10206863, WO 2015/199617A1, WO 2012/109659A1, U.S. Pat. No. 7,786,269B2, WO 2021/211455A1, US 2016/0199479, WO2019/201995A1, and U.S. Pat. No. 11,116,835, the disclosures including the sequence listings and figures are incorporated herein by reference in their entireties. In some embodiments, the CAR includes two binding domains such that each binding domain binds to the same target antigen.

In some embodiments, the CD19 specific CAR includes an anti-CD19 single-chain antibody fragment (scFv), a transmembrane domain such as one derived from human CD8a, a 4-1BB (CD137) co-stimulatory signaling domain, and a CD35 signaling domain. In some embodiments, the CD20 specific CAR includes an anti-CD20 scFv, a transmembrane domain such as one derived from human CD8a, a 4-1BB (CD137) co-stimulatory signaling domain, and a CD35 signaling domain. In some embodiments, the CD19/CD20-bispecific CAR includes an anti-CD19 scFv, an anti-CD20 scFv, a transmembrane domain such as one derived from human CD8a, a 4-1BB (CD137) co-stimulatory signaling domain, and a CD32 signaling domain. In some embodiments, the CD22 specific CAR includes an anti-CD22 scFv, a transmembrane domain such as one derived from human CD8a, a 4-1BB (CD137) co-stimulatory signaling domain, and a CD35 signaling domain. In some embodiments, the CD19/CD22-bispecific CAR includes an anti-CD19 scFv, an anti-CD22 scFv, a transmembrane domain such as one derived from human CD8a, a 4-1BB (CD137) co-stimulatory signaling domain, and a CD32 signaling domain. In some embodiments, the EBNA1 specific CAR includes an anti-EBNA1 scFv, a transmembrane domain such as one derived from human CD8a, a 4-1BB (CD137) co-stimulatory signaling domain, and a CD32 signaling domain. In some embodiments, the EBNA3A CAR includes an anti-EBNA3A scFv, a transmembrane domain such as one derived from human CD8a, a 4-1BB (CD137) co-stimulatory signaling domain, and a CD35 signaling domain. In some embodiments, the EBNA3C CAR includes an anti-EBNA3C scFv, a transmembrane domain such as one derived from human CD8a, a 4-1BB (CD137) co-stimulatory signaling domain, and a CD32 signaling domain. In some embodiments, the LMP1 specific CAR includes an anti-LMP1 scFv, a transmembrane domain such as one derived from human CD8a, a 4-1BB (CD137) co-stimulatory signaling domain, and a CD3 signaling domain. In some embodiments, the LMP2 specific CAR includes an anti-LMP2 scFv, a transmembrane domain such as one derived from human CD8a, a 4-1BB (CD137) co-stimulatory signaling domain, and a CD32 signaling domain. In some embodiments, the LMP2A CAR includes an anti-LMP2A scFv, a transmembrane domain such as one derived from human CD8a, a 4-1BB (CD137) co-stimulatory signaling domain, and a CD32 signaling domain. In some embodiments, the BZLF1 CAR includes an anti-BZLF1 scFv, a transmembrane domain such as one derived from human CD8a, a 4-1BB (CD137) co-stimulatory signaling domain, and a CD35 signaling domain. In some embodiments, the BMLF1 CAR includes an anti-BMLF1 scFv, a transmembrane domain such as one derived from human CD8a, a 4-1BB (CD137) co-stimulatory signaling domain, and a CD35 signaling domain. In some embodiments, the gp350 CAR includes an anti-gp350 scFv, a transmembrane domain such as one derived from human CD8a, a 4-1BB (CD137) co-stimulatory signaling domain, and a CD32 signaling domain. In some embodiments, the gH/gL specific CAR includes an anti-gH/gL scFv, a transmembrane domain such as one derived from human CD8a, a 4-1BB (CD137) co-stimulatory signaling domain, and a CD3 signaling domain.

In some embodiments, the CAR comprises a commercial CAR construct carried by a T cell. Non-limiting examples of commercial CAR-T cell based therapies include brexucabtagene autoleucel (TECARTUS®), axicabtagene ciloleucel (YESCARTA®), idecabtagene vicleucel (ABECMA®), lisocabtagene maraleucel (BREYANZI®), tisagenlecleucel (KYMRIAH®), Descartes-08 and Descartes-11 from Cartesian Therapeutics, CTL119 from Novartis, P-BMCA-101 from Poseida Therapeutics, PBCAR19B and PBCAR269A from Precision Biosciences, FT819 from Fate Therapeutics, and CYAD-211 from Clyad Oncology.

In some embodiments, a hypoimmunogenic cell described herein comprises a polynucleotide encoding a chimeric antigen receptor (CAR) comprising an antigen binding domain. In some embodiments, a hypoimmunogenic cell described herein comprises a chimeric antigen receptor (CAR) comprising an antigen binding domain. In some embodiments, the polynucleotide is or comprises a chimeric antigen receptor (CAR) comprising an antigen binding domain. In some embodiments, the CAR is or comprises a first generation CAR comprising an antigen binding domain, a transmembrane domain, and at least one signaling domain (e.g., one, two or three signaling domains). In some embodiments, the CAR comprises a second generation CAR comprising an antigen binding domain, a transmembrane domain, and at least two signaling domains. In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an antibody, an antibody fragment, an scFv or a Fab.

In certain embodiments, the CAR may comprise a hinge domain, also referred to as a spacer. The terms “hinge” and “spacer” may be used interchangeably in the present disclosure. Non-limiting examples of hinge domains include CD8a hinge domain, CD28 hinge domain, IgG4 hinge domain, IgG4 hinge-CH2-CH3 domain, and variants thereof, the amino acid sequences of which are provided in Table 3 below.

TABLE 3
Exemplary sequences of hinge domains

| SEQ ID NO: | Sequence | Description |
|---|---|---|
| 26 | TTTPAPRPPTPAPTIASQPLSLRPEACRPAAGGAVHTRGL | CD8a hinge domain |
| | DFACD | |
| 27 | IEVMYPPPYLDNEKSNGTIIHVKGKHLCPSPLFPGPSKP | CD28 hinge domain |
| 28 | AAAIEVMYPPPYLDNEKSNGTIIHVKGKHLCPSPLFPGPS | CD28 hinge domain |
| | KP | |
| 29 | ESKYGPPCPPCP | IgG4 hinge domain |
| 30 | ESKYGPPCPSCP | IgG4 hinge domain |
| 31 | ESKYGPPCPPCPAPEFLGGPSVFLFPPKPKDTLMISRTPE | IgG4 hinge-CH2-CH3 |
| | VTCVVVDVSQEDPEVQFNWYVDGVEVHNAKTKPREEQF | domain |
| | NSTYRVVSVLTVLHQDWLNGKEYKCKVSNKGLPSSIEKTI | |
| | SKAKGQPREPQVYTLPPSQEEMTKNQVSLTCLVKGFYPS | |
| | DIAVEWESNGQPENNYKTTPPVLDSDGSFFLYSRLTVDK | |
| | SRWQEGNVFSCSVMHEALHNHYTQKSLSLSLGK | |

In certain embodiments, the transmembrane domain of the CAR may comprise a transmembrane region of the alpha, beta, or zeta chain of a T cell receptor, CD28, CD38, CD45, CD4, CD5, CD8, CD9, CD16, CD22, CD33, CD37, CD64, CD80, CD86, CD134, CD137, CD154, or a functional variant thereof, including the human versions of each of these sequences. In other embodiments, the transmembrane domain may comprise a transmembrane region of CD8a, CD8B, 4-1BB/CD137, CD28, CD34, CD4, FcaRly, CD16, OX40/CD134, CD3ζ, CD38, CD3γ, CD38, TCRa, TCRB, TCR, CD32, CD64, CD64, CD45, CD5, CD9, CD22, CD37, CD80, CD86, CD40, CD40L/CD154, VEGFR2, FAS, and FGFR2B, or a functional variant thereof, including the human versions of each of these sequences. Table 4 provides the amino acid sequences of a few exemplary transmembrane domains.

TABLE 4
Exemplary sequences of transmembrane domains

| SEQ ID NO: | Sequence | Description |
|---|---|---|
| 32 | IYIWAPLAGTCG | CD8α transmembrane |
| | VLLLSLVITLYC | domain |
| 33 | FWVLVVVGGVLA | CD28 transmembrane |
| | CYSLLVTVAFII | domain |
| | FWV | |
| 34 | MFWVLVVVGGVL | CD28 transmembrane |
| | ACYSLLVTVAFI | domain |
| | IFWV | |

In certain embodiments, the intracellular signaling domain and/or intracellular costimulatory domain of the CAR may comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFB, OX40/TNFRSF4, OX40 Ligand/TNFSF4, RELT/TNFRSF19L, TACI/TNFRSF13B, TL1A/TNFSF15, TNFa, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thyl, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD35, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain and/or intracellular costimulatory domain comprises one or more signaling domains selected from a CD35 domain, an ITAM, a CD28 domain, 4-1BB domain, or a functional variant thereof. Table 5 provides the amino acid sequences of a few exemplary intracellular costimulatory and/or signaling domains. In certain embodiments, the CD35 signaling domain of SEQ ID NO:37 may have a mutation, e.g., a glutamine (Q) to lysine (K) mutation, at amino acid position 14 (see SEQ ID NO:38).

TABLE 5
Exemplary sequences of intracellular costimulatory and/or signaling domains

| SEQ ID NO: | Sequence | Description |
|---|---|---|
| 35 | KRGRKKLLYIFKQPFMRPVQTTQEEDGCSCRFPEE | 4-1BB costimulatory domain |
| | EEGGCEL | |
| 36 | RSKRSRLLHSDYMNMTPRRPGPTRKHYQPYAPPR | CD28 costimulatory domain |
| | DFAAYRS | |
| 37 | RVKFSRSADAPAYQQGQNQLYNELNLGRREEYDV | CD3ζ signaling domain |
| | LDKRRGRDPEMGGKPRRKNPQEGLYNELQKDKM | |
| | AEAYSEIGMKGERRRGKGHDGLYQGLSTATKDTY | |
| | DALHMQALPPR | |
| 38 | RVKFSRSADAPAYKQGQNQLYNELNLGRREEYDV | CD3ζ signaling domain (with |
| | LDKRRGRDPEMGGKPRRKNPQEGLYNELQKDKM | Q to K mutation at position |
| | AEAYSEIGMKGERRRGKGHDGLYQGLSTATKDTY | 14) |
| | DALHMQALPPR | |

In some embodiments, a CD19 specific CAR is comprises an FMC63 anti-CD19 variable domain (FMC63 CAR). In some embodiments, a CD19 specific CAR (e.g., a CAR19) comprises an FMC63 anti-CD19 scFv. In some embodiments, a cell that carries an FMC63 CAR targets a cell (e.g., B cells) that expresses CD19.

In some embodiments, the extracellular binding domain of the CD19 CAR comprises an scFv derived from the FMC63 monoclonal antibody (FMC63), which comprises the heavy chain variable region (VH) and the light chain variable region (VL) of FMC63 connected by a linker. FMC63 and the derived scFv have been described in Nicholson et al., Mol. Immun. 34 (16-17): 1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 6 below. In some embodiments, the CD19-specific scFv comprises or consists of an amino acid sequence set forth in SEQ ID NO: 39, 40, or 45, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 39, 40, or 45. In some embodiments, the CD19-specific scFv may comprise one or more CDRs having amino acid sequences set forth in SEQ ID NOs: 41-43 and 46-48. In some embodiments, the CD19-specific scFv may comprise a light chain with one or more CDRs having amino acid sequences set forth in SEQ ID NOs: 41-43. In some embodiments, the CD19-specific scFv may comprise a heavy chain with one or more CDRs having amino acid sequences set forth in SEQ ID NOs: 46-48. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the VII and the VL portions of the scFv is a Whitlow linker having an amino acid sequence set forth in SEQ ID NO:44. In some embodiments, the Whitlow linker may be replaced by a different linker, for example, a 3×G4S linker having an amino acid sequence set forth in SEQ ID NO:50, which gives rise to a different FMC63-derived scFv having an amino acid sequence set forth in SEQ ID NO:49. In certain of these embodiments, the CD19-specific scFv comprises or consists of an amino acid sequence set forth in SEQ ID NO:49 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:49.

TABLE 6
Exemplary sequences of anti-CD19 scFv and components

| SEQ ID NO: | Amino Acid Sequence | Description |
|---|---|---|
| 39 | DIQMTQTTSSLSASLGDRVTISCRASQDI | Anti-CD19 FMC63 scFv |
| | SKYLNWYQQKPDGTVKLLIYHTSRLHSG | entire sequence, with |
| | VPSRFSGSGSGTDYSLTISNLEQEDIATYF | Whitlow linker |
| | CQQGNTLPYTFGGGTKLEITGSTSGSGKP | |
| | GSGEGSTKGEVKLQESGPGLVAPSQSLSV | |
| | TCTVSGVSLPDYGVSWIRQPPRKGLEWL | |
| | GVIWGSETTYYNSALKSRLTIIKDNSKSQ | |
| | VFLKMNSLQTDDTAIYYCAKHYYYGGSY | |
| | AMDYWGQGTSVTVSS | |
| 40 | DIQMTQTTSSLSASLGDRVTISCRASQDI | Anti-CD19 FMC63 scFv |
| | SKYLNWYQQKPDGTVKLLIYHTSRLHSG | light chain variable |
| | VPSRFSGSGSGTDYSLTISNLEQEDIATYF | region |
| | CQQGNTLPYTFGGGTKLEIT | |
| 41 | QDISKY | Anti-CD19 FMC63 scFv |
| | | light chain CDR1 |
| 42 | HTS | Anti-CD19 FMC63 scFv |
| | | light chain CDR2 |
| 43 | QQGNTLPYT | Anti-CD19 FMC63 scFv |
| | | light chain CDR3 |
| 44 | GSTSGSGKPGSGEGSTKG | Whitlow linker |
| 45 | EVKLQESGPGLVAPSQSLSVTCTVSGVSL | Anti-CD19 FMC63 scFv |
| | PDYGVSWIRQPPRKGLEWLGVIWGSET | heavy chain variable |
| | TYYNSALKSRLTIIKDNSKSQVFLKMNSL | region |
| | QTDDTAIYYCAKHYYYGGSYAMDYWGQ | |
| | GTSVTVSS | |
| 46 | GVSLPDYG | Anti-CD19 FMC63 scFv |
| | | heavy chain CDR1 |
| 47 | IWGSETT | Anti-CD19 FMC63 scFv |
| | | heavy chain CDR2 |
| 48 | AKHYYYGGSYAMDY | Anti-CD19 FMC63 scFv |
| | | heavy chain CDR3 |
| 49 | DIQMTQTTSSLSASLGDRVTISCRASQDI | Anti-CD19 FMC63 scFv |
| | SKYLNWYQQKPDGTVKLLIYHTSRLHSG | entire sequence, with |
| | VPSRFSGSGSGTDYSLTISNLEQEDIATYF | 3xG₄S linker |
| | CQQGNTLPYTFGGGTKLEITGGGGSGGG | |
| | GSGGGGSEVKLQESGPGLVAPSQSLSVTC | |
| | TVSGVSLPDYGVSWIRQPPRKGLEWLGV | |
| | IWGSETTYYNSALKSRLTIIKDNSKSQVF | |
| | LKMNSLQTDDTAIYYCAKHYYYGGSYAM | |
| | DYWGQGTSVTVSS | |
| 50 | GGGGSGGGGSGGGGS | 3xG₄S linker |

In some embodiments, the extracellular binding domain of the CD19 CAR is derived from an antibody specific to CD19, including, for example, SJ25C1 (Bejcek et al., Cancer Res. 55:2346-2351 (1995)), HD37 (Pezutto et al., J. Immunol. 138 (9): 2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB 12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148 (10): 2983-2987 (1992)), and CLB-CD19 (De Rie Cell. Immunol. 118:368-381 (1989)). In any of these embodiments, the extracellular binding domain of the CD19 CAR can comprise or consist of the VH, the VL, and/or one or more CDRs of any of the antibodies.

In some embodiments, the hinge domain of the CD19 CAR comprises a CD8a hinge domain, for example, a human CD8a hinge domain. In some embodiments, the CD8a hinge domain comprises or consists of an amino acid sequence set forth in SEQ ID NO:26 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:26. In some embodiments, the hinge domain comprises a CD28 hinge domain, for example, a human CD28 hinge domain. In some embodiments, the CD28 hinge domain comprises or consists of an amino acid sequence set forth in SEQ ID NO:27 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:27. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:29 or SEQ ID NO:30, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:29 or SEQ ID NO:30. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:31 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:31.

In some embodiments, the transmembrane domain of the CD19 CAR comprises a CD8a transmembrane domain, for example, a human CD8a transmembrane domain. In some embodiments, the CD8a transmembrane domain comprises or consists of an amino acid sequence set forth in SEQ ID NO:32 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:32. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 33 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:33.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO:35 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:35. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO:36 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:36. In some embodiments, the intracellular costimulatory domain of the CD19 CAR comprises a 4-1BB costimulatory domain and a CD28 costimulatory domain as described.

In some embodiments, the intracellular signaling domain of the CD19 CAR comprises a CD3 zeta (2) signaling domain. CD32 associates with T cell receptors (TCRs) to produce a signal and contains immunoreceptor tyrosine-based activation motifs (ITAMs). The CD32 signaling domain refers to amino acid residues from the cytoplasmic domain of the zeta chain that are sufficient to functionally transmit an initial signal necessary for T cell activation. In some embodiments, the CD32 signaling domain is human. In some embodiments, the CD32 signaling domain comprises or consists of an amino acid sequence set forth in SEQ ID NO:37 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:37.

In some embodiments, a transgene can encode a chimeric B-cell autoantibody receptor (BAR). A BAR recognizes and binds to certain antibody-expressing B cells. In some embodiments, a BAR comprises an antigen. An antigen of a BAR can be bound by neutralizing antibodies. The neutralizing antibodies may be undesirable because they can block or inhibit an effect or function of antigen to which they bind. For example, hemophilia patients can receive therapeutic factor VIII (FVIII) as part of their treatment. However, a patient's body may develop an immune response against the FVIII, including the production of anti-FVIII antibodies from B cells. When the patient produces anti-FVIII antibodies that bind to FVIII, FVIII is not able to perform its therapeutic functions. Accordingly, it may be beneficial to remove the anti-FVIII antibodies and/or the B-cells producing those antibodies from the patient. A BAR, which includes an FVIII antigen, can be used for this purpose.

In some embodiments, a BAR comprises a transmembrane domain. In some embodiments, a BAR comprises a signaling domain. In some embodiments, a BAR comprises one or more signaling domains.

In some embodiments, a BAR comprises an antigen, a transmembrane domain, and a signaling domain. In some embodiments, a BAR comprises an antigen, a transmembrane domain, and one or more signaling domains.

A BAR can be expressed by, e.g., a hypoimmunogenic T-cell. BAR T-cells can recognize and can bind target select antibodies and/or the B cells producing those antibodies. Once a BAR T-cell binds a target antibody, the BAR T-cell can destroy the antibodies and/or the B cells producing those antibodies. In some embodiments, a BAR T-cell is a BAR T-cell (Treg), e.g., a regulatory T-cell (Treg) comprising a BAR.

A BAR can be expressed by, e.g., a hypoimmunogenic NK-cell. BAR NK-cells can recognize and can bind target select antibodies and/or the B cells producing those antibodies. Once a BAR NK-cell binds a target antibody, the BAR NK-cell can destroy the antibodies and/or the B cells producing those antibodies.

In some embodiments, a transgene can encode a chimeric autoantibody receptor (CAAR). In some embodiments, a CAAR comprises an antigen, e.g., an autoantigen that can be bound by autoantibodies. In some embodiments, a CAAR comprises a transmembrane domain. In some embodiments, a CAAR comprises a signaling domain. In some embodiments, a CAAR comprises one or more signaling domains. In some embodiments, a CAAR comprises an antigen, a transmembrane domain, and a signaling domain. In some embodiments, a CAAR comprises an antigen, a transmembrane domain, and one or more signaling domains.

A CAAR can be expressed by, e.g., a hypoimmunogenic T-cell. CAAR T-cells can recognize and can bind target autoantibodies expressed on autoreactive cells via an antigen of a CAAR. Once a CAAR T-cell binds a target autoantibody expressed on an autoreactive cell, the CAAR T-cell can destroy the autoreactive cell.

A CAAR can be expressed by, e.g., a hypoimmunogenic NK-cell. CAAR NK-cells can recognize and can bind target autoantibodies expressed on autoreactive cells via an antigen of a CAAR. Once a CAAR NK-cell binds a target autoantibody expressed on an autoreactive cell, the CAAR NK-cell can destroy the autoreactive cell.

In some embodiments, a transgene can encode a tolerogenic factor. In some embodiments, a tolerogenic factor is CD47, DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, CD16, CD52, H2-M3, CD16 Fc receptor, IL15-RF, and Serpinb9, A20/TNFAIP3, CD39, CR1, HLA-F, IL15-RF, or MANF. In some embodiments, a tolerogenic factor is CD47.

In some embodiments, a viral vector includes one or more transgenes. In some embodiments, a viral vector includes one, two, three, four, five or more transgenes.

In some embodiments, one or more nucleic acids for the production of the viral vector can include one or more transgenes. In some embodiments, one or more nucleic acids for the production of the viral vector include one, two, three, four, five or more transgenes.

In some embodiments, one or more nucleic acids for the production of the viral vector include a transfer plasmid. In some embodiments, a transfer plasmid can include one or more transgenes. In some embodiments, a transfer plasmid includes one, two, three, four, five or more transgenes.

In some embodiments, a transgene can encode a chimeric antigen receptor (CAR). In some instances, a transgene encoding a CAR can be delivered to, e.g., a T-cell, for expression.

### IV. Methods for Producing Lentivirus Vectors

Disclosed herein are methods of manufacturing a drug product that includes producing viral vectors, e.g., lentiviral vectors and detecting replication competent lentivirus (RCL) before, during, or after producing the viral vectors.

Typically, a producer cell line is transfected with one or more plasmids (e.g., as disclosed herein), and cultured under conditions sufficient to transfer and/or express one or more polypeptides encoded by said plasmids in the producer cell line.

Methods for producing a composition comprising a viral vector include introducing into a producer cell (e.g., transfecting a producer cells) one or more nucleic acids (e.g., plasmids) described herein. Nucleic acids (e.g., plasmids) that can be used for transfecting a produce cell include: one or more packaging plasmids, one or more envelope plasmids, one or more regulatory plasmids, and one or more transfer plasmids as described herein.

In some embodiments, a ratio of an envelope plasmid to a packaging plasmid is about 1:1.

In some embodiments, a relative amount of an envelope plasmid is increased, e.g., to increase the amount of an encoded fusogen present in a produced lentiviral vector. In some embodiments, the fusogen is an attachment protein. In some embodiments, a fusogen is a paramyxovirus G protein or a portion thereof. In some embodiments, a fusogen is a chimeric protein that comprises a paramyxovirus G protein or a biologically active portion thereof and an scFV. A fusogen can also comprise other fusogens as described herein.

In some embodiments, a ratio of an envelope plasmid to a packaging plasmid is greater than 1:1, greater than 2:1, greater than 3:1, greater than 4:1, greater than 5:1. In some embodiments, a ratio of an envelope plasmid to a packaging plasmid is greater than 1:1. In some embodiments, a ratio of an envelope plasmid to a packaging plasmid is at least 2:1. In some embodiments, a ratio of an envelope plasmid to a packaging plasmid is a ratio of said plasmids within a producer cell.

In some embodiments, a ratio of an envelope plasmid to a packaging plasmid is about 1.5:1, about 2:1, about 4:1, about 5:1, about 6:1, about 7:1, about 8:1, about 9:1, about 10:1, about 11:1, about 12:1, about 13:1, about 14:1, or about 15:1. In some embodiments, a ratio of an envelope plasmid to a packaging plasmid is about 1.5:1. In some embodiments, a ratio of an envelope plasmid to a packaging plasmid is about 2:1. In some embodiments, a ratio of an envelope plasmid to a packaging plasmid is a ratio of said plasmids within a producer cell.

In some embodiments, a ratio of an envelope plasmid to a packaging plasmid is between 1:1 to 2:1, between 1:1 to 4:1, between 1:1 to 5:1, between 1.5:1 to 6:1, between 1.5:1 to 7:1, between 1.5:1 to 8:1, between 1.5:1 to 9:1, between 1.5:1 to 10:1, between 1.5:1 to 11:1, between 1.5:1 to 12:1, between 1.5:1 to 13:1, between 1.5:1 to 14:1, or between 1.5:1 to 15:1. In some embodiments, a ratio of an envelope plasmid to a packaging plasmid is a ratio of said plasmids within a producer cell.

In some embodiments, a ratio of an envelope plasmid to a packaging plasmid is at most 15:1, at most 14:1, at most 13:1, at most 12:1, at most 11:1, at most 10:1, at most 9:1, or at most 8:1. In some embodiments, a ratio of an envelope plasmid to a packaging plasmid is a ratio of said plasmids within a producer cell.

In some embodiments, a ratio of an envelope plasmid to a transfer plasmid is at least 1:1, at least 2:1, at least 3:1, at least 4:1, or at least 5:1. In some embodiments, a ratio of an envelope plasmid to a transfer plasmid is at least 1:1. In some embodiments, a ratio of an envelope plasmid to a transfer plasmid is at least 2:1. In some embodiments, a ratio of an envelope plasmid to a transfer plasmid is a ratio of said plasmids within a producer cell.

In some embodiments, a ratio of an envelope plasmid to a transfer plasmid is about 1:1, about 2:1, about 4:1, about 5:1, about 6:1, about 7:1, about 8:1, about 9:1, about 10:1, about 11:1, about 12:1, about 13:1, about 14:1, or about 15:1. In some embodiments, a ratio of an envelope plasmid to a transfer plasmid is about 1:1. In some embodiments, a ratio of an envelope plasmid to a transfer plasmid is about 2:1. In some embodiments, a ratio of an envelope plasmid to a transfer plasmid is a ratio of said plasmids within a producer cell.

In some embodiments, a ratio of an envelope plasmid to a transfer plasmid is between 1:1 to 2:1, between 1:1 to 4:1, between 1:1 to 5:1, between 1:1 to 6:1, between 1:1 to 7:1, between 1:1 to 8:1, between 1:1 to 9:1, between 1:1 to 10:1, between 1:1 to 11:1, between 1:1 to 12:1, between 1:1 to 13:1, between 1:1 to 14:1, or between 1:1 to 15:1. In some embodiments, a ratio of an envelope plasmid to a transfer plasmid is a ratio of said plasmids within a producer cell.

In some embodiments, a ratio of an envelope plasmid to a transfer plasmid is at most 15:1, at most 14:1, at most 13:1, at most 12:1, at most 11:1, at most 10:1, at most 9:1, or at most 8:1. In some embodiments, a ratio of an envelope plasmid to a transfer plasmid is a ratio of said plasmids within a producer cell.

In some embodiments, two envelope plasmids are introduced to a producer cell. In some embodiments, a first and a second envelope plasmid each encoding a different fusogen (e.g., a first fusogen and a second fusogen, respectively) are introduced to a producer cell. In some embodiments, a ratio of a first envelope plasmid to a second envelope plasmid is at least 1:1, at least 2:1, at least 4:1, at least 6:1, at least 8:1, at least 10:1, at least 12:1, or at least 15:1. In some embodiments, a ratio of an envelope plasmid to a packaging plasmid is greater than 1:1. In some embodiments, a ratio of a first envelope plasmid to a second envelope plasmid is a ratio of said plasmids within a producer cell. In some embodiments, a first envelope plasmid encodes G protein and a second envelope plasmid encodes F protein.

In some embodiments, a ratio of a first envelope plasmid to a second envelope plasmid is between 1:1 to 2:1, between 1:1 to 4:1, between 1:1 to 6:1, between 1:1 to 8:1, between 1:1 to 10:1, between 1:1 to 12:1, or between 1:1 to 15:1. In some embodiments, a ratio of a first envelope plasmid to a second envelope plasmid is a ratio of said plasmids within a producer cell. In some embodiments, a first envelope plasmid encodes G protein and a second envelope plasmid encodes F protein.

In some embodiments, a ratio of a first envelope plasmid to a second envelope plasmid is at most 24:1, at most 22:1, at most 20:1, at most 18:1, or at most 16:1. In some embodiments, a ratio of a first envelope plasmid to a second envelope plasmid is a ratio of said plasmids within a producer cell. In some embodiments, a first envelope plasmid encodes G protein and a second envelope plasmid encodes F protein.

In some embodiments, a ratio of a first envelope plasmid to a second envelope plasmid is 1:1, is 2:1, is 4:1, is 6:1, is 8:1, is 10:1, or is 12:1. In some embodiments, a first envelope plasmid encodes G protein and a second envelope plasmid encodes F protein.

In some embodiments, a ratio of first envelope plasmid to second envelope plasmid is optimized for increased expression of G protein on a lentiviral vector. In some embodiments the lentiviral vector is a fusosome. In some embodiments, a ratio of first envelope plasmid to second envelope plasmid is optimized to increase expression of G protein by a producer cell. In some embodiments, a ratio of first envelope plasmid to second envelope plasmid is optimized so a greater proportion of fusosomes comprise both G protein and F protein (e.g., complete fusosome). In some embodiments, a ratio of one fusogen relative to another fusogen on a lentiviral vector (e.g., fusosome) as described herein, confers tropism of the lentiviral vector to one or more cell(s) (e.g., B cells, T cells, natural killer cells, islet cells, glial progenitor cells, neuronal cells, hematopoietic stem cells, cardiac cells, hepatocytes, stem cells, or induced pluripotent stem cells). In some embodiments, a lentiviral vector (e.g., fusosome) with tropism to one more or cell(s) carries one or more CAR(s). In some embodiments, an optimized ratio of G protein relative to F protein produces a lentiviral vector (e.g., fusosome) that targets CD8 positive T cells. [add] In some embodiments, a lentiviral vector (e.g., fusosome) that targets CD8 positive T cells carries a nucleic acid sequence encoding a FMC63 CAR.

In some embodiments, a lentiviral vector encodes a FMC63 CAR (e.g., fusosome), which targets CD8 positive T cells. CD8 positive T cells can express a FMC63 CAR, which allows the CD8 positive T cells to target CD19 expressing cells (e.g., B cell) via an affinity of the FMC63 CAR with CD19.

In some embodiments, introduction of nucleic acids into producer cells occurs by transfection. In some embodiments, transfection can be performed using lipofectamine. In some embodiments, a 2:1 ratio of lipofectamine to DNA is used for transfection. In some embodiments, transfection can be performed with electroporation. In some embodiments, transfection can be performed with nucleofection.

In some embodiments, one or more of the one or more nucleic acids for the production of the viral vectors are stably integrated (e.g., stable integration results in stable expression of the encoded viral proteins) into the genome of one or more producer cells. In some embodiments, one or more of the one or more nucleic acids for the production of the viral vectors are stably integrated (e.g., stable integration results in stable expression of the encoded viral proteins) into the genome of one or more producer cells prior to culturing the producer cells under conditions sufficient to produce the viral vectors. In some embodiments, stable integration is achieved via random (i.e., insertion into a random genomic locus of the host cell) integration of the one or more nucleic acids into the genome of a producer cell. In some embodiments, stable integration is achieved via targeted integration (i.e., insertion into a specific genomic locus of the host cell) into the genome of a producer cell. As known to a person skilled in the art, viral vectors, including, for example, retroviral vectors, lentiviral vectors, adenoviral vectors, and adeno-associated viral vectors, are commonly used to deliver genetic material into producer cells and randomly insert the gene(s) encoding viral proteins into the host cell genome to facilitate stable expression and replication of the viral genes.

A number of gene editing methods can be used to insert a polynucleotide (e.g., gene encoding viral proteins for production of a viral vector) into a specific genomic locus of choice. Gene editing is a type of genetic engineering in which a nucleotide sequence is inserted, deleted, modified, or replaced in the genome of a living organism. In some embodiments, the gene editing technologies are systems involving nucleases, integrases, transposases, and/or recombinases. In some embodiments, the gene editing technology mediates single-strand breaks (SSB). In some embodiments, the gene editing technology mediates double-strand breaks (DSB), including in connection with non-homologous end-joining (NHEJ) or homology-directed repair (HDR). In some embodiments, the gene editing technologies are DNA-based editing or prime-editing. In some embodiments, the gene editing technology is Programmable Addition via Site-specific Targeting Elements (PASTE). In some embodiments, the gene editing technology is TnpB polypeptides. Many gene editing techniques generally utilize the innate mechanism for cells to repair double-strand breaks (DSBs) in DNA.

Eukaryotic cells repair DSBs by two primary repair pathways: non-homologous end-joining (NHEJ) and homology-directed repair (HDR). HDR typically occurs during late S phase or G2 phase, when a sister chromatid is available to serve as a repair template. NHEJ is more common and can occur during any phase of the cell cycle, but it is more error prone. In gene editing, NHEJ is generally used to produce insertion/deletion mutations (indels), which can produce targeted loss of function in a target gene by shifting the open reading frame (ORF) and producing alterations in the coding region or an associated regulatory region. HDR, on the other hand, is a preferred pathway for producing targeted knock-ins, knockouts, or insertions of specific mutations in the presence of a repair template with homologous sequences. Several methods are known to a skilled artisan to improve HDR efficiency, including, for example, chemical modulation (e.g., treating cells with inhibitors of key enzymes in the NHEJ pathway); timed delivery of the gene editing system at S and G2 phases of the cell cycle; cell cycle arrest at S and G2 phases; and introduction of repair templates with homology sequences. The methods provided herein may utilize HDR-mediated repair, NHEJ-mediated repair, or a combination thereof.

In some embodiments, the methods provided herein for HDR-mediated insertion utilize a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems.

### A. Cell Culture

The present disclosure, in some embodiments, provides methods for introducing one or more nucleic acids into cells permissible to lentiviral transduction for the production of viral vectors. In some embodiments, cells permissible to lentiviral transduction are capable of producing a viral vector when cultured under appropriate conditions. In some embodiments, cells permissible to lentiviral transduction are cultured at 37° C. In some embodiments, cells permissible to lentiviral transduction are cultured at least at 37° C. In some embodiments, cells permissible to lentiviral transduction are cultured between 35° C. and 45° C. In some embodiments, cells permissible to lentiviral transduction are cultured while exposed to, among other gases, 5% carbon dioxide. In some embodiments, cells permissible to lentiviral transduction are cultured at a temperature that is permissive to viral vector production. In some embodiments, cells permissible to lentiviral transduction are cultured with one or more gas(es) that is/are permissive to viral vector production. In some embodiments, cells permissible to lentiviral transduction are cultured in a 96 well plate, a T25 flask, a T75 flask, a T150 flask, or a T225 flask.

In some cases, “cell culture” and “producer cell culture” are used interchangeably.

In some embodiments, cells permissible to lentiviral transduction are any one of a number of cells known to be capable of producing viral vectors. In some embodiments, cells permissible to lentiviral transduction comprise one or more B cells, T cells, natural killer cells, islet cells, glial progenitor cells, neuronal cells, hematopoietic stem cells, cardiac cells, hepatocytes, stem cells, induced pluripotent stem cells, or SupT1 cells In some embodiments, cells permissible to lentiviral transduction comprise T cells. In some embodiments, cells permissible to lentiviral transduction comprise SupT1 cells. In some embodiments, cells permissible to lentiviral transduction are transfected with one or more lentiviral vector plasmids. In some embodiments, cells permissible to lentiviral transduction are transduced with a lentiviral vector. In some embodiments, cells permissible to lentiviral transduction are transduced with murine leukemia virus (MLV).

In some embodiments, a producer cell is any one of a number of cells known to be capable of producing viral vectors. In some embodiments, a producer cell comprises one or more HEK293 cells, PER.C6 cells, VERO cells, HEK 293T cells, A549 cells, MRC5 cells, HeLa cells, Sf9 cells, and BHK-21 cells.

In some embodiments, a producer cell (e.g., HEK 293 or HEK 293T cells) is adapted to serum-free growth. In some embodiments, a producer cell is adapted to suspension culture. In some embodiments, adapted (e.g., to serum-free media and/or suspension culture) producer cell clones are selected based on production of a higher crude functional titer of lentiviral vector. In some embodiments, adapted producer cell clones are selected based on morphology. In some embodiments, adapted producer cell clones are selected based on higher productivity of a fusosome. In some embodiments, adapted producer cell clones are selected based on higher productivity of a fusosome targeting one or more cell(s). In some embodiments, adapted producer cell clones are selected based on higher productivity of a CD8-targeted fusosome. In some embodiments, adapted producer cell clones are selected based on fusosomes that carry a CAR transgene. In some embodiments, adapted producer cell clones are selected based on fusosomes that carry a CD19-directed CAR transgene. In some embodiments, adapted producer cell clones are selected based on one or more of the following; increase in crude functional lentiviral vector titer, morphology, increase in productivity of a specific fusosomes, increase in productivity of CD8-targeting fusosomes, increase in productivity of fusosomes that carry a CAR transgene, increase in productivity of fusosomes that carry a CD19-directed CAR transgene. In some embodiments, adapted producer cell clones are selected based on feature(s) (e.g., functional titer, morphology, productivity, transgene expression) exhibited relative to each other, to a control cell line that has not been adapted to serum-free growth in a suspension culture, or both.

In some embodiments, cells permissible to lentiviral transduction are cultured in a suspension culture. In some embodiments, a cell culture comprises a suspension culture. In some embodiments, a cell culture comprises a culture of cells permissible to lentiviral transduction. In some embodiments, a cell culture is transduced with a vector, e.g., lentiviral vector, MLV. In some embodiments, the test sample as described herein is obtained from a cell culture. In some embodiments, the test sample as described herein comprises supernatant from a cell culture.

The method of the present disclosure comprises amplification of a nucleic acid derived from a control source, e.g., MLV, and amplification of a nucleic acid derived from a source separate from a control, e.g., lentiviral vector. In some embodiments, both sources (e.g., MLV and lentiviral vector) are transduced into cells permissible to lentiviral transduction. In some embodiments, cells transduced with a lentiviral vector are in a different, e.g., separate, independent, culture as cells transduced with MLV. In some embodiments, cells transduced with a lentiviral vector are in the same culture as cells transduced with MLV.

In some embodiments, the method of manufacturing a drug product includes producing viral vectors, e.g., lentiviral vectors, where producing includes contacting the viral vector (e.g., as a drug substance) with a nuclease, and detecting replication competent lentivirus (RCL) before, during, or after producing the viral vectors. For example, the method of producing a composition comprising viral vectors, includes (a) culturing producer cells comprising one or more nucleic acids for the production of the viral vector under conditions sufficient to produce the viral vectors, wherein the one or more nucleic acids comprise a packaging plasmid, an envelope plasmid, and a transfer plasmid; (b) treating the producer cell culture with a nuclease; and (c) harvesting the producer cell culture; wherein (b) is performed prior (c), wherein the method include detecting replication competent lentivirus, before or after treating the producer cell culture with a nuclease.

### B. Fusogen

Disclosed herein are methods of producing viral vectors, e.g., lentiviral vectors, comprising one or more fusogens. Also disclosed are one or more nucleic acids for the production of viral vectors (e.g., lentiviral vectors) that comprise one or more nucleic acid sequences encoding a fusogen. In some embodiments, one or more fusogen encoded by a nucleic acid is a viral fusogen. In some embodiments, one or more fusogens comprise at least one fusogen that is involved in attachment of a viral vector to a cell membrane. In some embodiments, one or more fusogens comprise at least one fusogen that is involved in directing fusion of the lipid bilayer of a viral vector to a cell membrane. In some embodiments, one or more fusogens comprise one or more paramyxovirus envelope proteins or portion thereof. In some embodiments, one or more paramyxovirus envelope proteins or portion thereof comprise a paramyxovirus glycoprotein (“Protein G”) or portion thereof. In some embodiments, one or more paramyxovirus envelope proteins or a portion thereof comprise a paramyxovirus fusion protein (“Protein F”) or a portion thereof.

In some embodiments, one or more fusogens comprise at least one fusogen that has a tropism for cells permissible to lentiviral transduction, e.g., B cells, T cells, natural killer cells, islet cells, glial progenitor cells, neuronal cells, hematopoietic stem cells, cardiac cells, hepatocytes, stem cells, or induced pluripotent stem cells.

### V. Nucleic Acid Amplification

In some embodiments, methods of the present disclosure use techniques for amplification of nucleic acid. In some embodiments, nucleic acid amplification techniques include but are not limited to, PCR, real-time PCR, quantitative PCR, and droplet digital PCR (ddPCR). One of skill in the art will recognize that various nucleic acid amplification techniques may be useful in the context of the present disclosure, see, e.g., as described in Sambrook et al., Molecular Cloning: A Laboratory Manual (2d ed., Cold Spring Harbor Laboratory Press, Cold Spring Harbor, N.Y. (1989)), which is incorporated herein by reference for any purpose). In some embodiments, a nucleic acid amplification technique is droplet digital PCR (ddPCR).

A. Methods for Performing ddPCR

The present disclosure provides, among other things, methods for performing ddPCR (e.g., to detect RCL). ddPCR can offer multiple advantages over regular PCR and/or real-time PCR, including but not limited to, data are absolute so that standard curves are not needed, data are more comparable across time points and physical locations of experiments, and no replicate wells are needed due to high precision measurements.

In some embodiments, ddPCR comprises combining multiple components into a reaction mixture. In some embodiments, a reaction mixture comprises a test sample comprising test nucleic acids. In some embodiments, a reaction mixture comprises primers, probes, and polymerase enzymes. In some embodiments, a reaction mixture comprises a manufacturer's solution comprising, e.g., primers, probes, and polymerase enzymes. In some embodiments, a reaction mixture comprises oil.

In some embodiments, ddPCR utilizes a water-oil emulsion droplet system. In some embodiments, a reaction mixture is emulsified so that droplets are formed that partition and separate test nucleic acid molecules. In some embodiments, the process of emulsification to form droplets involves microfluidics technology. In some embodiments, thousands of droplets are formed within a single reaction mixture as a result of emulsification. In some embodiments, droplets are roughly the same volume (~1 nL). In some embodiments, PCR occurs in each individual droplet in an independent reaction. In some embodiments, if a target nucleic acid is present within a droplet, amplification occurs and increased fluorescence results within the droplet. In some embodiments, if a target nucleic acid is not present within a droplet, amplification does not occur and weak, residual fluorescence results within the droplet. In some embodiments, amplification occurs from thermal cycling, comparable to amplification protocols for standard PCR.

In some embodiments, droplet fluorescence is measured after a set number of thermal cycles to achieve amplification. In some embodiments, droplets that contain a certain threshold of fluorescence are determined to be positive for target nucleic acid. In some embodiments, droplets that do not contain a certain threshold of fluorescence are determined to be negative for target nucleic acid. In some embodiments, droplet fluorescence is measured by an automated droplet reader. In some embodiments, droplet fluorescence is measured by streaming all droplets in a single file through a fluorescence detector.

In some embodiments, the ratio of positive droplets to negative droplets determines the concentration of target nucleic acid in the test sample. In some embodiments, target nucleic acid amplicon amount is normalized using a normalization amplicon. In some embodiments, a normalization amplicon is encoded by a genomic reference sequence. In some embodiments, a normalization amplicon is encoded by a genomic reference sequence for human and non-human primate genomes, for example, uTert: GACGACGTGCTGGTTCACCTGCTGGCACGCTGCGCGCTCTTTGTGCTGGTGGCTCCC AGCTGCGCCTACCAGGTGTGCGGGCC (SEQ ID NO: 1). In some embodiments, a normalization amplicon is encoded by a genomic reference sequence for human genome only, for example, hTert:

(SEQ ID NO: 2)
GGCACACGTGGCTTTTCGCTCAGGACGTCGAGTGGACACGGTGATCTC
TGCCTCTGCTCTCCCTCCTGTCCAGTTTGCATAAACTTACGAGGTTCA
CC.

In some embodiments, ddPCR is performed using the Bio-Rad QX100 Droplet Digital PCR system.

B. Methods for Detecting RCL with ddPCR

In some embodiments, ddPCR is performed to detect RCL. In some embodiments, ddPCR is performed to detect RCL comprising a fusogen. In some embodiments, ddPCR is performed to amplify a nucleic acid encoding at least a portion of a fusogen.

In some embodiments, a fusogen for amplification as described herein comprises one or more paramyxovirus envelope proteins or portion thereof. In some embodiments, a fusogen has a tropism for cells permissible to lentiviral transduction. In some embodiments, one or more paramyxovirus envelope proteins or portiona thereof comprises a paramyxovirus glycoprotein (“Protein G”) or a portion thereof. In some embodiments, one or more paramyxovirus envelope proteins or portion thereof comprises a paramyxovirus fusion protein (“Protein F”) or a portion thereof.

In some embodiments, ddPCR is performed to amplify a nucleic acid encoding a control sequence from a control virus. In some embodiments, the control virus is capable of transducing a cell line or tissue type. In some embodiments, the control virus is capable of transducing one or more of B cells, T cells, natural killer cells, islet cells, glial progenitor cells, neuronal cells, hematopoietic stem cells, cardiac cells, hepatocytes, stem cells, induced pluripotent stem cells, or SupT1 cells. In some embodiments, the control virus is capable of transducing SupT1 cells or a derivative thereof. In some embodiments, the control virus is HIV-1. In some embodiments, the control virus is MLV (e.g., 4070A-MLV). In some embodiments, the control sequence from the control virus encodes MLV Env protein or a portion thereof. In some embodiments, a control amplicon is detected to confirm cell permissibility to lentiviral infection and/or to confirm ddPCR has been performed.

In some embodiments, ddPCR is performed on a test sample obtained from a cell culture comprising cells permissible to lentiviral transduction. In some embodiments, the test sample comprises supernatant from a cell culture comprising cells permissible to lentiviral transduction. In some embodiments, the cell culture comprising cells permissible to lentiviral transduction have been transduced with a lentiviral vector. In some embodiments, the cells have been transduced with MLV. In some embodiments, the cells have been transduced with MLV at a concentration of, for example, 1 IU, 10 IU, or 100 IU. In some embodiments, the cells have been transduced with pseudotyped lentiviral vector (PLV).

### VI. Drug Substance

In some embodiments, methods provided herein are useful for the production of viral vectors. In some embodiments, such viral vectors are utilized in a drug substance. A “drug substance” is an active ingredient (e.g., viral vectors) that is intended to furnish pharmacological activity or other direct effect in the diagnosis, cure, mitigation, treatment, or prevention of disease or to affect the structure or any function of a subject's body, but does not include intermediates used in the synthesis of such ingredient. A drug substance may need further processing to become a “drug product,” which is a finished dosage form (e.g., tablet or solution) to be administered to a subject. However, a drug substance does not require further processing to purify, isolate, or otherwise enrich the active ingredient prior to incorporation into a drug product.

In some embodiments, a method comprises generating a drug substance from a cell culture, where the drug substance comprises viral vectors produced by cells permissible to lentiviral transduction or a subset thereof. In some embodiments, a cell culture is transduced with a drug substance comprising a lentiviral vector. In some embodiments, a test sample as described herein is obtained from a cell culture that has been transduced with a drug substance comprising a lentiviral vector.

In some embodiments, a drug substance or drug product as described herein can include a pharmaceutically acceptable carrier or excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference) discloses various excipients used in formulating pharmaceutical compositions and known techniques for the preparation thereof. Suitable pharmaceutically acceptable carriers include but are not limited to water, salt solutions (e.g., NaCl), saline, buffered saline, glycerol, sugars such as mannitol, sucrose, or others, dextrose, fatty acid esters, etc., as well as combinations thereof.

In one embodiment, a method of manufacturing a drug product, comprises detecting replication competent lentivirus (RCL) in the drug product by: (i) performing a nucleic acid amplification technique on a reaction mixture, wherein the reaction mixture comprises a test sample that comprises test nucleic acid, wherein the nucleic acid amplification technique is capable of amplifying a nucleic acid encoding at least a portion of the fusogen, wherein a fusogen amplicon will be produced if the nucleic acid encoding the fusogen of the RCL is present in the test nucleic acid; and (ii) determining whether the fusogen amplicon is produced by the nucleic acid amplification technique, wherein the presence of the fusogen amplicon indicates presence of RCL.

In one embodiment, a method of manufacturing a drug product, comprises detecting replication competent lentivirus (RCL) in the drug product by: (i) performing a nucleic acid amplification technique on a reaction mixture, wherein the reaction mixture comprises a test sample that comprises test nucleic acid, wherein the nucleic acid amplification technique is capable of amplifying a nucleic acid encoding at least a portion of the fusogen, wherein a fusogen amplicon will be produced if the nucleic acid encoding the fusogen of the RCL is present in the test nucleic acid; and (ii) determining whether the fusogen amplicon is produced by the nucleic acid amplification technique, wherein the presence of the fusogen amplicon indicates presence of RCL, wherein absence of the fusogen amplicon satisfies a release criteria for the drug product.

### VII. Kits

Described herein are kits for us in detecting replication competent lentivirus (RCL). In some embodiments, the kits are used in a method of manufacturing a drug product, where the kit is used to detected replication competent lentivirus (RCL) in a drug product.

The disclosure is further illustrated by the following examples. The examples are provided for illustrative purposes only. They are not to be construed as limiting the scope or content of the disclosure in any way.

### EXAMPLES

The following examples are provided so as to describe to the skilled artisan how to make and use methods and compositions described herein, and are not intended to limit the scope of the present disclosure.

### Example 1: Validation of Replication Competent Lentivirus (RCL) Assay

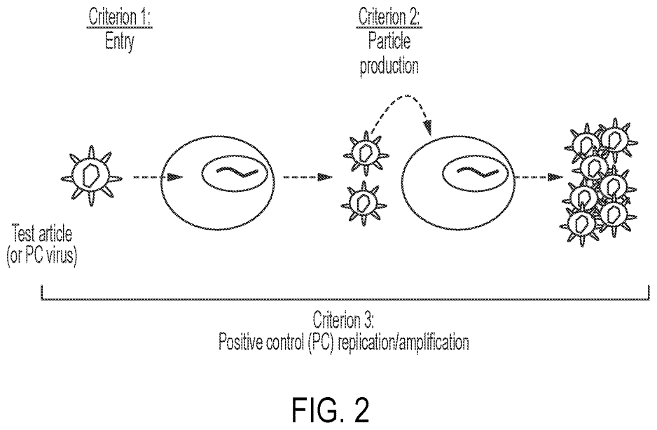

The present Example describes an assay to assess RCL formation. Various criteria for lentiviral production are shown in FIG. 2, with RCL assay(s) relating specifically to criterion 3. An exemplary pseudotyped lentiviral vector (PLV) was utilized in experiments as described herein (e.g., a lentiviral vector pseudotyped with a retargeted paramyxovirus envelope targeting CD8 for in vivo gene delivery).

On Day 0, SupT1 cells were plated into 10 different T25 flasks in 12.5 mLs of RPMI+10% FBS at 4×10⁶ total cells. Three flasks were also simultaneously inoculated with PLV, three positive control flasks were simultaneously inoculated with 1IU, 10IU, and 100IU of MLV, one flask was simultaneously inoculated with 5×10³:1 PLV:MLV, one flask was simultaneously inoculated with 5×10⁴:10 PLV:MLV, and one flask remained untransduced. On Day 1, flasks were topped up with media to 25 mLs. On Days 3, 6, and 9, cells were passaged 1:4 into T25 flasks after being washed with media twice, and a subset of cells was harvested for ddPCR analysis. In some instances, ddPCR analysis can also be performed after Day 9.

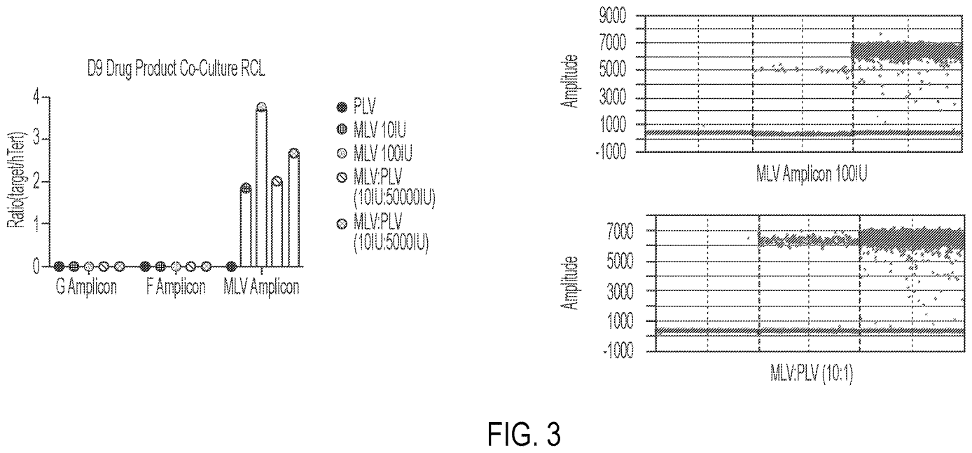

ddPCR analysis was performed for G Amplicon, F Amplicon, and MLV Amplicon. Amplicon signals were normalized to SupT1 normalization amplicon (e.g., hTert) and exemplary ddPCR amplitude cycles are shown in FIG. 3. MLV outgrowth was quantifiable (e.g., signal was above the lower limit of quantification (LLOQ)) in all control conditions after passage 3. No signal was detected for G and F Amplicons (e.g., signal was below the lower limit of detection (LLOD)) in all conditions. This data indicated replication competent PLV was not produced in SupT1 cells. Interference controls displayed a decreased outgrowth of MLV but still were above LLOQ.

### Example 2: Assessment of Amplification and Indicator Phases of Novel RCL Assay in Larger-Scale Co-Culture Experiments

The present Example describes assessment of RCL formation and MLV outgrowth across different co-culture conditions at a larger scale. NivF and NivG depletion was assessed in culture at different time points.

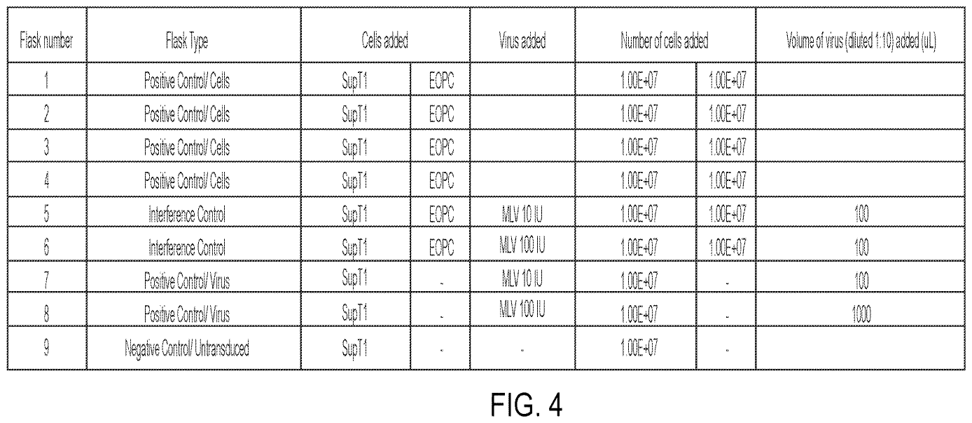

An exemplary experimental setup is shown in FIG. 4. On Day 0, end of production cells (EOPC; 1×10⁷ cells) and SupT1 cells (1×10⁷ cells) were co-inoculated in a T225 flask. On Day 1, 1IU, 10IU, and 100IU of crude MLV was spiked into certain co-culture flasks. On Day 2, flasks were topped up with media to 40 mLs. On Days 3 and 6, cells were passaged at a ratio of 1:10, and a subset of cells was harvested for ddPCR analysis. On Day 9, co-culture supernatant was harvested, and cells were harvested for ddPCR analysis. Data from these time points are part of the amplification phase, during which any RCL present would replicate. To begin the indicator phase, 25 mL of supernatant from Day 9 of the amplification phase was added to fresh SupT1 cells (1×10⁷ cells). On Day 10, flasks were topped up with media to 40 mLs. On Days 12 and 15, cells were passaged 1:10, and a subset of cells was harvested for ddPCR analysis. On Day 18, cells were harvested and genomic DNA (gDNA) was extracted for ddPCR analysis on G, F, and MLV Amplicons. Data from these time points are part of the indicator phase, during which any RCL that might have grown out during the amplification phase would be detected.

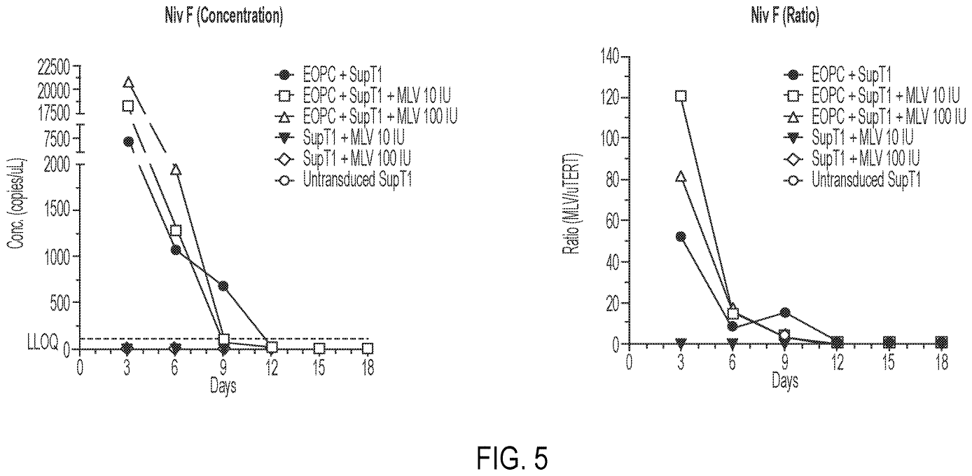

ddPCR signal for F Amplicon (here, NivF) in copies per μL and normalized to SupT1 normalization amplicon (e.g., uTert) is shown in FIG. 5. The F Amplicon was observed to deplete over the course of the amplification phase (e.g., Day 3 to Day 9). As predicted, no amplification was observed during the indicator phase (e.g., Day 12 to Day 18). These data indicate that replication competent PLV was not produced in SupT1 cells from these co-culture experiments at larger scale. Interference controls (samples comprising PLV and MLV) were below LLOD by Day 9, and leveled out to 0 signal at Day 12. A similar trend was observed between copies per μL and when normalized to uTert.

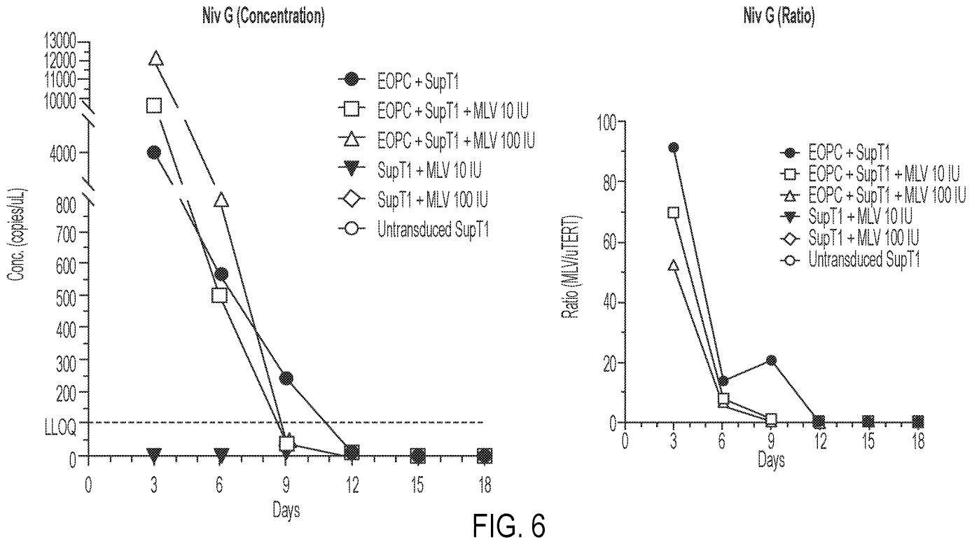

ddPCR signal for G Amplicon (here, NivG) in copies per μL and normalized to SupT1 normalization amplicon (e.g., uTert) is shown in FIG. 6. Similar trends were observed for these data relative to F Amplicon data.

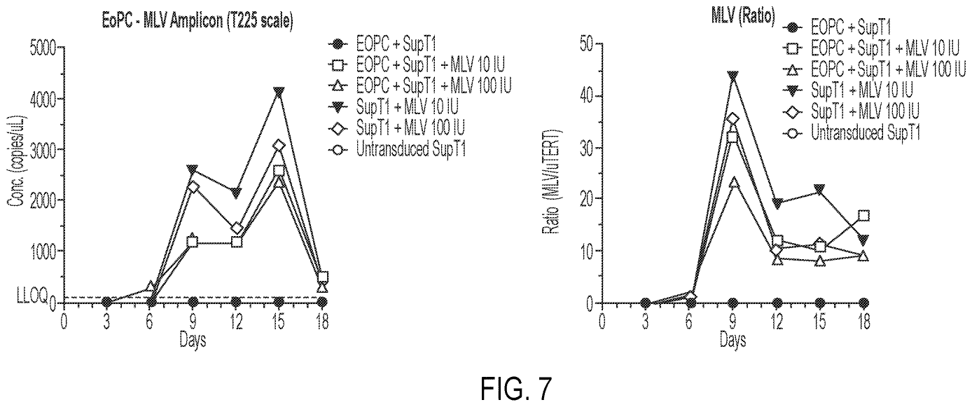

ddPCR signal for MLV Amplicon in copies per μL and normalized to SupT1 normalization amplicon (e.g., uTert) is shown in FIG. 7. MLV outgrowth was observed across all IU concentrations. Increased MLV outgrowth was observed in EOPC co-culture conditions relative to mono-culture conditions. Slightly different trends were observed between copies per μL and when normalized to uTert. As expected, no MLV amplification was observed in conditions where MLV was not inoculated.

These data demonstrate that consistent replication of MLV was observed, establishing that SupT1 cells are permissive of MLV amplification. This effect was observed across different MLV concentrations (e.g., 1IU, 10IU, and 1000IU) and across different experimental scales (e.g., 96 well plates, T25 and T225 flasks). Both NivF and NivG fell below the LLOQ around Day 9, and leveled out close to 0 signal over the course of the assay.

Traditional RCL assays utilize the C8166 cell line and HIV-1 as a positive control with readouts based on p24 protein, qPCR, or PERT to assess for outgrowth of replication competent particles. Due to the low sensitivity associated with these assays, these RCL assays can take 50-86 days to reach endpoint readout and they require significant amounts of vector material to complete. Furthermore, use of HIV-1 as a positive control introduces biosafety challenges. While C8166 cells are permissive to VSV-G pseuodotyped LV commonly used in ex vivo cell therapy, these cells may not be permissive to the latest vectors being developed for in vivo use with selective tropism.

These data demonstrate the development and validation of a novel RCL assay that is ddPCR based, utilizes SupT1 cells, and incorporates 4070A-MLV as a positive control. Importantly, this RCL assay decreases assay time from 50-86 days down to 9-18 days and allows for sensitive quantification of positive control outgrowth while decreasing the amount of vector test material needed.

### EQUIVALENTS

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of detecting replication competent lentivirus (RCL) comprising a fusogen, the method comprising:
(a) performing a nucleic acid amplification technique on a reaction mixture,
wherein the reaction mixture comprises a test sample that comprises test nucleic acid,
wherein the nucleic acid amplification technique is capable of amplifying a nucleic acid encoding at least a portion of the fusogen,
wherein a fusogen amplicon will be produced if the nucleic acid encoding the fusogen of the RCL is present in the test nucleic acid; and
(b) determining whether the fusogen amplicon is produced by the nucleic acid amplification technique, wherein the presence of the fusogen amplicon indicates presence of RCL.

2. The method of claim 1, wherein the nucleic acid technique is capable of amplifying a nucleic acid encoding a control sequence from a control virus, wherein a control amplicon will be produced if the nucleic acid encoding the control sequence is present in the test nucleic acid.

3. The method of claim 2, comprising determining whether the control amplicon is produced by the nucleic acid amplification technique, wherein the presence of the control amplicon indicates the nucleic acid amplification technique was successfully performed.

4. The method of claim 2 or 3, wherein the control virus is mouse leukemia virus (MLV) and the control sequence is a nucleic acid sequence encoding at least a portion of an MLV Env protein.

5. The method of claim 1, wherein the nucleic acid amplification technique is single molecule PCR.

6. The method of claim 5, wherein the nucleic acid amplification technique is ddPCR.

7. The method of claim 1, wherein the test sample is obtained from a cell culture comprising cells permissible to lentiviral transduction.

8. The method of claim 7, wherein the method further comprises obtaining the test sample from the cell culture.

9. The method of claim 8, wherein the test sample comprises supernatant from the cell culture.

10. The method of claim 7, wherein the cells permissible to lentiviral transduction have been transduced with a lentiviral vector.

11. The method of claim 7 or 10, wherein the cells permissible to lentiviral transduction have been transduced with MLV.

12. The method of claim 11, wherein the cells transduced with a lentiviral vector and the cells transduced with MLV are in different cultures.

13. The method of claim 11, wherein the cells transduced with a lentiviral vector and the cells transduced with MLV are in the same cultures.

14. The method of any one of claims 7-13, wherein the transduction occurs in a 96 well plate, a T25 flask, a T75 flask, a T150 flask, or a T225 flask.

15. The method of any one of claims 7-14, wherein the cells in the culture are passaged at least once between transduction and collection of the supernatant.

16. The method of any one of claims 7-15, wherein the cells in the culture are passaged at least twice between transduction and collection of the supernatant.

17. The method of any one of claims 7-16, wherein the cells transduced with MLV are transduced at 1 infectious unit (IU), 10 IU, or 100 IU.

18. The method of claim 7, where the cells permissible to lentiviral transduction are selected from B cells, T cells, natural killer cells, islet cells, glial progenitor cells, neuronal cells, hematopoietic stem cells, cardiac cells, hepatocytes, stem cells, or induced pluripotent stem cells.

19. The method of claim 18, wherein the cells permissible to lentiviral transduction are T cells.

20. The method of claim 19, wherein the cells permissible to lentiviral transduction are SupT1 cells.

21. The method of any one of claims 7-20, wherein the test sample is obtained at least 7, 8, 9, or 10 days after transduction with the lentiviral vector.

22. The method of any one of claims 7-20 wherein the test sample is obtained less than 56 days after transduction with the lentiviral vector.

23. The method of any one of claims 1-22, wherein the fusogen encoded by the nucleic acid is a viral fusogen.

24. The method of any one of claims 1-23, wherein the fusogen is involved in attachment of a viral vector to a cell membrane.

25. The method of any one of claims 1-24, wherein the fusogen is involved in directing fusion of the lipid bilayer of a viral vector and a cell membrane.

26. The method of any one of claims 1-25, wherein the fusogen comprises one or more paramyxovirus envelope proteins or portion thereof.

27. The method of claim 26, wherein the one or more paramyxovirus envelope proteins or portion thereof comprises a paramyxovirus glycoprotein (“Protein G”) or a portion thereof.

28. The method of claim 26 or 27, wherein the one or more paramyxovirus envelope proteins or portion thereof comprises a paramyxovirus fusion protein (“Protein F”) or portion thereof.

29. The method of any one of claims 1-28, wherein the fusogen has a tropism for the cells permissible to lentiviral transduction.

30. A method of manufacturing a drug product, comprising:
performing the method of any one of claims 1-29, wherein the test sample was obtained from a cell culture that had been transduced with a drug substance comprising a lentiviral vector.

31. The method of claim 30, wherein the fusogen amplicon is not produced by the nucleic acid amplification technique.

32. The method of claim 30 or 31, wherein the control amplicon is produced by the nucleic acid amplification technique.

33. The method of any one of claims 30-32, further comprising adding a pharmaceutically acceptable excipient to the drug substance.

34. The method of any one of claims 30-32, wherein the test sample is collected prior to harvesting the cell culture.

35. The method of any one of claims 30-34, further comprising treating the cell culture with a nuclease.

36. The method of claim 35, wherein the test sample is collected before treating the cell culture with a nuclease.

37. The method of claim 35, wherein the test sample is collected after treating the cell culture with a nuclease.

38. The method of any one of claims 1-37, wherein the presence of the fusogen amplicon above a predetermined threshold indicates presence of RCL.

39. A method of manufacturing a drug product, comprising
detecting replication competent lentivirus (RCL) in the drug product by:
(i) performing a nucleic acid amplification technique on a reaction mixture,
wherein the reaction mixture comprises a test sample that comprises test nucleic acid,
wherein the nucleic acid amplification technique is capable of amplifying a nucleic acid encoding at least a portion of the fusogen,
wherein a fusogen amplicon will be produced if the nucleic acid encoding the fusogen of the RCL is present in the test nucleic acid; and
(ii) determining whether the fusogen amplicon is produced by the nucleic acid amplification technique, wherein the presence of the fusogen amplicon indicates presence of RCL.
